(19) United States
(12) Patent Application Publication
(10) Pub. No.: US 2005/0065187 A1
(43) Pub. Date: Mar. 24, 2005

(54) Novel antimicrobial aryloxazolidinone compounds

(76) Inventors: Mikhail Fedorovich Gordeev, Castro Valley, CA (US); Qiang Wang, Berkeley, CA (US)

Correspondence Address: WARNER-LAMBERT COMPANY, 2800 PLYMOUTH RD, ANN ARBOR, MI, 48105, US

(21) Appl. No.: 10/924,752

(22) Filed: Aug. 24, 2004

Related U.S. Application Data
(60) Provisional application No. 60/497,531, filed on Aug. 25, 2003.

(51) Int. Cl. A61K 31/454; A61K 31/421; C07D 413/02

(52) U.S. Cl. 514/326; 514/376; 514/378; 514/473; 546/209; 548/221; 548/243

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

### FIELD OF INVENTION

The present invention relates to novel aryloxazolidinone compounds containing a dihydropyridone subunit, and related methods of preparation. The invention compounds are active against Gram-positive and Gram-negative bacteria.

### BACKGROUND OF THE INVENTION

Oxazolidinones represent a novel synthetic class of antimicrobials with potent activity against a number of human and veterinary pathogens, including Gram-positive aerobic bacteria such as multiply-resistant staphylococci and streptococci, anaerobic organisms such as bacteroides and clostridia species, and acid-fast organisms such as Mycobacterium tuberculosis and Mycobacterium avium.

However, because oxazolidinones generally do not demonstrate activities at a useful level against aerobic Gram-negative organisms, their use is limited to infectious states due to Gram-positive bacteria. Accordingly, it is among the objects of the present invention to provide oxazolidinone compounds that have broader antibacterial activity, including the activity against aerobic Gram-negative organisms.

### SUMMARY OF THE INVENTION

These and other needs are met by the present invention which is directed to a compound of formula I 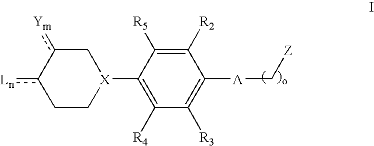
or a pharmaceutically acceptable salt thereof wherein:
“- - - ” is a bond or is absent;
A is a structure selected from the group consisting of i, ii, iii, and iv 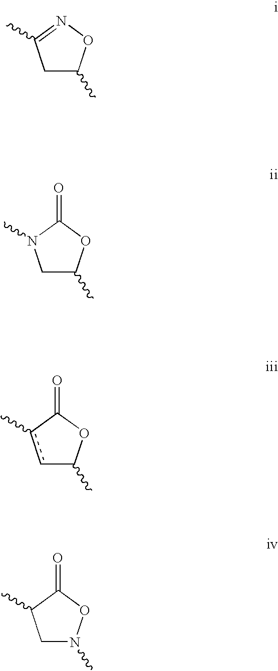
   wherein “- - - ” is a bond or is absent and “ [symbol] ” indicate points of attachment;
X is N, or C;
Z is
   (a) NHC(═O)R¹,
   (b) NHC(═S)R¹,
   (c) NH-het¹,
   (d) O-het¹,
   (e) S-het¹,
   (f) het²; or
   (g) CONHR¹, wherein
   R¹ is
   (a) H,
   (b) NH₂,
   (c) NHC₁₋₄alkyl,
   (d) C₁₋₄alkyl,
   (e) C₂₋₄alkenyl,
   (f) C₁₋₄heteroalkyl,
   (g) (CH₂)ₚC(═O)C₁₋₄alkyl,
   (h) OC₁₋₄alkyl, except when o=0;
   (i) SC₁₋₄alkyl, except when o=0;
   (j) (CH₂)ₚC₃₋₆cycloalkyl,
   (k) CH₂C(═O)-aryl, or
   (1) CH₂C(═O)-het;
R², R³, R⁴ and R are each independently
   (a) H,
   (b) Cl,
   (c) F,
   (d) CH₃,
   (e) NH₂, or
   (f) OH;
L and Y are each independently
   (a) H,
   (b) OH,
   (c) F,
   (d) O,
   (e) NOH,
   (f) NOR;
m, n, o, p are each independently 0 or 1.

The invention is also directed to a compound of formula II 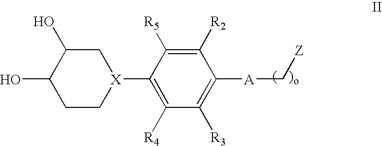
or a pharmaceutically acceptable salt thereof, wherein X, R₂, R₃, R⁴, R₅, and o have the definitions as provided for compounds of formula I.

The invention is also directed to a compound of formula III 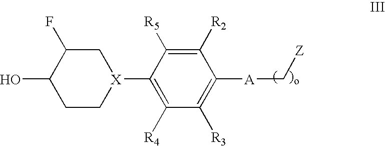
or a pharmaceutically acceptable salt thereof, wherein X, R₂, R₃, R⁴, R₅, and o have the definitions as provided for compounds of formula I.

The invention is also directed to a compound of formula IV 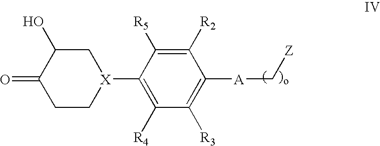
or a pharmaceutically acceptable salt thereof, wherein X, R₂, R₃, R⁴, R₅, and o have the definitions as provided for compounds of formula I.

The invention is also directed to a compound of formula V 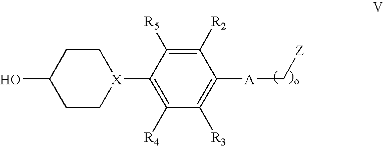
or a pharmaceutically acceptable salt thereof, wherein X, R₂, R₃, R⁴, R₅, and o have the definitions as provided for compounds of formula I.

The invention is also directed to a compound of formula VI 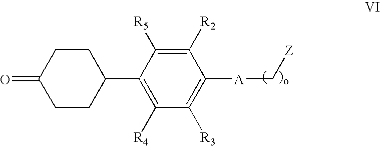
or a pharmaceutically acceptable salt thereof, wherein R₂, R₃, R⁴, R₅, and o have the definitions as provided for compounds of formula I.

The invention is also directed to a compound of formula VII 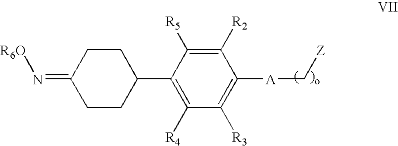
or a pharmaceutically acceptable salt thereof, wherein R₂, R₃, R⁴, R₅, and o have the definitions as provided for compounds of formula I, and R₆ is H or (C₁-C₆)alkyl.

The invention is also directed to a compound which is:
(a) N-{3-[4-(3,4-Dihydroxy-cyclohexyl)-phenyl]-2-oxo-oxazolidin-5-ylmethyl}-acetamide;
(b) 2,2-Dichloro-N-{3-[4-(3,4-dihydroxy-cyclohexyl)-phenyl]-2-oxo-oxazolidin-5-ylmethyl}-acetamide;
(c) N-{3-[4-(3,4-Dihydroxy-cyclohexyl)-phenyl]-2-oxo-oxazolidin-5-ylmethyl}-2,2-difluoro-acetamide;
(d) N-{3-[4-(3,4-Dihydroxy-cyclohexyl)-phenyl]-2-oxo-oxazolidin-5-ylmethyl}-2,2-difluoro-thioacetamide;
(e) N-{3-[4-(3,4-Dihydroxy-cyclohexyl)-3-fluoro-phenyl]-2-oxo-oxazolidin-5-ylmethyl}-acetamide;
(f) 2,2-Dichloro-N-{3-[4-(3,4-dihydroxy-cyclohexyl)-3-fluoro-phenyl]-2-oxo-oxazolidin-5-ylmethyl}-acetamide;
(g) N-{3-[4-(3,4-Dihydroxy-cyclohexyl)-3-fluoro-phenyl]-2-oxo-oxazolidin-5-yl methyl}-2,2-difluoro-acetamide;
(h) N-{3-[4-(3,4-Dihydroxy-cyclohexyl)-3-fluoro-phenyl]-2-oxo-oxazolidin-5-ylmethyl}-2,2-difluoro-thioacetamide;
(i) N-{3-[3-Fluoro-4-(4-hydroxy-cyclohexyl)-phenyl]-2-oxo-oxazolidin-5-ylmethyl}-acetamide;
(j) N-{3-[3-Fluoro-4-(4-oxo-cyclohexyl)-phenyl]-2-oxo-oxazolidin-5-ylmethyl}-acetamide;
(k) 2,2-Difluoro-N-{3-[3-fluoro-4-(4-hydroxy-cyclohexyl)-phenyl]-2-oxo-oxazolidin-5-ylmethyl}-thioacetamide;
(1) N-{3-[3-Fluoro-4-(4-hydroxyimino-cyclohexyl)-phenyl]-2-oxo-oxazolidin-5-ylmethyl}-acetamide;
(m) N-{3-[3-Fluoro-4-(4-hydroxy-cyclohexyl)-phenyl]-2-oxo-oxazolidin-5-ylmethyl}-propionamide;
(n) N-{3-[3-Fluoro-4-(3-hydroxy-4-oxo-cyclohexyl)-phenyl]-2-oxo-oxazolidin-5-ylmethyl}-acetamide;
(o) 1-[2-Fluoro-4-(2-oxo-5-[1,2,3]triazol-1-ylmethyl-oxazolidin-3-yl)-phenyl]-3-hydroxy-piperidin-4-one;
(p) 3-[4-(3,4-Dihydroxy-piperidin-1-yl)-3-fluoro-phenyl]-5-[1,2,3]triazol-1-ylmethyl-oxazolidin-2-one;
(q) 3-[3-Fluoro-4-(4-hydroxy-cyclohexyl)-phenyl]-2-oxo-oxazolidine-5-carboxylic acid amide;
(r) 3-[3-Fluoro-4-(4-oxo-cyclohexyl)-phenyl]-2-oxo-oxazolidine-5-carboxylic acid amide;
(s) N-{3-[3-Fluoro-4-(3-fluoro-4-hydroxy-cyclohexyl)-phenyl]-2-oxo-oxazolidin-5-ylmethyl}-acetamide; or
(t) 2,2-Difluoro-N-{3-[3-fluoro-4-(3-fluoro-4-hydroxy-cyclohexyl)-phenyl]-2-oxo-oxazolidin-5-ylmethyl}-thioacetamide.

The invention is further directed to a pharmaceutical composition comprising a compound of formula I, II, i, IV, V, VI, or VII or a pharmaceutically acceptable salt thereof, admixed with a pharmaceutically acceptable excipent, carrier, or diluent.

The invention is further directed to a method for treating a microbial infection in a mammal in need of such treatment, comprising administering a therapeutically effective amount of a compound of formula I, II, I, IV, V, VI, or VII or a pharmaceutically acceptable salt thereof.

The invention is further directed to a method for treating gram-positive microbial infections in a mammal in need of such treatment, comprising administering a therapeutically effective amount of a compound of formula I, II, III, IV, V, VI, or VII or a pharmaceutically acceptable salt thereof, and

The invention is further directed to a method for treating a gram-negative microbial infection in a mammal in need of such treatment, comprising administering a therapeutically effective amount of a compound of formula I, II, III, IV, V, VI, or VII or a pharmaceutically acceptable salt thereof.

### DETAILED DESCRIPTION OF THE INVENTION

The following definitions are used, unless otherwise described.

The term alkyl, alkenyl, etc. refer to both straight and branched groups, but reference to an individual radical such as “propyl” embraces only the straight chain radical, a branched chain isomer such as “isopropyl” being specifically referred to.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms, inclusive.

Alkyl, alkenyl, or cycloalkyl groups optionally may be substituted with one, two, or three substituents selected from the group consisting of halo, aryl, het¹, and het¹.

The term “halo” refers to fluoro (F), chloro (Cl), bromo (Br), or iodo (I).

Aryl is phenyl, biphenyl, or naphthyl, optionally substituted with halo, —C1-4alkyl, —OH, —OC₁₋₄alkyl, —S(═O)ₙC1-4alkyl, and —C₁₋₄alkyl-NH₂.

The term “het” is a C-linked five-(5) or six-(6) membered heterocyclic or heteroaryl ring having 1-4 heteroatoms selected from the group consisting of oxygen, sulfur, and nitrogen. Het¹ may be substituted where it is suitable; and may be an ortho-fused bicyclic heterocycle of about eight to ten ring atoms derived there from, particularly a benz-derivative or one derived by fusing a propylene, trimethylene, or tetramethylene diradical thereto.

Examples of “het¹” include, but are not limited to, pyridine, thiophene, furan, pyrazole, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,5-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl and 5-isothiazolyl, 1,3,4,-oxadiazole, 4-oxo-2-thiazolinyl, or 5-methyl-1,3,4-thiadiazol-2-yl, thiazoledione, 1,2,3,4-thiatriazole, or 1,2,4-dithiazolone.

The term “het²” is a N-linked five-(5) or six-6) membered heterocyclic or heteroaryl ring having at least one nitrogen atom, and optionally having one oxygen or sulfur atom. Het may be substituted where it is suitable.

Examples of “het²” include, but are not limited to, 1,2,3-triazolyl, 1,2,4-triazolyl, pyrrolyl, imidazolyl, pyrazolyl, triazolyl, tetrazolyl or isoxazolinonyl.

Mammal refers to human or animals including livestock and companion animals.

When a bond is represented by a symbol such as “- - - ” this is meant to represent that the bond may be absent or present provided that the resultant compound is stable and of satisfactory valency.

When a bond is represented by a line such as “ [symbol] ” this is meant to represent that the bond is the point of attachment between two molecular subunits.

“Mammal” refers to human or animals including livestock and companion animals.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of the invention are also useful as intermediates for preparing other compounds of the invention, a conversion which can occur both in vitro and in vivo.

Some of the compounds of the invention are capable of further forming pharmaceutically acceptable acid-addition and/or base salts. All of these forms are within the scope of the present invention. Thus, pharmaceutically acceptable acid addition salts of the compounds of the invention include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge, S. M. et. al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is an inactive derivative of a drug molecule that requires a chemical or an enzymatic biotransformation in order to release the active parent drug in the body.

Specific and preferred values for the compounds of the present invention are listed below for radicals, substituents, and ranges are for illustration purposes only, and they do not exclude other defined values or other values within defined ranges for the radicals and substituents.

Specifically, alkyl denotes both straight and branched groups; but reference to an individual radical such as “propyl” embraces only the straight chain radical, a branched chain isomer such as “isopropyl” being specifically referred to. Specifically, C₁₋₄ alkyl can be methyl, ethyl, propyl, isopropyl, butyl, iso-butyl, sec-butyl, and their isomeric forms thereof.

Specifically, C₂₋₄ alkenyl can be vinyl, propenyl, allyl, butenyl, and their isomeric forms thereof; C₃₋₆ cycloalkyl can cyclopropyl, cyclobutyl, cyclopentyl, cyclohexyl, and their isomeric forms thereof.

Specifically, halo is fluoro (F), or chloro (Cl).

Specifically, R¹ is C₁₋₄alkyl, optionally substituted with one, two or three fluoro (F), or chloro (Cl).

Specifically, R¹ is CH₃, or CH₂CH₃.

Specifically, R¹ is CHF₂, or CHCl₂.

Specifically, R¹ is CH₂CF₃, or CF₂CH₃.

Specifically, R¹ is H.

Specifically, R¹ is —CH═CH-aryl.

Specifically, R¹ is —CH₂C(═O)C₁₋₄alkyl.

Specifically, R¹ is —CH₂C(═O)aryl.

Specifically, R¹ is CF₃.

Specifically, R¹ is cyclopropyl.

Specifically, R² and R³ are independently H or F.

Specifically, at least one of R² and R³ is F.

Specifically, R² and R³ are F.

Specifically, X is C, or N.

Specifically, L is O or F.

Specifically, n is 1.

Specifically, Y is O or F.

Specifically, m is 1.

Specifically, W is O or F.

Specifically, het¹ is isoxazolyl, isothiazolyl, 1,2,5-thiadiazolyl, or pyridyl.

Specifically, het² is 1,2,3-triazolyl.

Specific compounds of the present invention are those wherein structure i, ii, or iii has an optical configuration as depicted below:

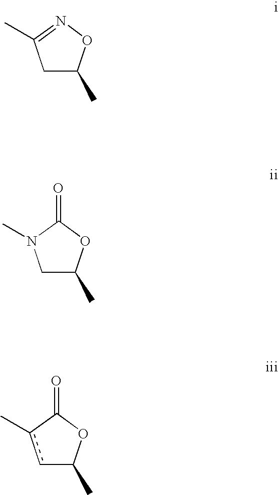

In one embodiment of the invention, a specific structure for 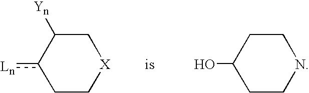
Other specific structures for

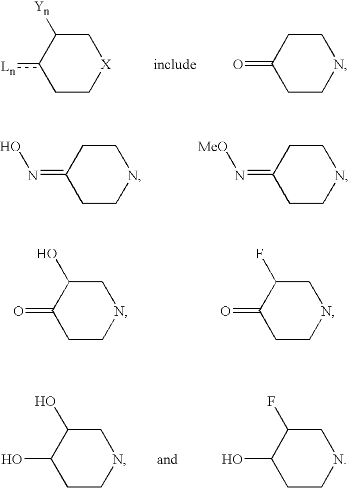

In another embodiment of the invention, a specific structure for 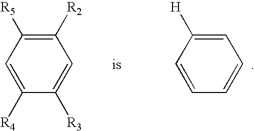
Another specific structure for

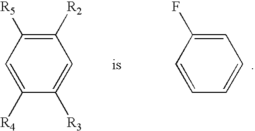

In another embodiment of the invention, a specific structure for 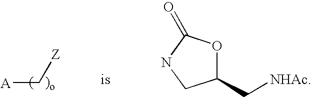
Other specific values for

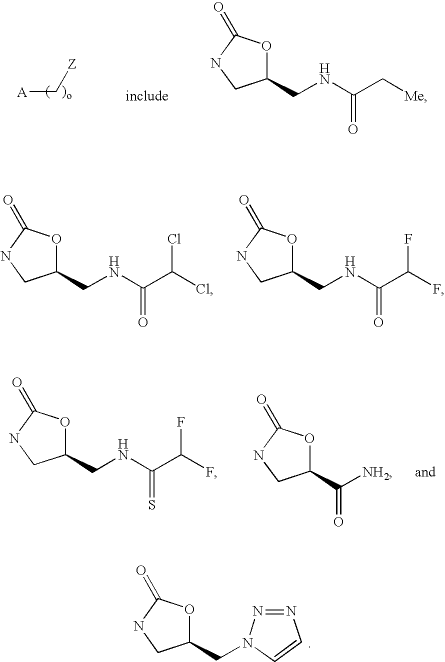

In one group of invention compounds,

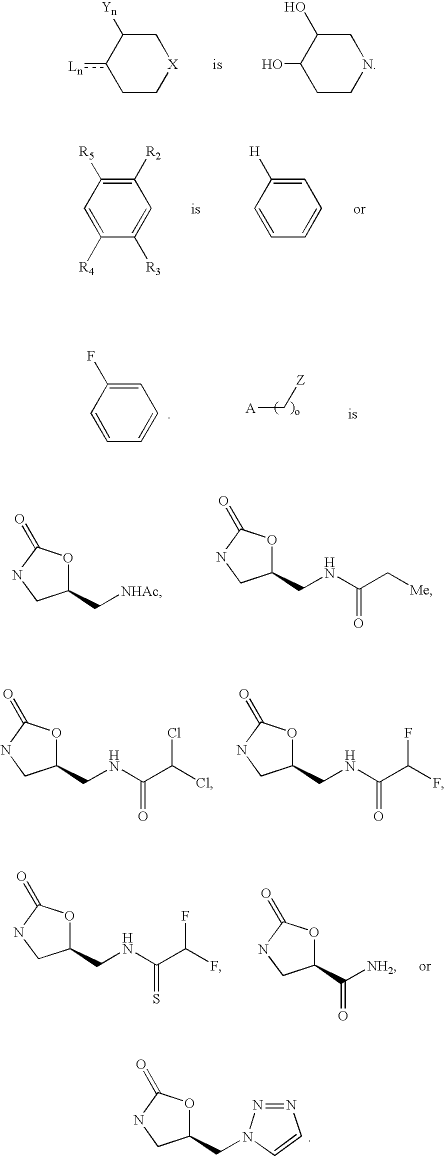

In another group of invention compounds,

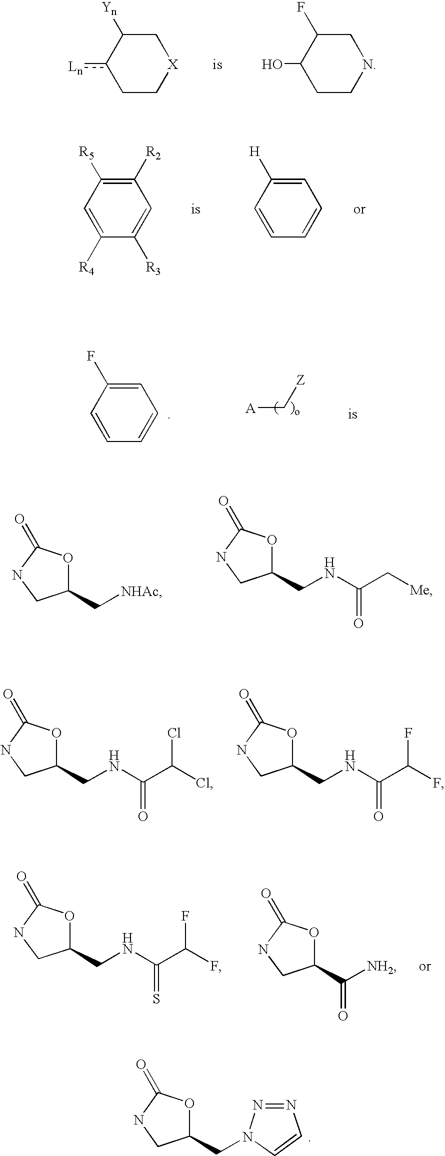

In another group of invention compounds,

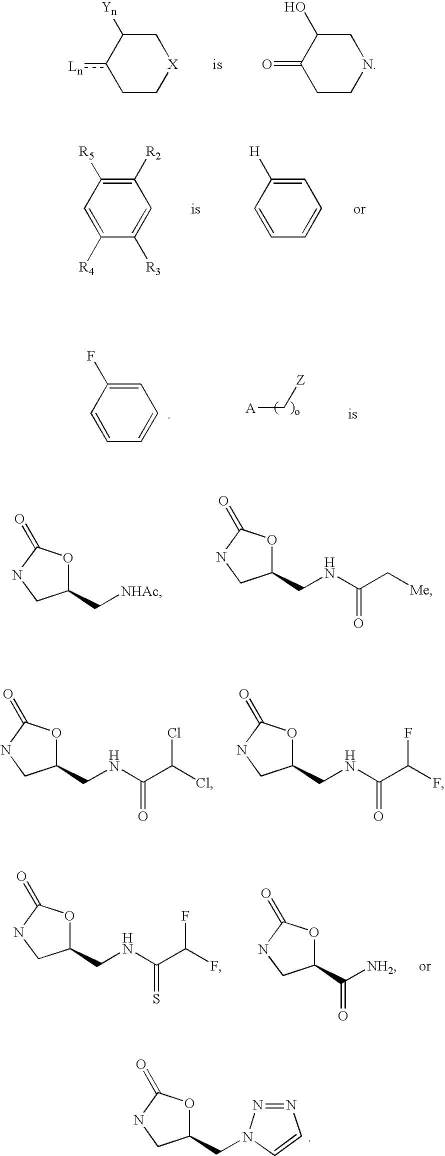

In another group of invention compounds,

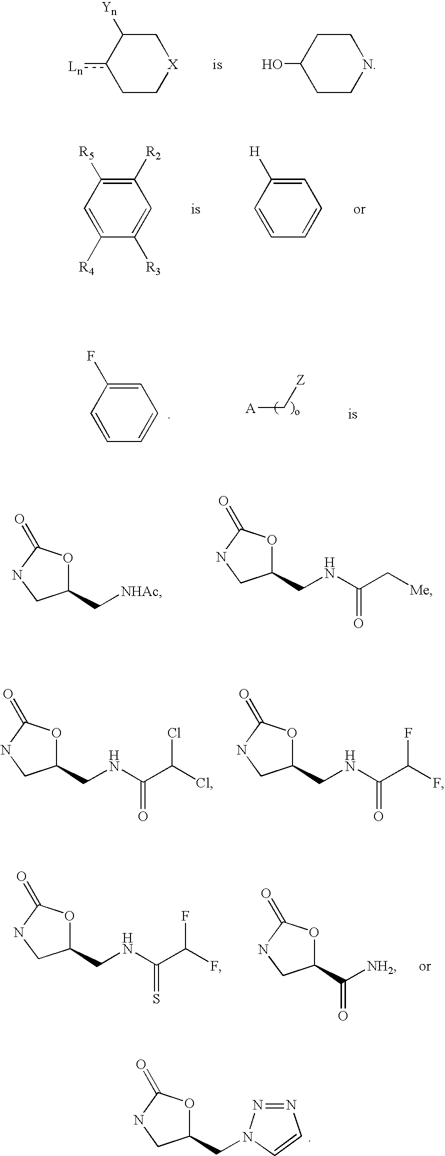

In another group of invention compounds,

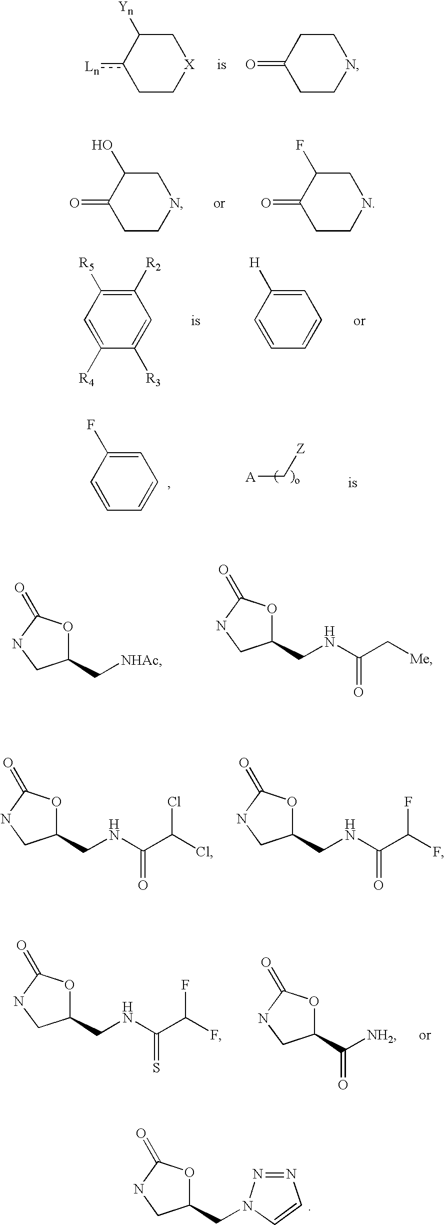

In another group of invention compounds,

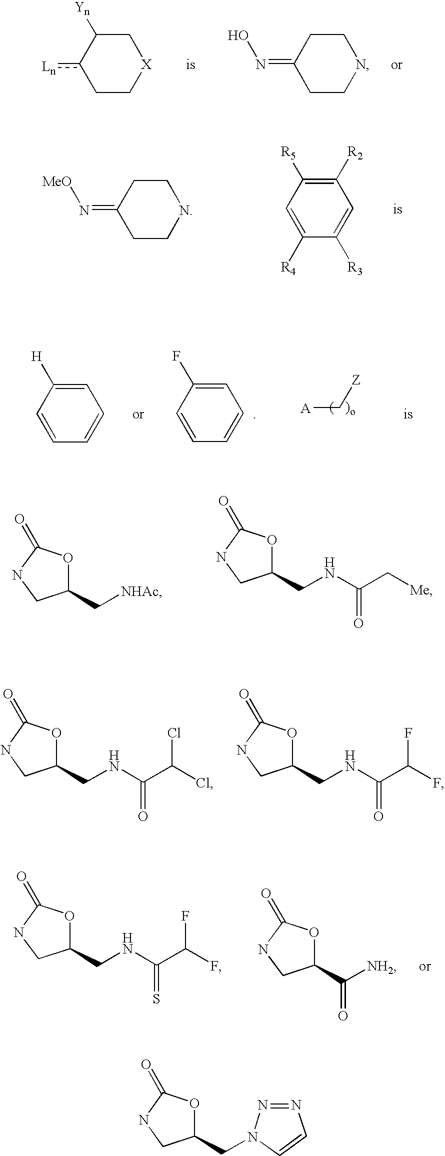

### Preparation of Invention Compounds

Scheme I retrosynthetically depicts an approach to invention compounds such as IA wherein Yₘ is OH and 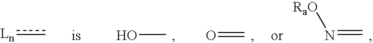
wherein Rₐ is H or (C₁-C₆)alkyl. The compounds can be readily prepared from the corresponding diol. I-B (which is itself an invention compound) using methods readily available to the skilled artisan. Diol I-B can be prepared via vicinal dihydroxylation of alkene I-C. The 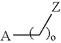
portion of invention compounds can be attached to the aryl nuclus via the amine I-D. Amine I-D can be prepared from the corresponding nitro compound I-E via reduction. I-E can be prepared via general coupling procedures.

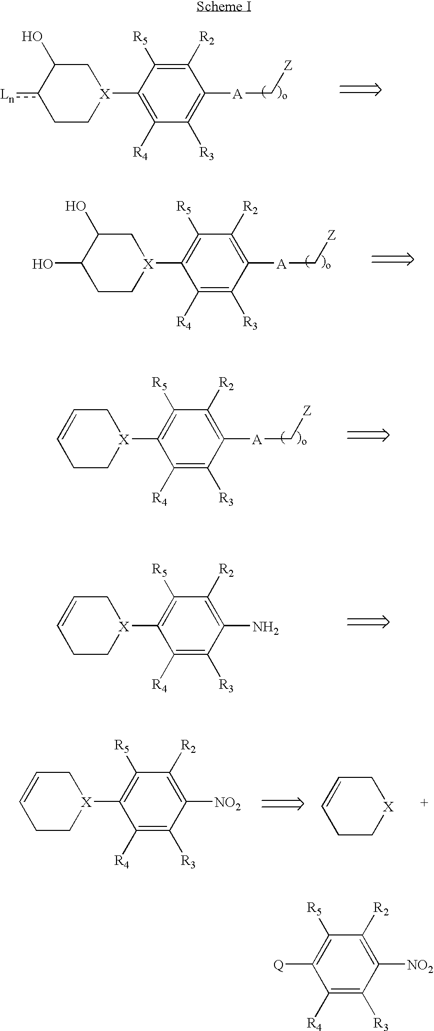

Scheme II retrosynthetically depicts an approach to invention compounds such as IIA wherein Yₘ is H and 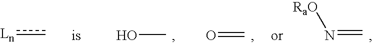
wherein Rₐ is H or (C₁-C₆)alkyl. The approach is similar to that described in Scheme I, except a Diels Alder reactio is used to construct the intermediate

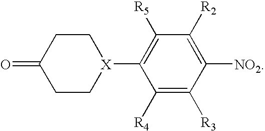

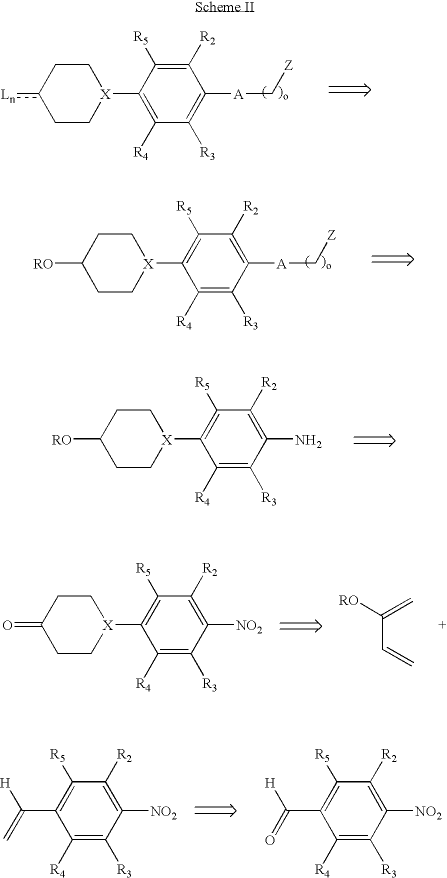

Scheme 1-125 disclose variants of the Scheme I and II approaches. Thus, in Scheme 1, 1,2,3,6-tetrahydropyridine is coupled to 3,4-difluoronitrobenzene in the presence of base to provide 1-(2-Fluoro-4-nitro-phenyl)-1,2,3,6-tetrahydro-pyridine. In this transformation diisopropylethyl amine is used as the base, although other tertiary amine bases commercially available to the skilled artisan may be used, such as triethyl amine, DBU, DBN, and the like. 1-(2-Fluoro-4-nitro-phenyl)-1,2,3,6-tetrahydro-pyridine is converted to 4-(3,6-dihydro-2H-pyridin-1-yl)-3-fluoro-phenylamine upon Fe-mediated reduction, which is subsequently converted to [4-(3,6-dihydro-2H-pyridin-1-yl)-3-fluoro-phenyl]-carbamic acid benzyl ester. [4-(3,6-dihydro-2H-pyridin-1-yl)-3-fluoro-phenyl]-carbamic acid benzyl ester is converted to N-{3-[4-(3,6-Dihydro-2H-pyridin-1-yl)-3-fluoro-phenyl]-2-oxo-oxazolidin-5-ylmethyl}-acetamide upon treatment with (1S)-2-(acetylamino)-1-(chloromethyl)ethyl acetate in the presence of lithium tert-butoxide. OsO4-mediated dihydroxylation of N-{3-[4-(3,6-Dihydro-2H-pyridin-1-yl)-3-fluoro-phenyl]-2-oxo-oxazolidin-5-ylmethyl}-acetamide provides the invention compound, N-{3-[4-(3,4-dihydroxy-piperidin-1-yl)-3-fluoro-phenyl]-2-oxo-oxazolidin-5-ylmethyl}-acetamide.

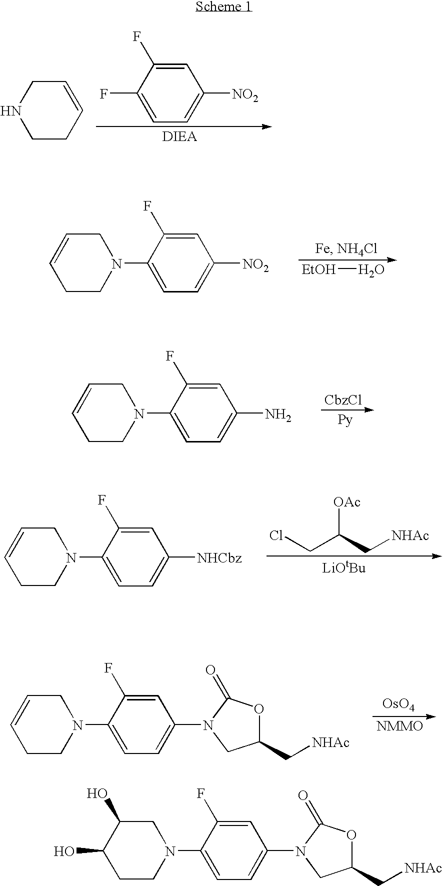

Scheme 2 provides an alternative approach to the synthesis of 1-cyclohex-3-enyl-2-fluoro-4-nitro-benzene, and the invention compound dihydroxy-piperidin-1-yl)-3-fluoro-phenyl]-2-oxo-oxazolidin-5-ylmethyl}-acetamide. Thus, 2-fluoro-4-nitrobenzaldehyde can be converted to the vinyl compound 2-Fluoro-4-nitro-1-vinyl-benzene upon reaction with cyclo-dibromo-di-A-methylene[μ-(tetrahydrofuran)]trizinc (“Nysted Reagent”, CAS No. 41114-59-4) in the presence of a Lewis Acid. Diels Alder reaction of 2-fluoro-4-nitro-1-vinyl-benzene with 2-trimethylsilyloxy-1,3-butadiene provides 4-(2-fluoro-4-nitro-phenyl)-cyclohexanone, which can be converted to 1-cyclohex-3-enyl-2-fluoro-4-nitro-benzene upon reduction and dehydration. Osmium tetroxide mediated dihydroxylation of 1-cyclohex-3-enyl-2-fluoro-4-nitro-benzene provides 4-(2-fluoro-4-nitro-phenyl)-cyclohexane-1,2-diol, which is subsequently protected as the acetonide 5-(2-Fluoro-4-nitro-phenyl)-2,2-dimethyl-hexahydro-benzo[1,3]dioxole. 5-(2-Fluoro-4-nitro-phenyl)-2,2-dimethyl-hexahydro-benzo[1,3]dioxole is reduced using convetional hydrogenation conditions to provide to 4-(2,2-dimethyl-hexahydro-benzo[1,3]dioxol-5-yl)-3-fluoro-phenylamine. Cbz protection of 4-(2,2-dimethyl-hexahydro-benzo[1,3]dioxol-5-yl)-3-fluoro-phenylamine, and subsequent reaction with (1S)-2-(acetylamino)-1-(chloromethyl)ethyl acetate in the presence of base according to Scheme I-A provides the invention compound dihydroxy-piperidin-1-yl)-3-fluoro-phenyl]-2-oxo-oxazolidin-5-ylmethyl}-acetamide. As will be seen in subsequent scheme, the product Diels-Alder adduct 4-(2-fluoro-4-nitro-phenyl)-cyclohexanone and the subsequent compound 4-(2-fluoro-4-nitro-phenyl)-cyclohexanol can be used to access invention compounds other than those depicted in the scheme.

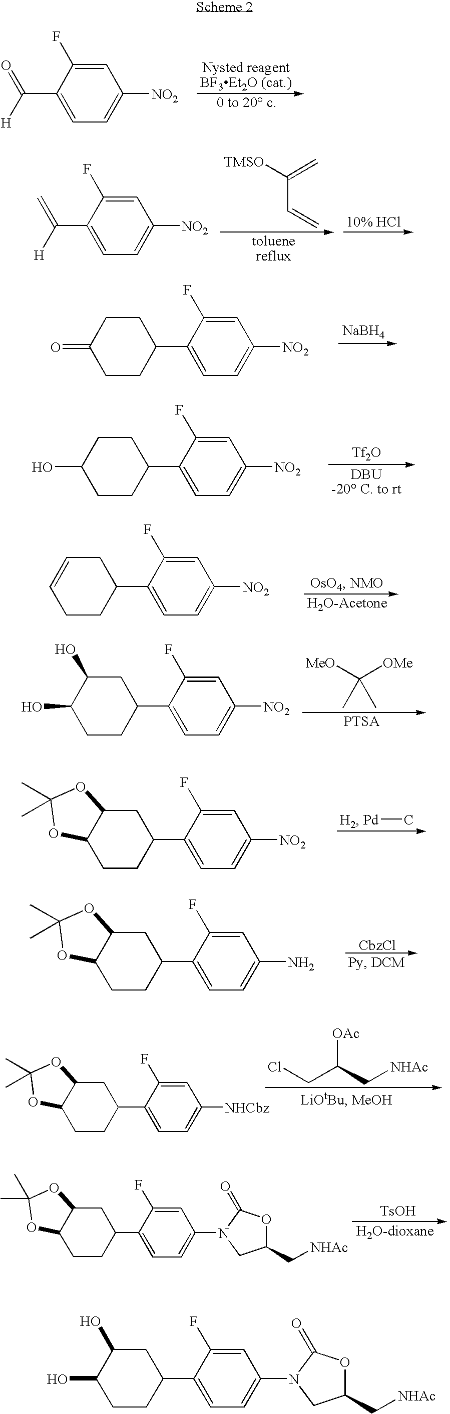

Scheme 3 provides an approach to compounds wherein R₅ is H as opposed to F (as in Schemes 1-2). In Scheme 3,4-nitrostyrene is used for the Diels Alder reaction instead of 2-fluoro-4-nitrostyrene. As will be seen in subsequent scheme, the product Diels-Alder adduct 4-(4-nitro-phenyl)-cyclohexanone and the subsequent compound 4-(4-nitro-phenyl)-cyclohexanol can be used to access invention compounds other than those depicted in the scheme.

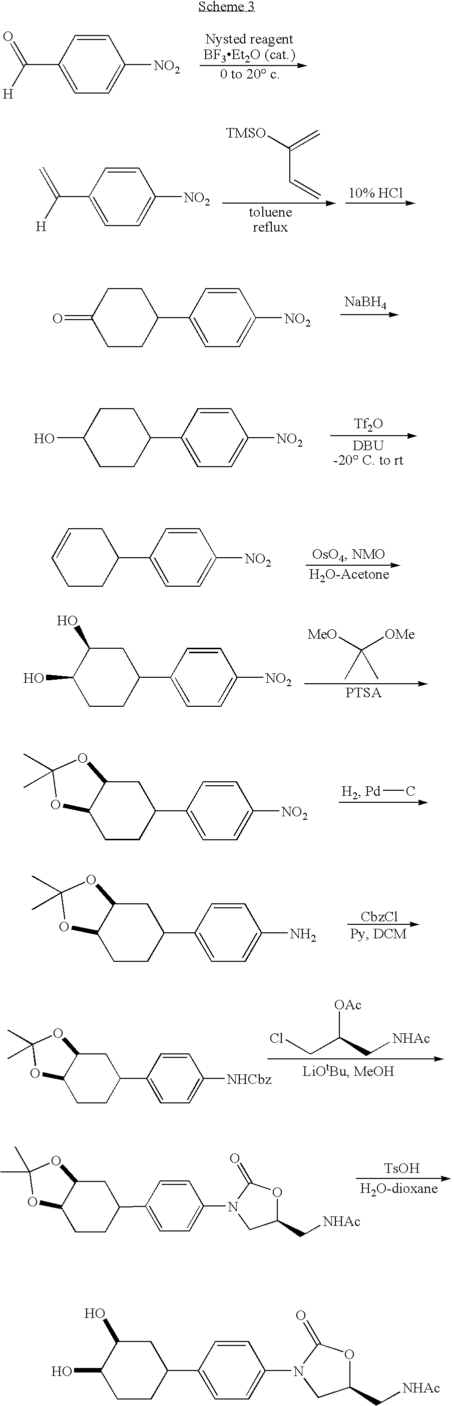

Schemes 4 and 5 depict the tranformation of N-{3-[4-(3,4-Dihydroxy-piperidin-1-yl)-3-fluoro-phenyl]-2-oxo-oxazolidin-5-ylmethyl}-acetamide to other invention compounds. Thus, oxidation of N-{3-[4-(3,4-Dihydroxy-piperidin-1-yl)-3-fluoro-phenyl]-2-oxo-oxazolidin-5-ylmethyl}-acetamide under Swern-type or related conditions provide the corresponding ketone product, N-{3-[3-Fluoro-4-(3-hydroxy-4-oxo-piperidin-1-yl)-phenyl]-2-oxo-oxazolidin-5-ylmethyl}-acetamide.

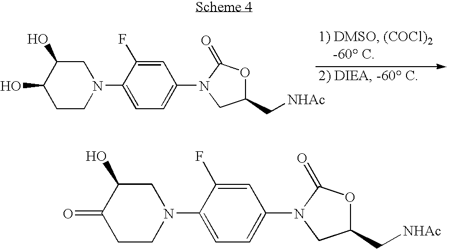

N-{3-[3-Fluoro-4-(3-hydroxy-4-oxo-piperidin-1-yl)-phenyl]-2-oxo-oxazolidin-5-ylmethyl}-acetamide itself can be converted to oxime invention products using established procedures. Thus, treatment of the ketone with hydroxylamine hydrochloride or methoxyamine hydrochloride in the presence of base provides the corresponding oxime N-{3-[3-Fluoro-4-(3-hydroxy-4-hydroxyimino-piperidin-1-yl)-phenyl]-2-oxo-oxazolidin-5-ylmethyl}-acetamide or alkyloxime N-{3-[3-Fluoro-4-(3-hydroxy-4-methoxyimino-piperidin-1-yl)-phenyl]-2-oxo-oxazolidin-5-ylmethyl}-acetamide.

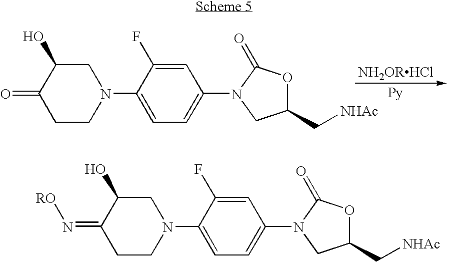

Schemes 6-demonstrate that a variety invention compounds incorporating diverse 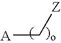
subunits can be prepared using the Scheme I approach. In Scheme I-E, 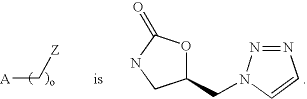
[4-(3,6-dihydro-2H-pyridin-1-yl)-3-fluoro-phenyl]-carbamic acid benzyl ester can undergo reaction with R-(−)-glycidylbutyrate to provide 3-[4-(3,6-dihydro-2H-pyridin-1-yl)-3-fluoro-phenyl]-5-hydroxymethyl-oxazolidin-2-one. Mesylation of 3-[4-(3,6-dihydro-2H-pyridin-1-yl)-3-fluoro-phenyl]-5-hydroxymethyl-oxazolidin-2-one, followed by reaction with sodium azide and cyclopentadiene consecutively, provides 3-[4-(3,6-Dihydro-2H-pyridin-1-yl)-3-fluoro-phenyl]-5-[1,2,3]triazol-1-ylmethyl-oxazolidin-2-one. Osmium tetroxide-mediated dihydroxylation of 3-[4-(3,6-Dihydro-2H-pyridin-1-yl)-3-fluoro-phenyl]-5-[1,2,3]triazol-1-ylmethyl-oxazolidin-2-one provides the invention compound 3-[4-(3,4-Dihydroxy-piperidin-1-yl)-3-fluoro-phenyl]-5-[1,2,3]triazol-1-ylmethyl-oxazolidin-2-one. 3-[4-(3,4-Dihydroxy-piperidin-1-yl)-3-fluoro-phenyl]-5-[1,2,3]triazol-1-ylmethyl-oxazolidin-2-one can be converted to the corresponding ketone or oxime as provided in Schemes I-C or I-D, above.

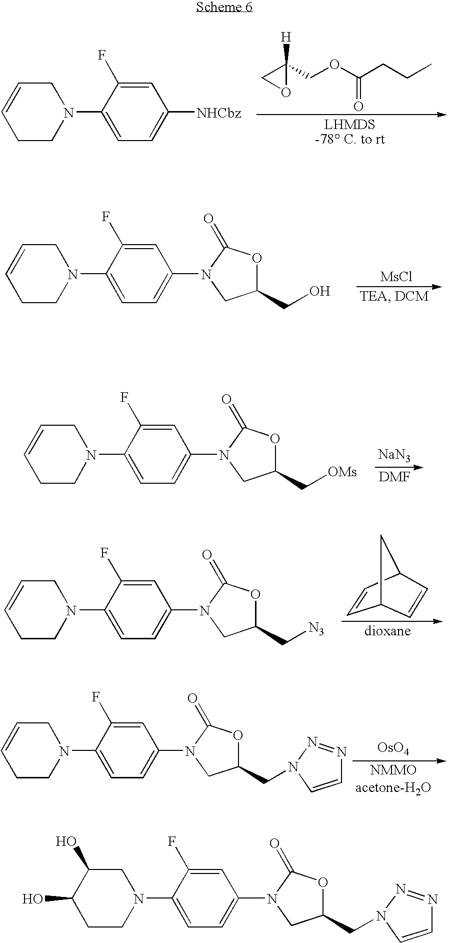

Scheme I-F provides an approach to invention compounds wherein 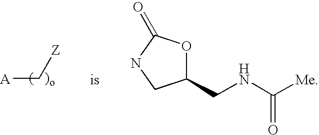
[4-(2,2-dimethyl-hexahydro-benzo[1,3]dioxol-5-yl)-3-fluoro-phenyl]-carbamic acid benzylester (Scheme I-B) undergoes reaction with 1(S)-(tert-butoxycarbonylamino-methyl)-2-chloro-ethyl ester in the presence of base to provide {3-[4-(2,2-dimethyl-hexahydro-benzo[1,3]dioxol-5-yl)-3-fluoro-phenyl]-2-oxo-oxazolidin-5-ylmethyl}-carbamic acid tert-butyl ester. Reaction of {3-[4-(2,2-dimethyl-hexahydro-benzo[1,3]dioxol-5-yl)-3-fluoro-phenyl]-2-oxo-oxazolidin-5-ylmethyl}-carbamic acid tert-butyl ester with HCL in the presence of methanol provides the amine diol, which can be converted to N-{3-[4-(3,4-Dihydroxy-cyclohexyl)-3-fluoro-phenyl]-2-oxo-oxazolidin-5-ylmethyl}-propionamide upon treatment with propionic anhydride.

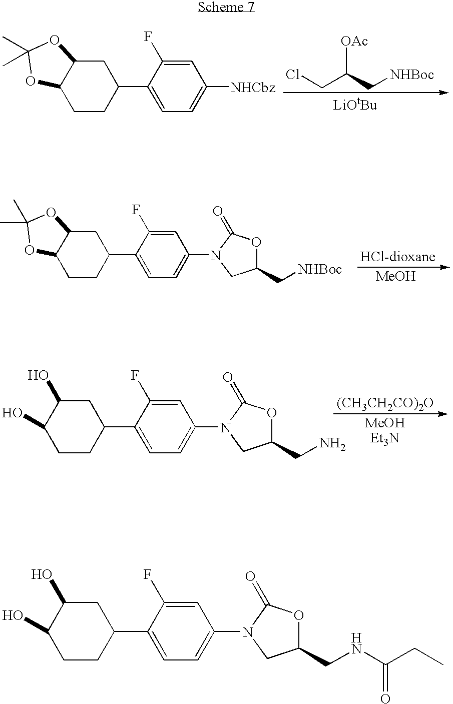

Scheme I-G provides an approach to invention compounds wherein 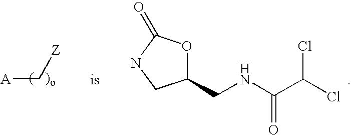
{3-[4-(2,2-dimethyl-hexahydro-benzo[1,3]dioxol-5-yl)-3-fluoro-phenyl]-2-oxo-oxazolidin-5-ylmethyl}-carbamic acid tert-butyl ester (Scheme I-F) is treated with trifluoroacetic acid to provide the amine diol, which was subsequently converted to the invention compound 2,2-Dichloro-N-{3-[4-(3,4-dihydroxy-cyclohexyl)-3-fluoro-phenyl]-2-oxo-oxazolidin-5-ylmethyl}-acetamide upon treatment with ethyl dichloroacetate in the presence of base.

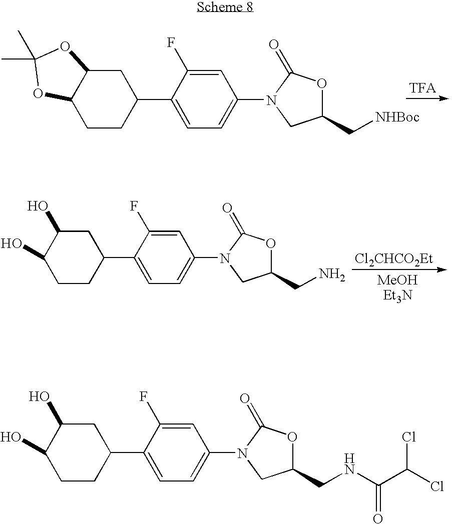

Scheme I-G-1 discloses an approach to the preparation of 2,2-Dichloro-N-{3-[4-(3,4-dihydroxy-cyclohexyl)-phenyl]-2-oxo-oxazolidin-5-ylmethyl}-acetamide in the same manner as provided in Scheme I-G.

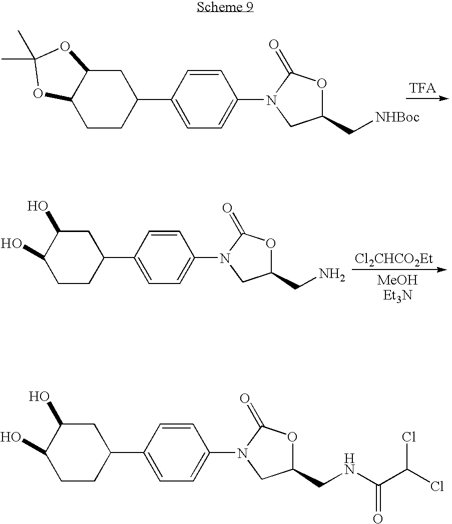

Scheme I-H provides an approach to invention compounds wherein 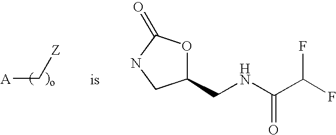
as described for Scheme I-G, except ethyl difluoroacetate is used.

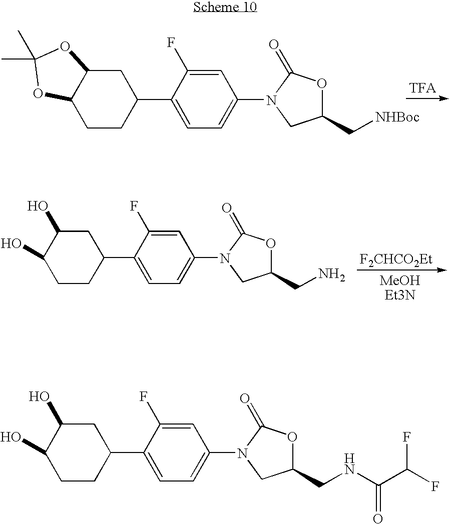

Scheme I-H-1 discloses an approach to the preparation of 2,2-Difluoro-N-{3-[4-(3,4-dihydroxy-cyclohexyl)-phenyl]-2-oxo-oxazolidin-5-ylmethyl}-acetamide in the same manner as provided in Scheme I-H.

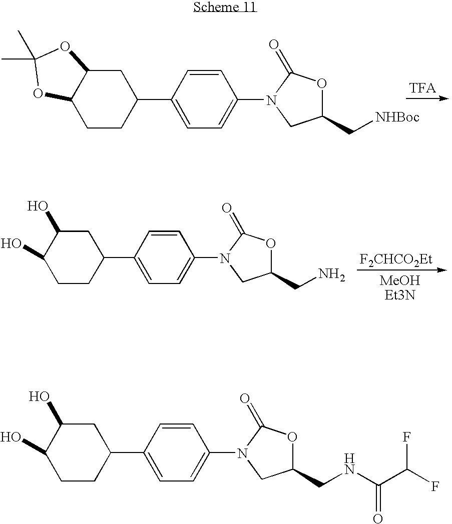

Scheme I-I provides an approach to invention compounds wherein 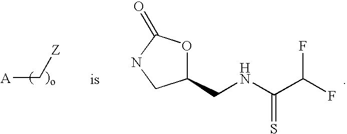
The target compound N-{3-[4-(3,4-Dihydroxy-cyclohexyl)-3-fluoro-phenyl]-2-oxo-oxazolidin-5-ylmethyl}-2,2-difluoro-thioacetamide was prepared in a similar fashion as described in Scheme I-H, except that difluoro-thioacetic acid was used.

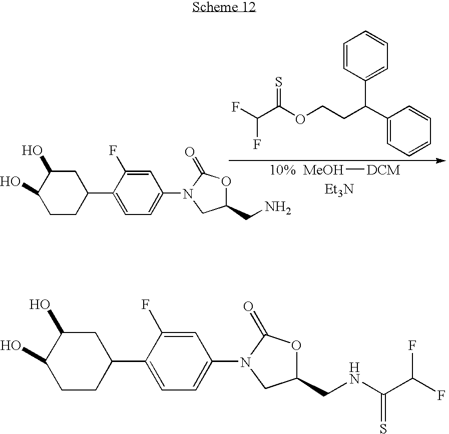

Scheme I-I-1 discloses an approach to the preparation of N-{3-[4-(3,4-Dihydroxy-cyclohexyl)-phenyl]-2-oxo-oxazolidin-5-ylmethyl}-2,2-difluoro-thioacetamide in the same manner as provided in Scheme I-I-1.

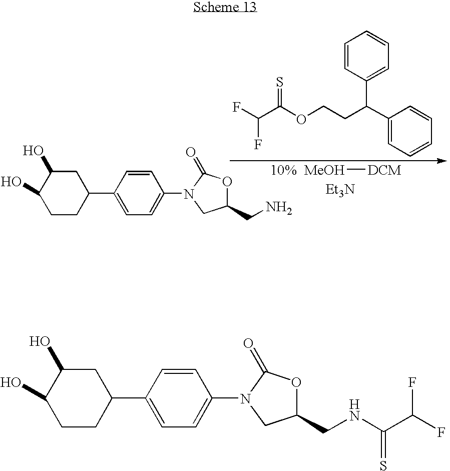

Scheme 14 provides an approach to invention compounds derived from 4-(2-fluoro-4-nitro-phenyl)-cyclohexanol (c.f., Scheme 3). Thus, protection of 4-(2-fluoro-4-nitro-phenyl)-cyclohexanol as the silyl ether provides tert-Butyl-[4-(2-fluoro-4-nitro-phenyl)-cyclohexyloxy]-dimethylsilane. Hydrogenation of silyl ether provides tert-Butyl-[4-(2-fluoro-4-nitro-phenyl)-cyclohexyloxy]-dimethylsilane provides 4-[4-(tert-Butyl-dimethyl-silanyloxy)-cyclohexyl]-3-fluoro-phenylamine.

Completion of the synthesis is as provided in earlier schemes, except that the last step is deprotection of the silylether.

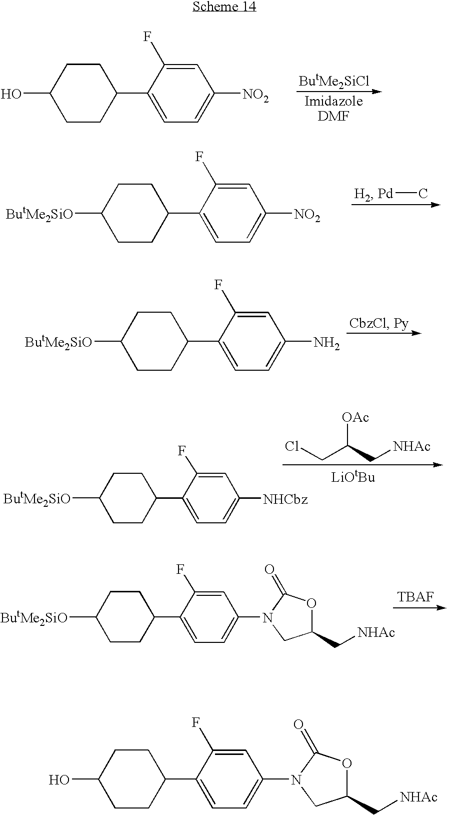

Schemes 17-19 employ 4-[4-(tert-Butyl-dimethyl-silanyloxy)-cyclohexyl]-3-z fluoro-phenylamine to provide compound with alternative 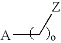
subunits. The approaches are similar to those disclosed in Scheme 5-7.

In Scheme 17,

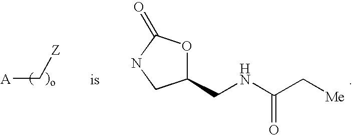

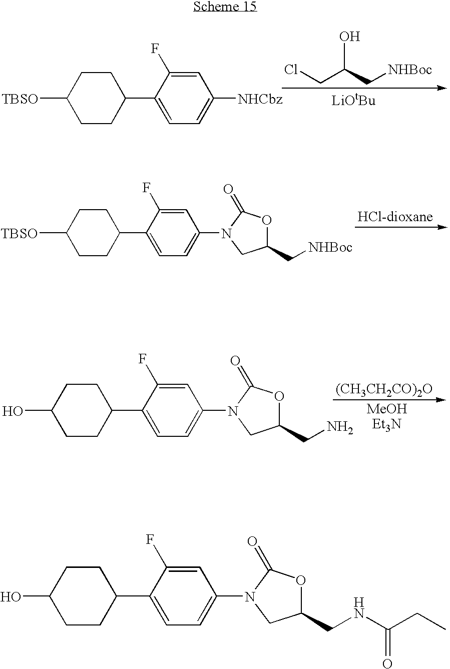

In Scheme 18,

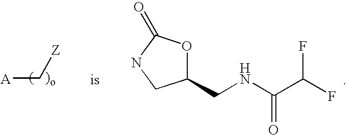

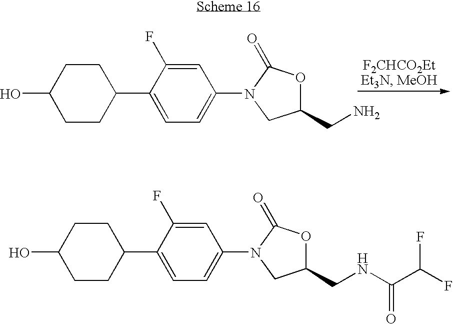

In Scheme 19,

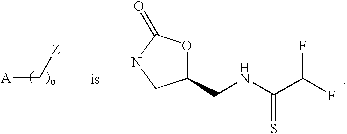

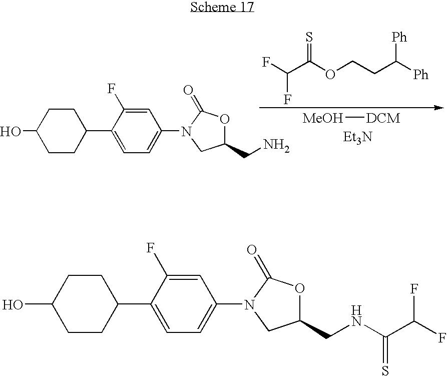

In Scheme 20, the invention compound N-{3-[3-Fluoro-4-(4-hydroxy-cyclohexyl)-phenyl]-2-oxo-oxazolidin-5-ylmethyl}-acetamide (Scheme 14) is oxidized to provide N-{3-[3-Fluoro-4-(4-oxo-cyclohexyl)-phenyl]-2-oxo-oxazolidin-5-ylmethyl}-acetamide.

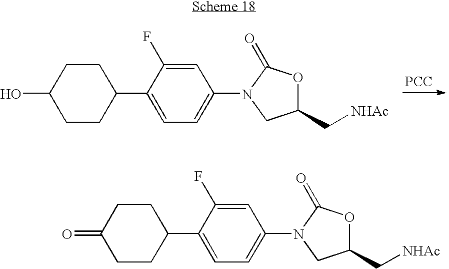

In Scheme 21, N-{3-[3-Fluoro-4-(4-oxo-cyclohexyl)-phenyl]-2-oxo-oxazolidin-5-ylmethyl}-acetamide is converted to the corresponding oxime under conventional conditions.

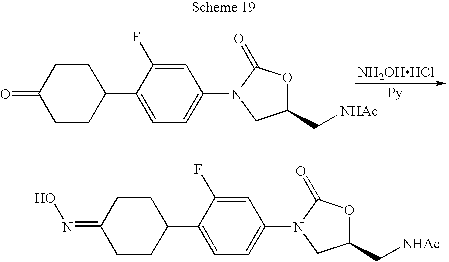

Scheme 22 provides an approach to invention compounds wherein 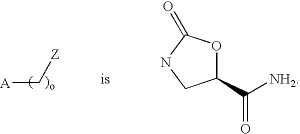
Thus, 4-[4-(tert-Butyl-dimethyl-silanyloxy)-cyclohexyl]-3-fluoro-phenylamine undergoes reaction with (1S)-2-(acetylamino)-1-(chloromethyl)ethyl acetate to provide 3-{4-[4-(tert-butyl-dimethyl-silanyloxy)-cyclohexyl]-3-fluoro-phenylamino}-2-hydroxy-propionic acid ethyl ester. 3-{4-[4-(tert-Butyl-dimethyl-silanyloxy)-cyclohexyl]-3-fluoro-phenylamino}-2-hydroxy-propionic acid ethyl ester is treated with carbonyldiimidazole to provide 3-{4-[4-(tert-Butyl-dimethyl-silanyloxy)-cyclohexyl]-3-fluoro-phenyl}-2-oxo-oxazolidine-5-carboxylic acid ethyl ester and subsequently 3-{4-[4-(tert-Butyl-dimethyl-silanyloxy)-cyclohexyl]-3-fluoro-phenyl}-2-oxo-oxazolidine-5-carboxylic acid amide. Deprotection of 3-{4-[4-(tert-Butyl-dimethyl-silanyloxy)-cyclohexyl]-3-fluoro-phenyl}-2-oxo-oxazolidine-5-carboxylic acid amide provides 3-[3-Fluoro-4-(4-hydroxy-cyclohexyl)-phenyl]-2-oxo-oxazolidine-5-carboxylic acid amide.

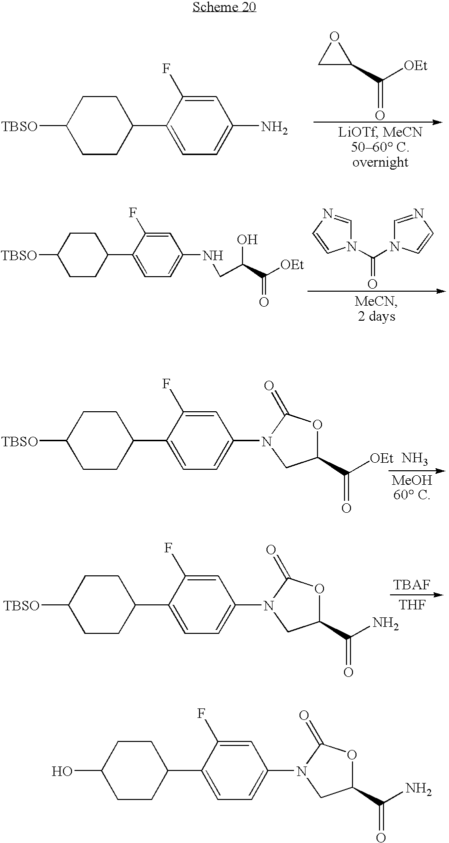

In Scheme 23, 3-[3-Fluoro-4-(4-hydroxy-cyclohexyl)-phenyl]-2-oxo-oxazolidine-5-carboxylic acid amide is oxidized to provide 3-[3-Fluoro-4-(4-oxo-cyclohexyl)-phenyl]-2-oxo-oxazolidine-5-carboxylic acid amide.

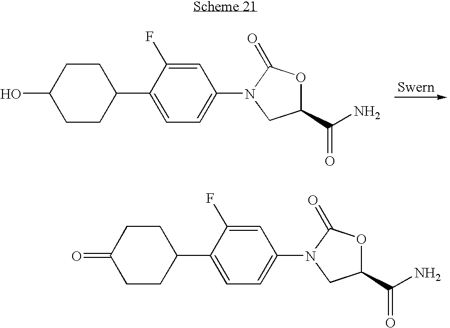

Scheme 24 discloses the synthesis of N-{3-[3-Fluoro-4-(3-hydroxy-4-oxo-cyclohexyl)-phenyl]-2-oxo-oxazolidin-5-ylmethyl}-acetamide. 3-[3-Fluoro-4-(4-oxo-cyclohexyl)-phenyl]-2-oxo-oxazolidine-5-carboxylic acid amide (Scheme 23) is converted to the silylenol ether. The silolenol ether was used without purification and hydroxylated using osmium tetroxide to provide 4-(2-Fluoro-4-nitro-phenyl)-2-hydroxy-cyclohexanone. Protection of the ether moiety in 4-(2-Fluoro-4-nitro-phenyl)-2-hydroxy-cyclohexanone provides 2-(tert-Butyl-dimethyl-silanyloxy)₄-(2-z fluoro-4-nitro-phenyl)-cyclohexanone. The 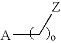
subunit is attached to 2-(tert-Butyl-dimethyl-silanyloxy)-4-(2-fluoro-4-nitro-phenyl)-cyclohexanone as provided in earlier schemes (e.g., reduction of the nitro group to the amine; protection of the ketone moiety; construction if the 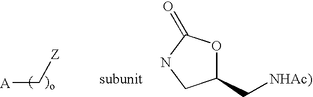
to provide the target compound.

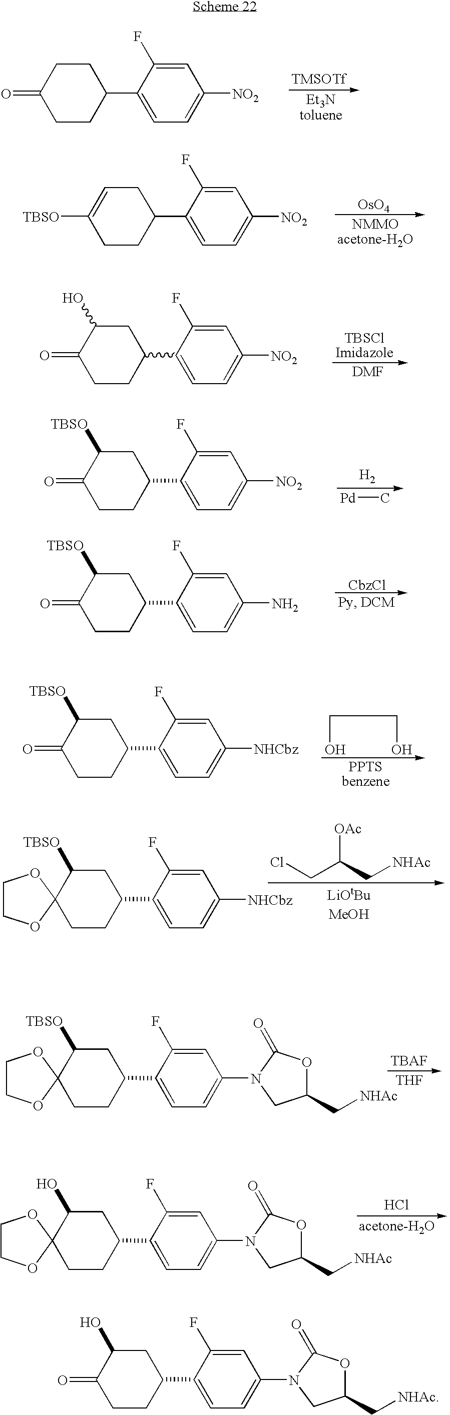

Scheme 25 discloses the synthesis of N-{3-[3-Fluoro-4-(3-fluoro-4-hydroxy-cyclohexyl)-phenyl]-2-oxo-oxazolidin-5-ylmethyl}-acetamide. As in Scheme 24, 3-[3-Fluoro-4-(4-oxo-cyclohexyl)-phenyl]-2-oxo-oxazolidine-5-carboxylic acid amide (Scheme 23) is converted to the silylenol ether. The silylenol ether was treated with Selectfluor® 1-Chloromethyl-4-Fluoro-1,4-Diazoniabicyclo[2.2.2]Octane Bis-(Tetrafluoroborate) (Air Products, http://www.airproducts.com/index.asp last visited Aspril 10, 2004) to provide 2-Fluoro-4-(2-fluoro-4-nitro-phenyl)-cyclohexanone. The 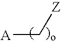
subunit is attached to 2-Fluoro-4-(2-fluoro-4-nitro-phenyl)-cyclohexanone as provided in Scheme 24 (e.g., reduction of the nitro group to the amine; protection of the ketone moiety; construction if the 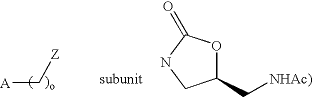
to provide the target compound.

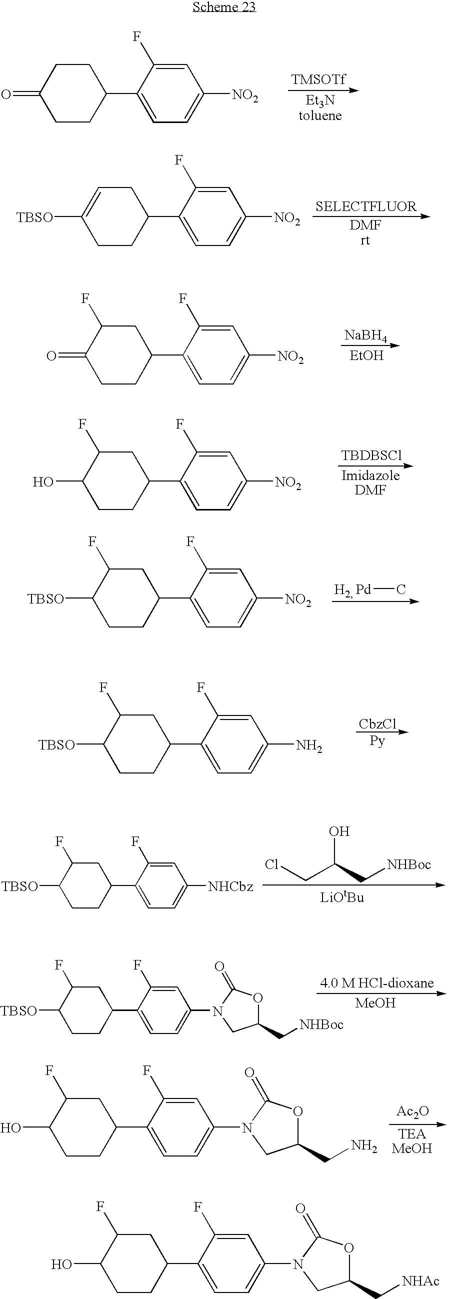

Scheme 26 provides an approach to an additional invention compound wherein 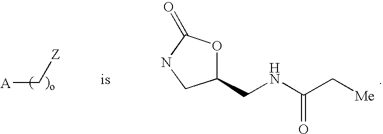
The approach is as provided for Scheme 8.

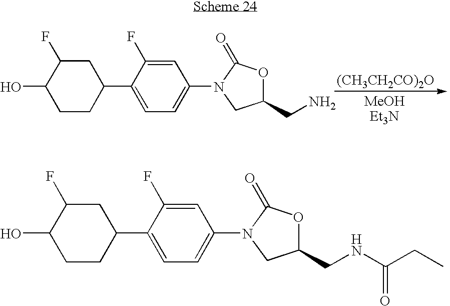

Scheme 27 provides an approach to an additional invention compound wherein 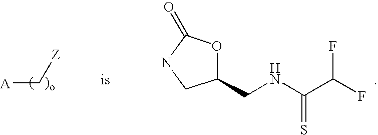
The approach is as provided for Scheme 19.

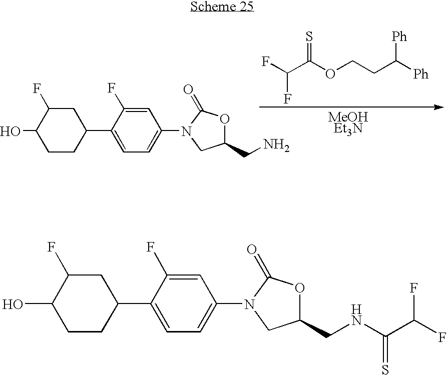

### Pharmaceutical Formulations

The present invention also provides pharmaceutical compositions which comprise a bioactive invention compound or a salt such as a pharmaceutically acceptable salt thereof and optionally a pharmaceutically acceptable carrier. The compositions include those in a form adapted for oral, topical or parenteral use and can be used for the treatment of bacterial infection in mammals including humans.

The compounds, such as antibiotic compounds, also referred to herein as antimicrobial compounds, according to the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

### Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing an infectious disorder in a subject, such as a human or other animal subject, are provided, by administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985;28:766; Heifetz, et. al., Antimicrob., 1974;6:124). The results of the evaluation are shown in Tables 1A and B.

TABLE 1
Antibacterial Activity Minimum Inhibitory Concentration (μg/mL)

| | SAUR 9213 | SPNE 9912 | HINF 30063 | MC 30603 |
|---|---|---|---|---|
| EXAMPLE # | MIC | MIC | MIC | MIC |
| 1 | 8 | 2 | 32 | 8 |
| 2 | 4 | 1 | 16 | 8 |
| 3 | 8 | 2 | 16 | 16 |
| 4 | 8 | 2 | 64 | 32 |
| 5 | 4 | 1 | 32 | 16 |
| 6 | 4 | 1 | 64 | 8 |
| 7 | 4 | 0.5 | 8 | 4 |
| 8 | 8 | 2 | 8 | 8 |
| 9 | 4 | 1 | 4 | 4 |
| 10 | 4 | 1 | 8 | 4 |
| 11 | 1 | 0.5 | 8 | 4 |
| 12 | 16 | 4 | 16 | 16 |
| 13 | 8 | 1 | 8 | 8 |
| 14 | 8 | 2 | 16 | 16 |
| 15 | 4 | 1 | 16 | 8 |
| 16 | 2 | 1 | 8 | 4 |
| 17 | 2 | 1 | 4 | 4 |
| 18 | 2 | 0.5 | 8 | 4 |
| 19 | 2 | 0.5 | 8 | 2 |
| 20 | 2 | 0.5 | 16 | 8 |
| 21 | 1 | 0.5 | 16 | 8 |
| 22 | 2 | 1 | 32 | 1 |
| 23 | 2 | 1 | 64 | 16 |
| 24 | 1 | 2 | 32 | 32 |
| 25 | 2 | 2 | 16 | 8 |
| 26 | 2 | 0.5 | 16 | 8 |
| 27 | 2 | 0.25 | 8 | 2 |
| 28 | 1 | 0.5 | 32 | 2 |

### EXAMPLES

The following examples are provided to illustrate but not limit the claimed invention.

### Example 1

### Preparation of N-{3-[4-(3,4-Dihydroxy-cyclohexyl)-3-fluoro-phenyl]-2-oxo-oxazolidin-5-yl}-acetamide

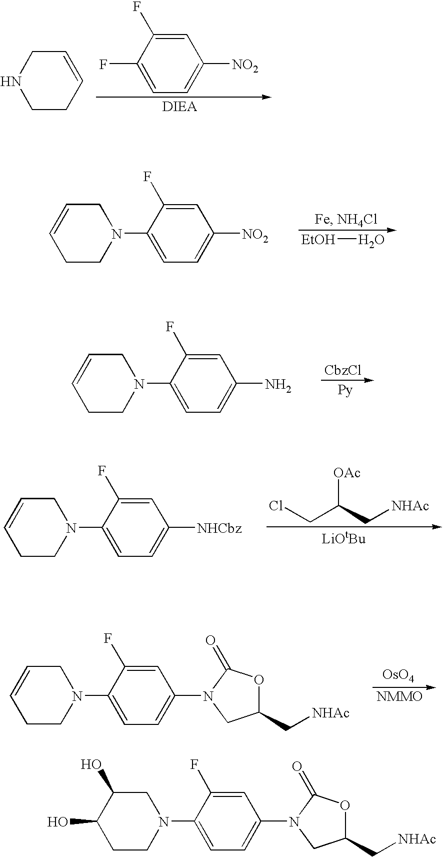 1-(2-Fluoro-4-nitro-lphenyl)-1,2,3,6-tetrahydro-pyridine

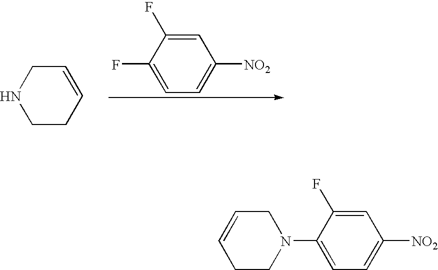

1,2,3,6-Tetrahydropyridine (0.5 g, 6.01 mmol) was dissolved in 3 mL of DMF. 3,4-difluoronitrobenzene (0.8 mL, 7.22 mmol) was then added to the reaction mixture, followed by N,N-diisopropylethylamine (2.1 mL, 12.02 mmol). The reaction mixture was then heated at 50° C. for 3 hours. Solvent was removed under reduced pressure and the residue was taken up in ethyl acetate, washed with brine. The organic layer was dried over MgSO₄. Solvent was removed and the residue was rinsed with hexanes. The resulting oil solidified to give 1.21 g (91%) of the desired product as a pale yellow solid. HPLC: retention time 5.40 minutes, purity >99%.
4-(3,6-Dihydro-2H-pyridin-1-yl)-3-fluoro-phenylamine

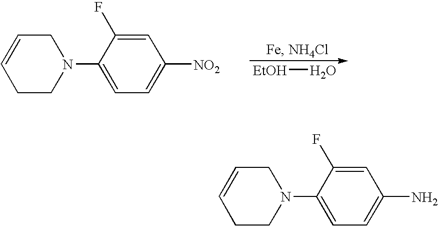

1-(2-Fluoro-4-nitro-phenyl)-1,2,3,6-tetrahydro-pyridine (4.4 g, 19.64 mmol) was dissolved in 30 mL of ethanol-water (2:1) solution. Then ammonium chloride (10.5 g, 0.2 mol) was added and the reaction mixture was heated to 100° C. During this time iron power (3.3 g, 58.92 mmol) was added to the reaction mixture in three portions. After heating for 3 hours at 100° C., the mixture was cooled to room temperature and filtered. The solution was then extracted with ethyl acetate, and the organic layers were combined and washed with brine, dried over MgSO₄. Solvent was removed to give the desired product as an oil (3.5 g, 92%). HPLC: retention time 2.62 minutes, purity >99%; [M+H]⁺ 193.3.
[4-(3,6-Dihydro-2H-pyridin-1-yl)-3-fluoro-phenyl]-carbamic acid benzyl ester

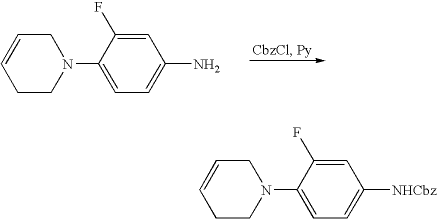

Benzyl chloroformate (2.2 mL, 15.19 mmol) was added dropwise to a mixture of 4-(3,6-dihydro-2H-pyridin-1-yl)-3-fluoro-phenylamine (2.4 g, 12.66 mmol) and pyridine (2.5 mL, 30.38 mmol) in dichloromethane (30 mL) at 0° C. The reaction mixture was stirred for 30 minutes at 0° C., and then warmed up to room temperature. The reaction mixture was poured into water, extracted with EtOAc, and the organic layer was separated, washed with brine, and dried over MgSO₄. The solvent was then removed and the residue was purified by preparative TLC (25% EtOAc/hexanes). The desired product was obtained as a white solid (3.8 g, 92%). HPLC: retention time 4.28 minutes, purity >99%; [M+H]⁺ 327.3.
N-{3-[4-(3,6-Dihydro-2H-pyridin-1-yl)-3-fluoro-phenyl]-2-oxo-oxazolidin-5-ylmethyl}-acetamide

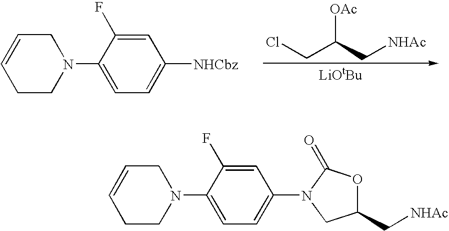

To a solution of [4-(3,6-dihydro-2H-pyridin-1-yl)-3-fluoro-phenyl]-carbamic acid benzyl ester (300 mg, 0.92 mmol) in DMF (1.5 mL) and methanol (75 μL, 1.86 mmol) was added a solution of lithium tert-butoxide (1.0 M solution in THF, 2.8 mL, 2.76 mmol) at room temperature. The solution was then cooled to 0° C. and (1S)-2-(acetylamino)-1-(chloromethyl)ethyl acetate (356 mg, 1.84 mmol) was added. The reaction was warmed up to room temperature and stirred overnight. Saturated aqueous ammonium chloride (2 mL) was added followed by water (20 mL). The reaction mixture was extracted with dichloromethane, washed with brine, and the organic layer was dried over MgSO₄. Solvent was removed and the residue was purified by preparative TLC (10% methanol/dichloromethane) to give the product as a white solid (250 mg, 82%). HPLC: R.T. 3.16 minutes, purity >99%; [M+Na]⁺ 356.3.
N-{3-[4-(3,4-Dihydroxy-piperidin-1-yl)-3-fluoro-phenyl]-2-oxo-oxazolidin-5-ylmethyl}-acetamide

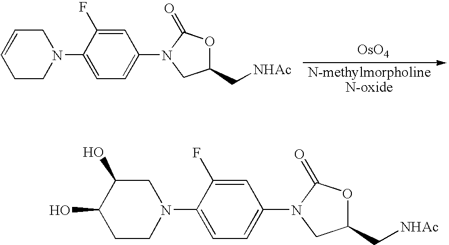

N-{3-[4-(3,6-Dihydro-2H-pyridin-1-yl)-3-fluoro-phenyl]-2-oxo-oxazolidin-5-ylmethyl}-acetamide (250 mg, 0.75 mmol) was dissolved in 5 mL of acetone-water (4:1) solution. Then 4-methylmorpholine N-oxide (NMMO, 193 mg, 1.65 mmol) was added followed by osmium tetroxide (2.5 wt. % solution in Bu^{t}OH, 50 μL). The reaction was stirred overnight, concentrated, and purified by preparative TLC (10% methanol/dichloromethane). The product was collected as a white solid (260 mg, 94%). ¹H NMR (300 MHz, DMSO) δ 8.23 (t, J=5.8 Hz, 1H), 7.45 (dd, J=14.8 Hz, 1.8 Hz, 1H), 7.16-7.04 (m, 2H), 4.72-4.58 (m, 1H), 4.06 (t, J=9.0 Hz, 2H), 3.76-3.62 (m, 4H), 3.38 (t, J=5.7 Hz, 2H), 3.02-2.83 (m, 4H), 1.82-1.65 (m, 5H); HPLC: R.T. 2.82 minutes, purity >95%; [M+H]⁺ 368.3.

### Example 1A

### Alternative Preparation of N-{3-[4-(3,4-Dihydroxy-cyclohexyl)-3-fluoro-phenyl]-2-oxo-oxazolidin-5-ylmethyl}-acetamide

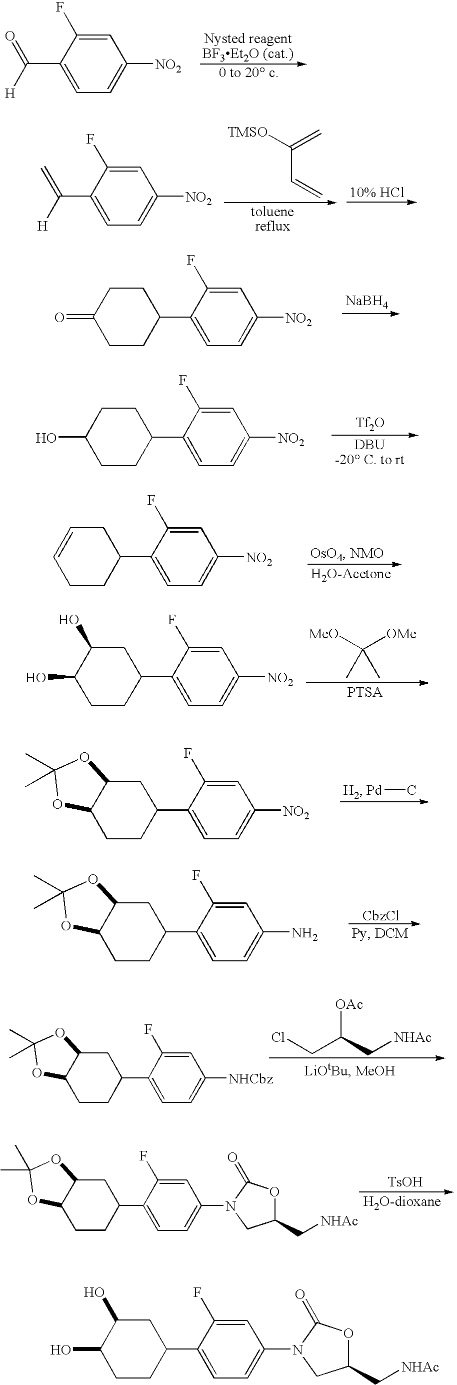 2-Fluoro-4-nitro-1-vinyl-benzene

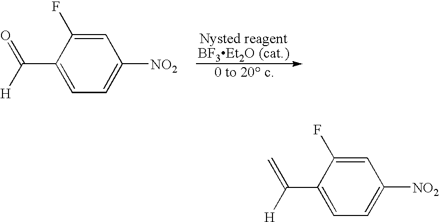

Under N₂ atmosphere, Nysted reagent (20 wt. % suspension in THF, 2 mL, 1.0 mmol) and BF₃.Et₂O (13 μL, 0.1 mmol) were mixed together with 3 mL of THF at 0° C. A solution of 2-fluoro-4-nitrobenzaldehyde (169 mg, 1.0 mmol) in THF was then added at 0° C. The reaction mixture was warmed up to room temperature and stirred for another 2 hours. During this time the precipitate (Nysted reagent) disappeared and the solution turned to be yellow-brown. The resulting mixture was poured into 1.0 M HCl solution and extracted with EtOAc. The organic layers were combined and washed with brine, dried over MgSO₄ and concentrated. Purification by column chromatography (20% EtOAc/hexanes) gave 130 mg (78%) of desired product as a yellow liquid. HPLC: retention time 4.87 minutes, Purity >99%.
4-(2-Fluoro-4-nitro-phenyl)-cyclohexanone

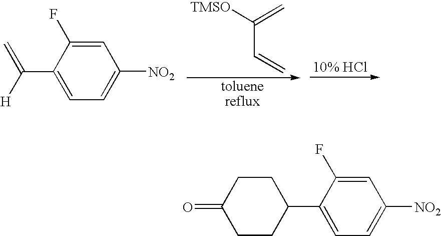

2-Fluoro-4-nitro-1-vinyl-benzene (2.0 g, 12.0 mmol) and 2-trimethylsilyloxy-1,3-butadiene (5.0 mL, 28.8 mmol) were mixed with 15.0 mL of anhydrous toluene. The mixture was placed in a sealed tube and heated to refluxing for 48 hours. The reaction mixture was then cooled to room temperature and poured into 10% HCl. The solution was then stirred for 1 hour at room temperature and extracted with EtOAc. The combined organic layers were washed with diluted NaHCO₃, brine and dried over MgSO₄. The solvent was removed and the residue was purified by column chromatography (30% EtOAc/hexanes). The product was obtained as a pale yellow solid (1.6 g, 56%). HPLC: retention time 4.85 minutes, Purity >95%.
1-Cyclohex-3-enyl-2-fluoro-4-nitro-benzene

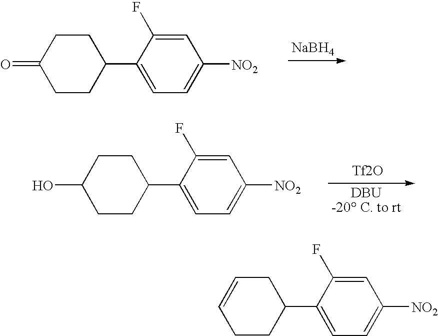

To a solution of 4-(2-Fluoro-4-nitro-phenyl)-cyclohexanone (1.0 g, 4.2 mmol) in 10 mL of absolute ethanol was added 319 mg (8.4 mmol) of NaBH. The solution was then stirred for 2 hours at room temperature, and quenched carefully with 10% HCl solution. The reaction mixture was then extracted with EtOAc, and the combined organic layers were washed with brine and dried over MgSO₄. Solvent was removed under vacuum and the alcohol (567 mg, 56%) was used directly for the next step without further purification.

The alcohol (567 mg, 2.4 mmol) was dissolved in 10 mL of dichloromethane, and the solution was cooled to −20° C. DBU (851 μL, 5.7 mmol) was then added followed by trifluoromethanesulfonic anhydride (479/L, 2.8 mmol). The reaction mixture was then warmed up to room temperature and quenched with water, extracted with EtOAc, and the organic layer was washed with brine. The solvent was removed under vacuum, and the residue was purified by column chromatography (20% EtOAc/hexanes) to give the desired product (458 mg, 87%) as a yellow oil. HPLC: retention time 5.65 minutes, Purity >95%.
4-(2-Fluoro-4-nitro-phenyl)-cyclohexane-1,2-diol

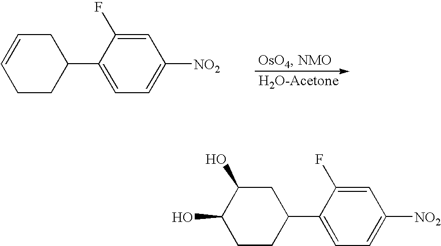

1-Cyclohex-3-enyl-2-fluoro-4-nitro-benzene (458 mg, 2.1 mmol) was dissolved in 5 mL of acetone-water (4:1) solution. Then NMMO (486 mg, 4.1 mmol) was added followed by osmium tetroxide (2.5 wt. % solution in Bu^{t}OH, 90 μL). The reaction was stirred overnight, concentrated, and purified by preparative TLC (20% EtOAc/hexanes). The product was collected as a white solid (458 mg, 87%). HPLC: retention time 3.72 min (mixture of two diastereomers), Purity >95%.
5-(2-Fluoro-4-nitro-phenyl)-2,2-dimethyl-hexahydro-benzo[1,3]dioxole

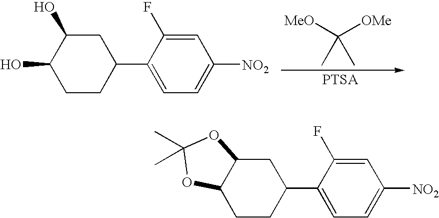

To a solution of 4-(2-fluoro-4-nitro-phenyl)-cyclohexane-1,2-diol (458 mg, 1.8 mmol) in 2.0 mL of 2,2-dimethoxypropane was added catalytic amount of p-toluenesulfonic acid monohydrate (PTSA). The reaction was then stirred at room temperature for 2 hours. Solvent was removed under vacuum and the residue was purified by preparative TLC (17% EtOAc/hexanes). The TLC plates were developed twice and the two diastereomers were separated very carefully. Both the upper spot (diastereomer-1, 180 mg) and the lower spot (diastereomer-2, 290 mg) were obtained as pale yellow solids.
Diastereomer-1: HPLC: retention time 5.13 minutes, Purity >95%.
Diastereomer-2: HPLC: retention time 5.15 minutes, Purity >95%.
   4-(2,2-Dimethyl-hexahydro-benzo[1,3]dioxol-5-yl)-3-fluoro-phenylamine

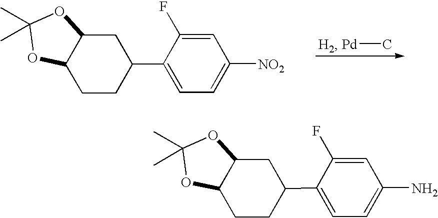

5-(2-Fluoro-4-nitro-phenyl)-2,2-dimethyl-hexahydro-benzo[1,3]dioxole (diastereomer-1, 180 mg, 0.6 mmol) was dissolved in 10 mL of MeOH, and 20 mg of Pd—C (5 wt. % on activated carbon) catalyst was added to the solution. A balloon filled with hydrogen was placed on top of the flask and the reaction mixture was stirred overnight at room temperature. The solution was then passed through a short celite pad and washed with methanol. The collected solution was condensed under vacuum and the residue was dried under high vacuum. The desired product was obtained as a viscous liquid (154 mg, 95%). HPLC: R.T. 3.21 minutes, purity >95%; [M+H]⁺ 266.5.

Diastereomer-2 (290 mg, 0.98 mmol) was carried out in a similar manner and the product was obtained as a glassy solid (247 mg, 95%). HPLC: R.T. 3.12 minutes, purity >95%; [M+H]⁺ 266.5.
[4-(2,2-Dimethyl-hexahydro-benzo[1,3]dioxol-5-yl)-3-fluoro-phenyl]-carbamic acid benzylester

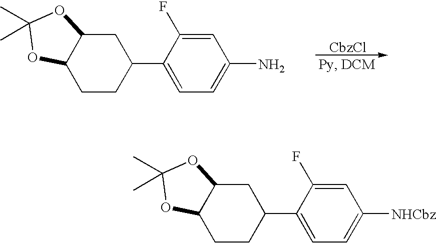

Benzyl chloroformate (110 μL, 0.77 mmol) was added dropwise to a mixture of 4-(2,2-dimethyl-hexahydro-benzo[1,3]dioxol-5-yl)-3-fluoro-phenylamine (diastereomer-1, 170 mg, 0.64 mmol) and pyridine (124 μL, 1.54 mmol) in DCM (10 mL) at 0° C. The reaction mixture was stirred for 30 minutes at 0° C., and then warmed up to room temperature. The reaction mixture was poured into water, extracted with EtOAc, and the organic layer was separated, washed with brine and dried over MgSO₄. The solvent was then removed and the residue was purified by preparative TLC (25% EtOAc/hexanes). The desired product was obtained as a foaming solid (236 mg, 92%). HPLC: R.T. 5.52 minutes, purity >95%; [M+Na]⁺ 422.6.

Diastereomer-2 (283 mg, 0.71 mmol) was carried out in a similar manner and the product was obtained as a foaming solid (350 mg, 82%). HPLC: R.T. 5.53 minutes, purity >95%; [M+Na]⁺ 422.7.
N-{3-[4-(2,2-Dimethyl-hexahydro-benzo[1,3]dioxol-5-yl)-3-fluoro-phenyl]-2-oxo-oxazolidin-5-ylmethyl}-acetamide

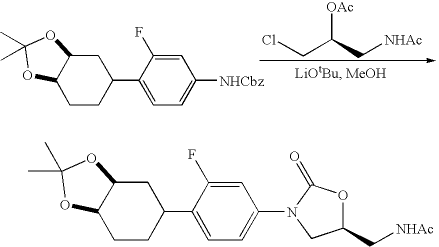

To a solution of [4-(2,2-dimethyl-hexahydro-benzo[1,3]dioxol-5-yl)-3-fluoro-phenyl]-carbamic acid benzylester (diastereomer-1, 236 mg, 0.59 mmol) in DMF (1.5 mL) and methanol (48 μL, 1.19 mmol) was added a solution of lithium tert-butoxide (1.0 M solution in THF, 1.8 mL, 1.8 mmol) at room temperature. The solution was then cooled to 0° C. and (1S)-2-(acetylamino)-1-(chloromethyl)ethyl acetate (229 mg, 1.18 mmol) was added. The reaction was warmed up to room temperature and stirred overnight. Saturated aqueous ammonium chloride (2 mL) was added followed by water (20 mL). The reaction mixture was extracted with dichloromethane, washed with brine, and the organic layer was dried over MgSO₄. Solvent was removed and the residue was purified by preparative TLC (10% methanol/dichloromethane) to give the product as a white solid (153 mg, 64%). HPLC: retention time 4.23 minutes, purity >95%; [M+H]⁺ 407.7.

Diastereomer-2 (350 mg, 0.88 mmol) was carried out in a similar manner and the product was obtained as a white solid (273 mg, 77%). HPLC: retention time 4.22 minutes, purity >95%; [M+H]⁺ 407.7.
N-{3-[4-(3,4-Dihydroxy-cyclohexyl)-3-fluoro-Iphenyl]-2-oxo-oxazolidin-5-ylmethyl}-acetamide

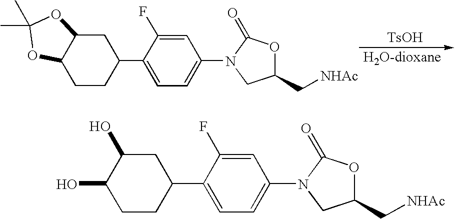

N-{3-[4-(2,2-Dimethyl-hexahydro-benzo[1,3]dioxol-5-yl)-3-fluoro-phenyl]-2-oxo-oxazolidin-5-ylmethyl}-acetamide (diastereomer-1, 153 mg, 0.38 mmol) was dissolved in 2 mL of H₂O-dioxane (1:4), and then catalytic amount of p-toluenesulfonic acid monohydrate (PTSA) was added to the solution. The reaction mixture was stirred overnight at room temperature, condensed, and purified by preparative TLC (10% methanol/dichloromethane) to give the desired product as a white solid (124 mg, 90%). ¹H NMR (300 MHz, DMSO) δ 8.23 (t, J=6.3 Hz, 1H), 7.42 (dd, J=12.9 Hz, 2.1 Hz, 1H), 7.32-7.20 (m, 2H), 4.70 (m, 1H), 4.50 (d, J=6.0 Hz, 1H), 4.24 (d, J=3.0 Hz, 1H), 4.09 (t, J=9.0 Hz, 1H), 3.77-3.68 (m, 2H), 2.78 (t, J=12.6 Hz, 1H), 1.82-1.32 (m, 6H); HPLC: retention time 3.82 minutes, purity >95%; [M+H]⁺ 367.6.

Diastereomer-2 (273 mg, 0.67 mmol) was carried out in a similar manner and the product was obtained as a white solid (220 mg, 89%). ¹H NMR (300 MHz, DMSO) δ 8.23 (t, J=5.7 Hz, 1H), 7.39 (dd, J=12.9 Hz, 2.4 Hz, 1H), 7.33-7.18 (m, 2H), 4.70 (m, 1H), 4.44 (d, J=6.0 Hz, 1H), 4.33 (d, J=2.7 Hz, 1H), 4.08 (t, J=9.0 Hz, 1H), 3.82-3.67 (m, 2H), 3.14 (m, 1H), 1.82-1.42 (m, 6H); HPLC: retention time 3.68 minutes, purity >95%; [M+H]⁺ 367.6.

### Example 2

### Preparation of N-{3-[3-Fluoro-4-(3-hydroxy-4-oxo-cyclohexyl)-phenyl]-2-oxo-oxazolidin-5-yl}-acetamide

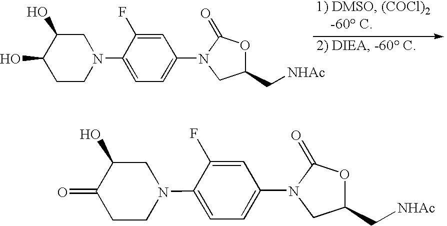

To a 25 mL flask were added 5.0 mL of dichloromethane and 105 μL (1.48 mmol) of DMSO. The mixture was then cooled to −65° C. and 64 μL (0.73 mmol) of oxalyl chloride was added dropwise. After 10 minutes, 226 mg (0.62 mmol) of N-{3-[4-(3,4-dihydroxy-piperidin-1-yl)-3-fluoro-phenyl]-2-oxo-oxazolidin-5-ylmethyl}-acetamide dissolved in 1.0 mL of dichloromethane was added to the reaction mixture at −65° C. The reaction mixture was then stirred for 50 minutes at −65° C., and 536 μL (3.08 mmol) of diisopropylethyl amine (DIEA) was added and stirred for another 10 minutes at −65° C. The reaction was then warmed up slowly to room temperature, quenched with saturated ammonium chloride solution, and extracted with dichloromethane. The organic layers were combined and washed with brine, dried over MgSO₄. Solvent was removed and the residue was purified by column chromatography (10% methanol/dichloromethane) to give the desired product as a white solid (180 mg, 80%). ¹H NMR (300 MHz, DMSO) δ 8.26 (t, J=5.7 Hz, 1H), 7.50 (d, J=14.1 Hz, 1H), 7.16-7.10 (m, 2H), 5.39 (d, J=5.4 Hz, 1H), 4.70 (m, 1H), 4.30 (m, 1H), 4.144.04 (m, 2H), 3.72-3.64 (m, 2H), 3.58-3.52 (m, 1H), 3.40-3.37 (m, 2H), 3.15 (d, J=5.4 Hz, 1H), 3.00 (dt, J=12.0 Hz, 2.7 Hz, 1H), 2.79 (t, J=11.1 Hz, 2H), 2.33 (d, J=14.1 Hz, 1H), 1.82 (s, 3H); HPLC: R.T. 3.14 min (broad peak), purity >90%; [M+H]⁺ 366.5.

### Example 3

### Preparation of N-{3-[3-Fluoro-4-(3-hydroxy-4-hydroxyimino-cyclohexyl)-phenyl]-2-oxo-oxazolidin-5-yl}-acetamide

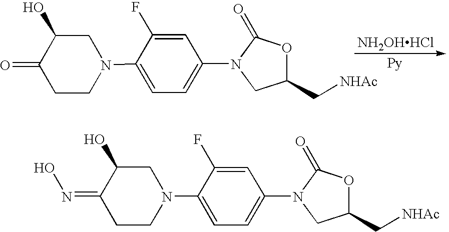

N-{3-[3-Fluoro-4-(3-hydroxy-4-oxo-piperidin-1-yl)-phenyl]-2-oxo-oxazolidin-5-ylmethyl}-acetamide (40 mg, 0.11 mmol) was dissolved in 1 mL of pyridine, and then hydroxylamine hydrochloride (12 mg, 0.17 mmol) was added. The reaction mixture was stirred for 1 hour at room temperature. Solvent was removed under high vacuum and the residue was purified by preparative TLC (10% methanol/dichloromethane) to give the desired oxime as a white solid (30 mg, 72%). ¹H NMR (300 MHz, DMSO) δ 10.67 (s, 1H), 8.23 (t, J=6.0 Hz, 1H), 7.46 (d, J=15.0 Hz, 1H), 7.16-7.03 (m, 2H), 5.10 (d, J=4.5 Hz, 1H), 4.69 (m, 1H), 4.15-4.04 (m, 2H), 3.71-3.65 (dd, J=9.0 Hz, 6.6 Hz, 1H), 3.38 (t, J=5.4 Hz, 2H), 3.25-3.07 (m, 3H), 2.89-2.59 (m, 3H), 1.82 (s, 3H); HPLC: R.T. 3.23 minutes, purity >95%; [M+H]⁺ 381.5.

### Example 4

### Preparation of N-{3-[3-Fluoro-4-(3-hydroxy-4-methoxyimino-cyclohexyl)-phenyl]-2-oxo-oxazolidin-5-yl}-acetamide

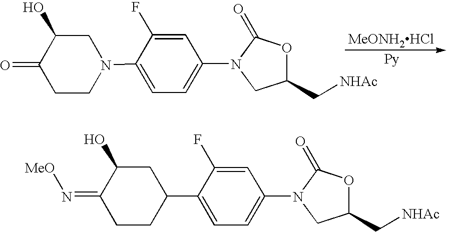

N-{3-[3-Fluoro-4-(3-hydroxy-4-oxo-piperidin-1-yl)-phenyl]-2-oxo-oxazolidin-5-ylmethyl}-acetamide (50 mg, 0.14 mmol) was dissolved in 1 mL of pyridine, and then methoxylamine hydrochloride (15 mg, 0.18 mmol) was added. The reaction mixture was stirred for 1 hour at room temperature. Solvent was removed under high vacuum and the residue was purified by preparative TLC (10% methanol/dichloromethane) to give the desired oxime as a white solid (31 mg, 57%). ¹H NMR (300 MHz, CDCl₃) δ 7.42 (dd, J=13.5 Hz, 2.4 Hz, 1H), 7.05 (dd, J=9.0 Hz, 1.8 Hz, 1H), 6.94 (t, J=9.0 Hz, 1H), 6.26 (m, 1H), 4.77 (m, 1H), 4.37 (dd, J=8.1 Hz, 4.5 Hz, 1H), 4.02 (t, J=9.0 Hz, 1H), 3.88 (s, 3H), 3.76-3.48 (m, 4H), 3.25-3.20 (m, 1H), 3.04-2.92 (m, 3H), 2.04 (s, 3H); HPLC: R.T. 3.67 minutes, purity >95%; [M+H]⁺ 395.5.

### Example 5

### Preparation of 3-[4-(3,4-Dihydroxy-cyclohexyl)-3-fluoro-phenyl]-5-[1,2,3]triazol-1-ylmethyl-oxazolidin-2-one

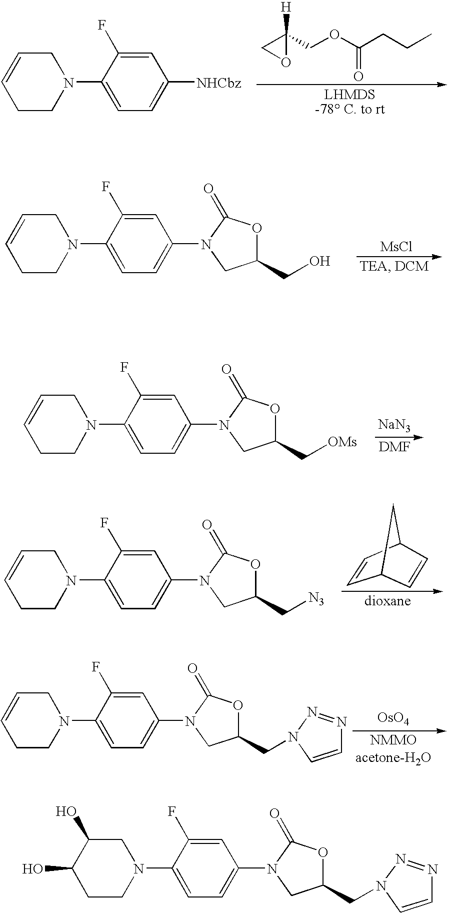 3-[4-(3,6-Dihydro-2H-pyridin-1-yl)-3-fluoro-phenyl]-5-hydroxymethyl-oxazolidin-2-one

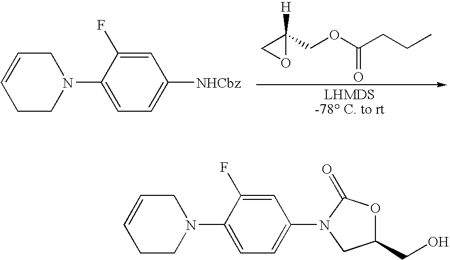

To a solution of [4-(3,6-dihydro-2H-pyridin-1-yl)-3-fluoro-phenyl]-carbamic acid benzyl ester (1.54 g, 4.72 mmol) in 20 mL of THF was added 5.2 mL of lithium hexamethyldisilazide (LHMDS) (1.0 M in THF, 5.19 mmol) at −78° C. After 30 minutes, 0.8 mL (5.66 mmol) of R-(−)-glycidylbutyrate was added and the reaction mixture was warmed up to room temperature and stirred overnight. The reaction was then quenched with saturated NH₄Cl solution, extracted with dichloromethane, and dried over MgSO₄. Solvent was removed and the residue was purified by column chromatography (10% methanol/dichloromethane) to give 1.21 g (87%) of the desired product as a white solid. HPLC: retention time 3.33 minutes, Purity >99%; [M+H]⁺ 293.3.
3-[4-(3,6-Dihydro-2H-pyridin-1-yl)-3-fluoro-phenyl]-5-[1,2,3]triazol-1-ylmethyl-oxazolidin-2-one

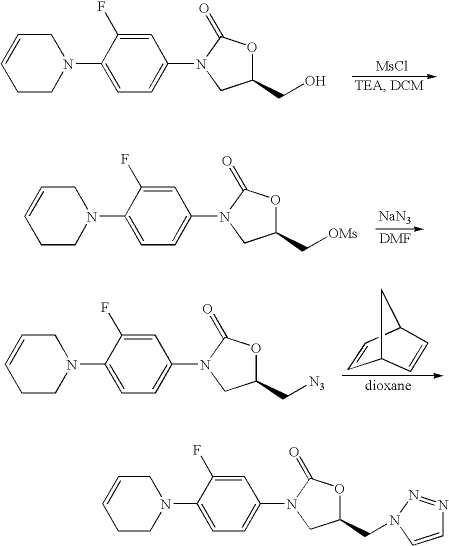

To a mixture of 3-[4-(3,6-dihydro-2H-pyridin-1-yl)-3-fluoro-phenyl]-5-hydroxymethyl-oxazolidin-2-one (1.0 g, 3.42 mmol) and DCM (15 mL) was added triethylamine (0.7 mL, 5.13 mmol) at 0° C. Methanesulfonyl chloride (292 μL, 3.76 mmol) was then added dropwise and the reaction mixture was warmed up gradually to room temperature and stirred for 1.5 hours. The reaction mixture was diluted with water, extracted with dichloromethane, and washed with diluted NaHCO₃ solution and brine. Solvent was removed and the reaction mixture (1.26 g) was used directly for the next step without further purification.

The mesylate (1.26 g) was taken up in 5 mL of DMF, treated with 1.0 g (15.38 mmol) of sodium azide, and heated at 50-60° C. overnight. The reaction mixture was then diluted with water, extracted with dichloromethane, and dried over MgSO₄. After removing the solvent, the residue was used directly for the next step.

The azide (100 mg, 0.31 mmol) was dissolved in 5 mL of dioxane, then 170 μL (1.55 mmol) of bicyclo [2.2.1.] hepta-2,5-diene was added and the reaction mixture was heated at 90-100° C. for 4 hours. Solvent was removed and the residue was purified by preparative TLC (10% methanol/EtOAc) to give the desired product (65 mg, 60%) as a brown solid. HPLC: retention time 3.78 minutes, Purity >99%; [M+H]⁺ 344.3.
3-[4-(3,4-Dihydroxy-piperidin-1-yl)-3-fluoro-phenyl]-5-[1,2,3]triazol-1-ylmethyl-oxazolidin-2-one

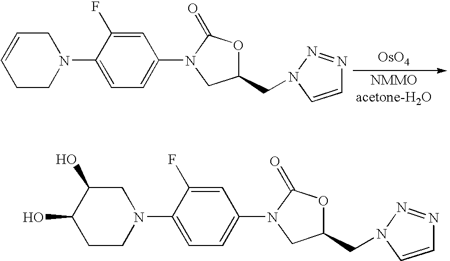

3-[4-(3,6-Dihydro-2H-pyridin-1-yl)-3-fluoro-phenyl]-5-[1,2,3]triazol-1-ylmethyl-oxazolidin-2-one (60 mg, 0.17 mmol) and 4-methylmorpholine N-oxide (NMMO, 45 mg, 0.37 mmol) were dissolved in 1 mL of acetone-H₂O (4:1). Then catalytic amount of osmium tetroxide (2.5 wt. % solution in 2-methyl-2-propanol) was added and the reaction mixture was stirred overnight at room temperature. The reaction mixture was then condensed and the residue was purified by preparative TLC (10% methanol/EtOAc) to give the product as a white solid (50 mg, 76%). ¹H NMR (300 MHz, DMSO) δ 8.16 (s, 1H), 7.76 (s, 1H), 7.36 (dd, J=14.7 Hz, 2.1 Hz, 1H), 7.10-6.99 (m, 2H), 5.01 (m, 1H), 4.81 (d, J=5.1 Hz, 2H), 4.60 (d, J=5.7 Hz, 1H), 4.45 (d, J=3.6 Hz, 1H), 4.18 (t, J=9.3 Hz, 1H), 3.83 (dd, J=9.3 Hz, 5.7 Hz, 1H), 3.75 (m, 1H), 3.63 (m, 1H), 3.40-3.33 (m, 2H), 2.99-2.79 (m, 4H), 1.82-1.64 (m, 2H); HPLC: retention time 3.30 minutes, purity >95%; [M+Na]⁺ 400.4.

### Example 6

### Preparation of 1-[2-Fluoro-4-(2-oxo-5-[1,2,3]triazol-1-ylmethyl-oxazolidin-3-yl)-phenyl]-3-hydroxy-piperidin-4-one

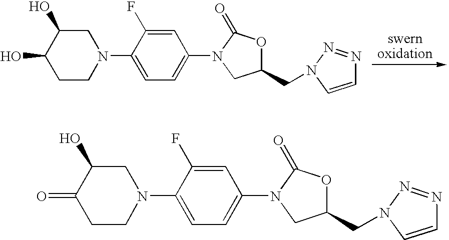 1-[2-Fluoro-4-(2-oxo-5-[1,2,3]triazol-1-ylmethyl-oxazolidin-3-yl)-phenyl]-3-hydroxy-piperidin-4-one

To a mixture of DMSO (38 μL, 0.53 mmol) in 1 mL of dichloromethane was added 24 μL (0.26 mmol) of oxalyl chloride at −60° C. After 15 minutes, a solution of 84 mg (0.22 mmol) of 3-[4-(3,4-dihydroxy-piperidin-1-yl)-3-fluoro-phenyl]-5-[1,2,3]triazol-1-ylmethyl-oxazolidin-2-one dissolved in 1 mL of dichloromethane and 1 mL of 1-methyl-2-pyrrolidinone was added dropwise to this mixture. The reaction mixture was stirred for 30 minutes at −60° C., then 194 μL of DIEA (1.10 mmol) was added and the reaction was warmed up to room temperature. Saturated ammonium chloride solution (1 mL) was added and the reaction mixture was extracted with dichloromethane, washed with brine, and dried over MgSO₄. Solvent was removed and the residue was purified by preparative TLC (10% methanol/dichloromethane) to give the product as a white solid (30 mg, 36%). ¹H NMR (300 MHz, DMSO) δ 8.16 (s, 1H), 7.76 (s, 1H), 7.42 (d, J=13.8 Hz, 1H), 7.12 (m, 2H), 5.11 (m, 1H), 4.81 (d, J=5.1 Hz, 2H), 4.30 (dd, J=9.9 Hz, 6.6 Hz, 1H), 4.20 (t, J=9.3 Hz, 1H), 3.85 (dd, J=9.3 Hz, 5.7 Hz, 1H), 3.70-3.52 (m, 2H), 3.00 (dt, J=11.7 Hz, 3.0 Hz, 1H), 2.82-2.68 (m, 3H), 2.33 (d, J=14.1 Hz, 1H); HPLC: R.T. 3.76 minutes, purity >95%; [M+Na]⁺ 376.4.

### Example 8

### Preparation of N-{3-[4-(3,4-Dihydroxy-cyclohexyl)-3-fluoro-phenyl]-2-oxo-oxazolidin-5-ylmethyl}-propionamide

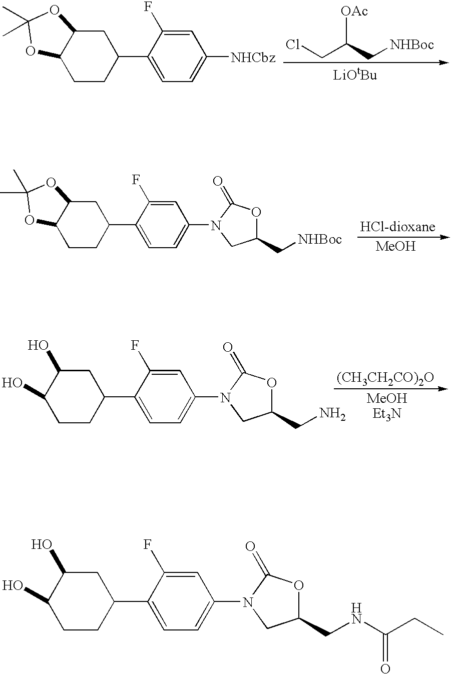 {3-[4-(2,2-Dimethyl-hexahydro-benzo[1,3]dioxol-5-yl)-3-fluoro-phenyl]-2-oxo-oxazolidin-5-ylmethyl}-carbamic acid tert-butyl ester

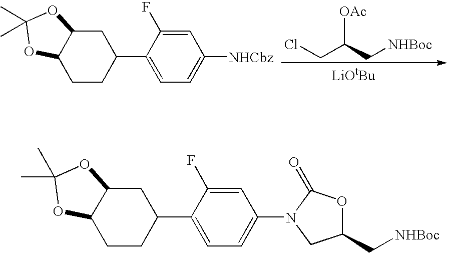

To a solution of [4-(2,2-dimethyl-hexahydro-benzo[1,3]dioxol-5-yl)-3-fluoro-phenyl]-carbamic acid benzylester (940 mg, 2.36 mmol) and acetic acid 1(S)-(tert-butoxycarbonylamino-methyl)-2-chloro-ethyl ester (622 mg, 1.26 mmol) was added a solution of lithium t-butoxide (1.0 M solution in ThF, 5.65 mL, 5.65 mmol) at 0° C. The reaction mixture was then warmed up to room temperature and stirred for 40 hours. Saturated aqueous ammonium chloride (10 mL) was added followed by water (40 mL). The reaction mixture was extracted with dichloromethane, washed with brine, and the organic layer was dried over MgSO₄. Solvent was removed and the residue was purified by preparative TLC (75% EtOAc/hexanes) to give the product as a viscous oil (820 mg, 75%). HPLC: retention time 5.03 minutes, purity >95%; [M+Na]⁺ 487.8.
N-{3-[4-(3,4-Dihydroxy-cyclohexyl)-3-fluoro-phenyl]-2-oxo-oxazolidin-5-ylmethyl}-propionamide

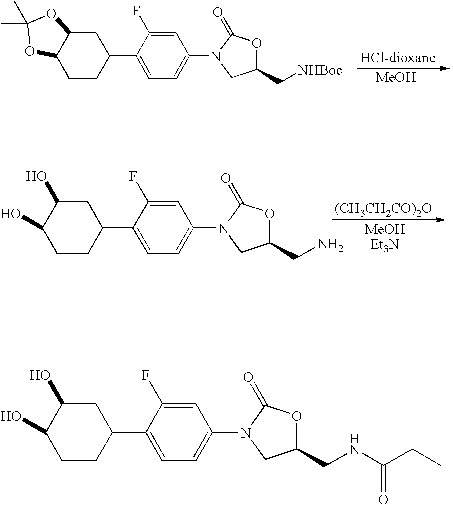

To a solution of {3-[4-(2,2-dimethyl-hexahydro-benzo[1,3]dioxol-5-yl)-3-fluoro-phenyl)-2-oxo-oxazolidin-5-ylmethyl}-carbamic acid tert-butyl ester (53 mg, 0.11 mmol) in 1.0 mL of methanol was added 0.3 mL of HCl solution (4.0 M in dioxane). The reaction mixture was then stirred for two hours at room temperature. Solvent was removed under vacuum and the residue was dried under high vacuum.

The free amine was dissolved in 2 mL of methanol and 64 μL of triethylamine (0.46 mmol) followed by 29 μL of propionic anhydide (0.23 mmol) were added. The reaction was stirred for one hour at room temperature, condensed, and purified by preparative TLC (10% methanol/dichloromethane) to give the product as a white solid (30 mg, 69%). ¹H NMR (300 MHz, DMSO) δ 8.16 (t, J=6.0 Hz, 1H), 7.38 (dd, J=13.2 Hz, 2.1 Hz, 1H), 7.33-7.18 (m, 2H), 4.70 (m, 1H), 4.08 (t, J=9.0 Hz, 1H), 2.07 (q, J=7.8 Hz, 2H), 0.94 (t, J=7.2 Hz, 3H); HPLC: retention time 3.73 minutes, purity >95%; [M+H]⁺ 381.3.

### Example 9

### Preparation of 2,2-Dichloro-N-{3-[4-(3,4-dihydroxy-cyclohexyl)-3-fluoro-phenyl]-2-oxo-oxazolidin-5-ylmethyl}-acetamide

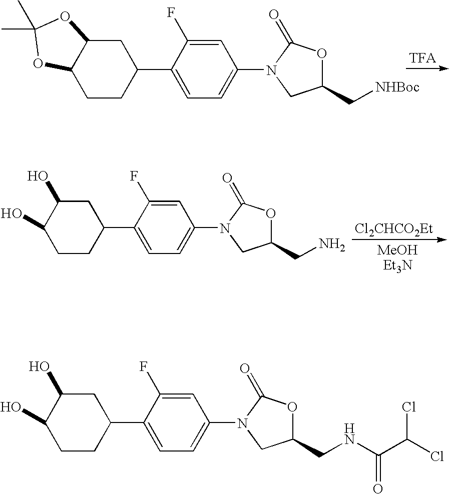 2,2-Dichloro-N-{3-[4-(3,4-dihydroxy-cyclohexyl)-3-fluoro-phenyl]-2-oxo-oxazolidin-5-ylmethyl}-acetamide

To a solution of {3-[4-(2,2-dimethyl-hexahydro-benzo[1,3]dioxol-5-yl)-3-fluoro-phenyl]-2-oxo-oxazolidin-5-ylmethyl}-carbamic acid tert-butyl ester (74 mg, 0.16 mmol) in 1,2-dichloroethane (DCE, 2 mL) was added 0.5 mL of trifluoroacetic acid (TFA). The reaction mixture was then stirred for two hours at room temperature. Solvent was removed under vacuum and the residue was dried under high vacuum.

The free amine was dissolved in 2 mL of methanol and 67 μL of triethylamine (0.48 mmol) followed by 79 μL of ethyl dichloroacetate (0.64 mmol) were added. The reaction was stirred overnight at room temperature, condensed, and purified by preparative TLC (80% EtOAc/hexanes) to give the product as a white solid (35 mg, 50%). ¹H NMR (300 MHz, DMSO) δ 8.99 (t, J=5.7 Hz, 1H), 7.41-7.17 (m, 3H), 4.78 (m, 1H), 4.43 (d, J=6.0 Hz, 1H), 4.32 (d, J=2.7 Hz, 1H), 4.12 (t, J=9.0 Hz, 1H), 3.82-3.69 (m, 2H), 3.14 (t, J=12.0 Hz, 1H), 1.77-1.38 (m, 4H); HPLC: retention time 4.08 minutes, purity >95%; [M+Na]⁺ 458.1.

### Example 10

### Preparation of N-{3-[4-(3,4-Dihydroxy-cyclohexyl)-3-fluoro-phenyl]-2-oxo-oxazolidin-5-ylmethyl}-2,2-difluoro-acetamide

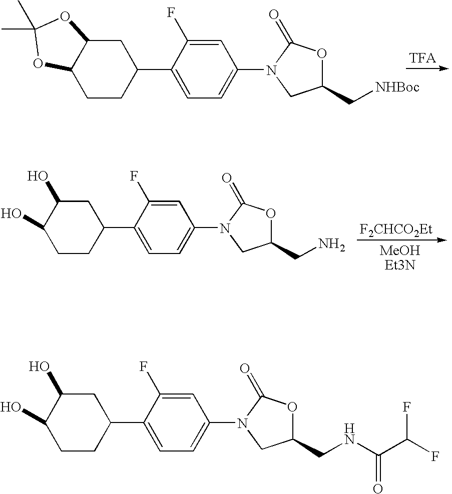

To a solution of {3-[4-(2,2-dimethyl-hexahydro-benzo[1,3]dioxol-5-yl)-3-fluoro-phenyl]-2-oxo-oxazolidin-5-ylmethyl}-carbamic acid tert-butyl ester (150 mg, 0.32 mmol) in 1,2-dichloroethane (DCE, 4 mL) was added 1 mL of trifluoroacetic acid (TFA). The reaction mixture was then stirred for two hours at room temperature. Solvent was removed under vacuum and the residue was dried under high vacuum.

The deprotection product was dissolved in 3 mL of methanol and 90 μL of triethylamine (0.64 mmol) followed by 300 μL of ethyl difluoroacetate were added. The reaction was stirred for three hours at room temperature, condensed, and purified by preparative TLC (80% EtOAc/hexanes) to give the product as a white solid (70 mg, 54%). ¹H NMR (300 MHz, DMSO) δ 9.15 (t, J=5.7 Hz, 1H), 7.41-7.18 (m, 3H), 6.24 (t, J=53.7 Hz, 1H), 4.78 (m, 1H), 4.43 (d, J=6.0 Hz, 1H), 4.32 (d, J=2.7 Hz, 1H), 4.12 (t, J=9.0 Hz, 1H), 3.82-3.72 (m, 2H), 3.51 (t, J=5.4 Hz, 1H), 1.77-1.38 (m, 4H); HPLC: R.T. 3.78 minutes, purity >95%; [M+H]⁺ 403.3.

### Example 11

### Preparation of N-{3-[4-(3,4-Dihydroxy-cyclohexyl)-3-fluoro-phenyl]-2-oxo-oxazolidin-5-ylmethyl}-2,2-difluoro-thioacetamide

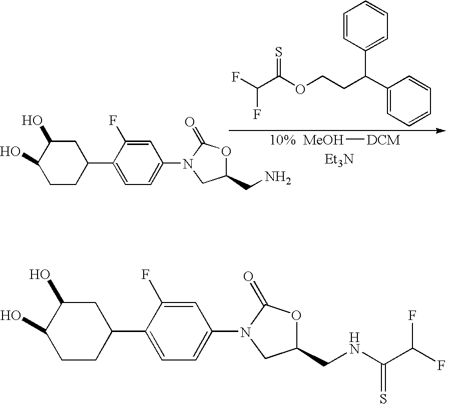

To a solution of N-{3-[4-(3,4-dihydroxy-cyclohexyl)-3-fluoro-phenyl]-2-oxo-oxazolidin-5-ylmethyl}-amine (51 mg, 0.16 mmol) in 2 mL of MeOH-dichloromethane (1:9) was added difluoro-thioacetic acid O-(3,3-diphenyl-propyl) ester (58 mg, 0.19 mmol) followed by triethylamine (46 μL, 0.32 mmol). The reaction was stirred overnight at room temperature, condensed, and purified by preparative TLC (10% methanol/dichloromethane) to give the product as a white solid (55 mg, 83%). ¹H NMR (300 MHz, DMSO) δ 7.42-7.19 (m, 3H), 6.48 (t, J=55.2 Hz, 1H), 5.00 (m, 1H), 4.44 (d, J=6.0 Hz, 1H), 4.33 (d, J=2.7 Hz, 1H), 4.16 (t, J=9.3 Hz, 1H), 3.96-3.81 (m, 3H), 1.77-1.42 (m, 4H); HPLC: retention time 4.50 minutes, purity >95%; [M+H]⁺ 419.2.

### Example 12

### Preparation of N-{3-[4-(3,4-Dihydroxy-cyclohexyl)-phenyl]-2-oxo-oxazolidin-5-ylmethyl}-acetamide

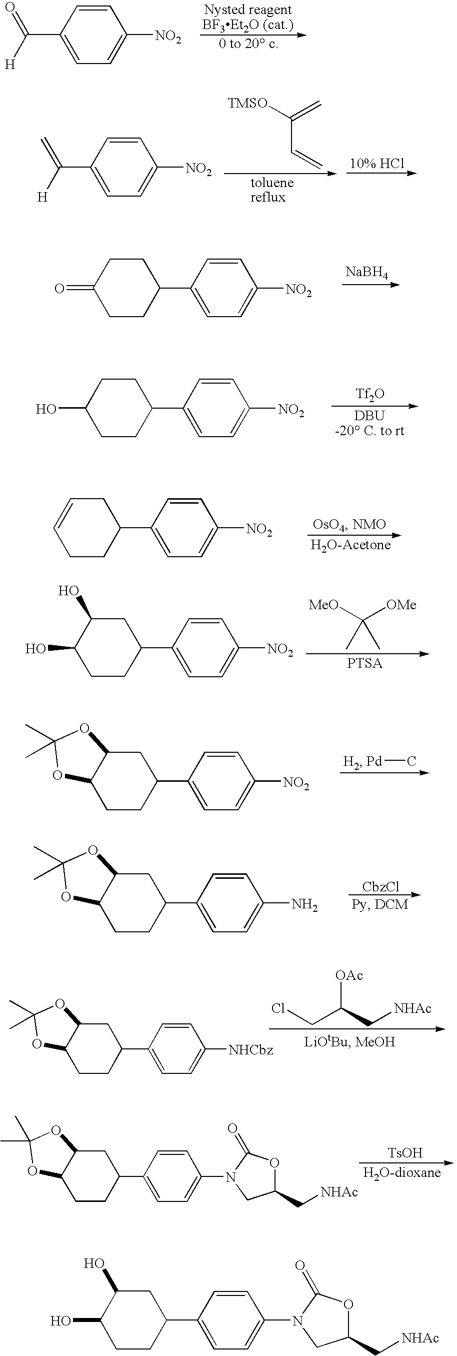 4-(4-Nitro-phenyl)-cyclohexanone

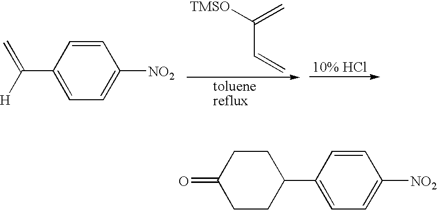

4-Nitrostyrene (1.0 g, 6.7 mmol) and 2-trimethylsilyloxy-1,3-butadiene (3.5 mL, 20.1 mmol) were mixed with 15 mL of anhydrous toluene. The mixture was placed in a sealed tube and reflused for 48 hours. The reaction mixture was then cooled to room temperature and poured into 10% HCl. The solution was then stirred for 1 hour at room temperature and extracted with EtOAc. The combined organic layers were washed with diluted NaHCO₃, brine and dried over MgSO₄. The solvent was removed and the residue was purified by column chromatography (15% EtOAc/hexanes). The product was obtained as a pale yellow solid (600 mg, 41%). HPLC: retention time 4.33 minutes, purity >95%.
1-Cyclohex-3-enyl-4-nitro-benzene

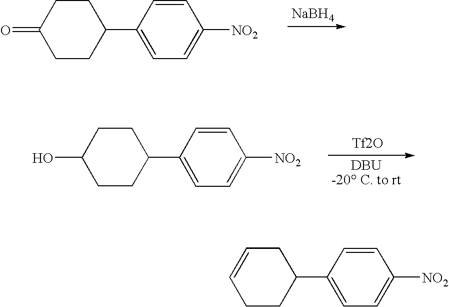

To a solution of 4-(4-nitro-phenyl)-cyclohexanone (600 mg, 2.74 mmol) in 10 mL of absolute ethanol was added 207 mg (5.48 mmol) of sodium borohydride. The solution was then stirred for 2 hours at room temperature, and quenched carefully with 10% HCl solution. The reaction mixture was then extracted with EtOAc, and the combined organic layers were washed with brine and dried over MgSO₄. Solvent was removed under vacuum and the alcohol (590 mg, 97%) was used directly for the next step without further purification.

The alcohol (590 mg, 2.67 mmol) was dissolved in 10 mL of dichloromethane, and the solution was cooled to −20° C. DBU (974/L, 6.50 mmol) was then added followed by trifluoromethanesulfonic anhydride (548 μL, 3.25 mmol). The reaction mixture was then warmed up to room temperature and quenched with water, extracted with EtOAc, and the organic layer was washed with brine. The solvent was removed under vacuum, and the residue was purified by column chromatography (30% EtOAc/hexanes) to give the desired product (487 mg, 90%) as a yellow oil.
4-(4-Nitro-phenyl)-cyclohexane-1,2-diol

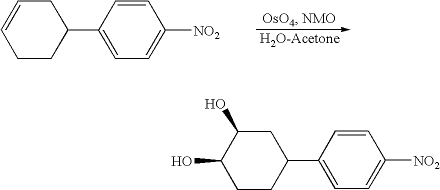

1-Cyclohex-3-enyl-4-nitro-benzene (1.70 g, 8.37 mmol) was dissolved in 20 mL of acetone-water (4:1) solution. Then NMMO (1.96 g, 16.74 mmol) was added followed by osmium tetroxide (2.5 wt. % solution in Bu^{t}OH, 320 μL). The reaction was stirred overnight, concentrated, and purified by preparative TLC (20% EtOAc/hexanes). The product was collected as a white solid (1.80 g, 91%). HPLC: retention time 3.72 min (mixture of two diastereomers), purity >95%.
2,2-Dimethyl-5-(4-nitro-phenyl)-hexahydro-benzo[1,3]dioxole

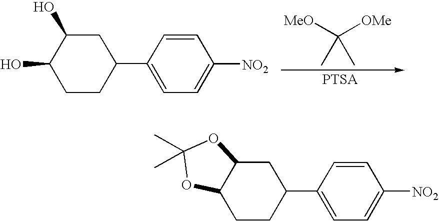

To a solution of 4-(4-nitro-phenyl)-cyclohexane-1,2-diol (400 mg, 1.69 mmol) in 2.0 mL of 2,2-dimethoxypropane was added catalytic amount of p-toluenesulfonic acid monohydrate (PTSA). The reaction was then stirred at room temperature for 2 hours. Solvent was removed under vacuum and the residue was purified by preparative TLC (15% EtOAc/hexanes). The TLC plates were developed twice and the two diastereomers were separated very carefully. Both the upper spot (diastereomer-1, 270 mg) and the lower spot (diastereomer-2, 160 mg) were obtained as pale yellow solids.
Diastereomer-1: HPLC: retention time 4.93 minutes, purity >95%.
Diastereomer-2: HPLC: retention time 4.93 minutes, purity >95%.
   4-(2,2-Dimethyl-hexahydro-benzo[1,3]dioxol-5-yl)-phenylamine

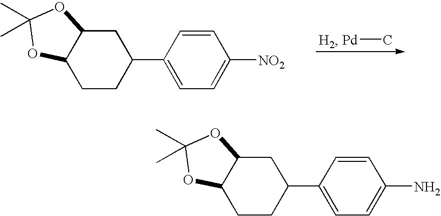

2,2-Dimethyl-5-(4-nitro-phenyl)-hexahydro-benzo[1,3]dioxole (diastereomer-1, 560 mg, 2.02 mmol) was dissolved in 15 mL of MeOH, and 50 mg of Pd—C (5 wt. % on activated carbon) catalyst was added to the solution. A balloon filled with hydrogen was placed on top of the flask and the reaction mixture was stirred overnight at room temperature. The solution was then passed through a short celite pad and washed with methanol. The collected solution was condensed under vacuum and the residue was dried under high vacuum. The desired product was obtained as a viscous liquid (488 mg, 98%). HPLC: retention time 3.01 minutes, purity >95%; [M+H]⁺ 248.6.

Diastereomer-2 (330 mg, 1.19 mmol) was carried out in a similar manner and the product was obtained as a glassy solid (247 mg, 84%). HPLC: retention time 3.01 minutes, purity >95%; [M+H]⁺ 248.6.
[4-(2,2-Dimethyl-hexahydro-benzo[1,3]dioxol-5-yl)-Iphenyl]-carbamic acid benzyl ester

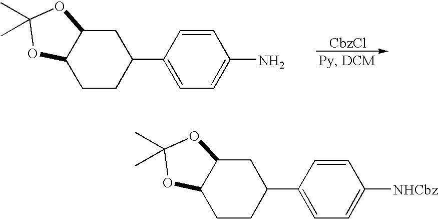

Benzyl chloroformate (338 μL, 2.36 mmol) was added dropwise to a mixture of 4-(2,2-dimethyl-hexahydro-benzo[1,3]dioxol-5-yl)-phenylamine (diastereomer-1, 488 mg, 1.97 mmol) and pyridine (383 μL, 4.74 mmol) in dichloromethane (15 mL) at 0° C. The reaction mixture was stirred for 30 minutes at 0° C., and then warmed up to room temperature. The reaction mixture was poured into water, extracted with EtOAc, and the organic layer was separated, washed with brine and dried over MgSO₄. The solvent was then removed and the residue was purified by preparative TLC (25% EtOAc/hexanes). The desired product was obtained as a foaming solid (645 mg, 86%). HPLC: retention time 5.33 minutes, purity >95%; [M+Na]⁺ 404.6.

Diastereomer-2 (296 mg, 1.20 mmol) was carried out in a similar manner and the product was obtained as a foaming solid (350 mg, 77%). HPLC: retention time 5.30 minutes, purity >95%; [M+Na]⁺ 404.6.
N-{3-[4-(2,2-Dimethyl-hexahydro-benzo[1,3]dioxol-5-yl)-phenyl]-2-oxo-oxazolidin-5-ylmethyl}-acetamide

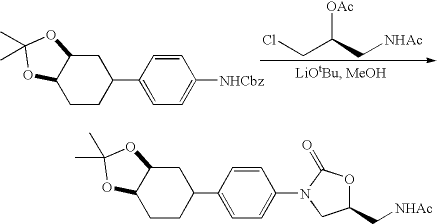

To a solution of [4-(2,2-dimethyl-hexahydro-benzo[1,3]dioxol-5-yl)-phenyl]-carbamic acid benzylester (diastereomer-1, 300 mg, 0.79 mmol) in DMF (1.5 mL) and methanol (64 μL, 1.59 mmol) was added a solution of lithium tert-butoxide (1.0 M solution in THF, 2.36 mL, 2.36 mmol) at room temperature. The solution was then cooled to 0° C. and (1S)-2-(acetylamino)-1-(chloromethyl)ethyl acetate (305 mg, 1.57 mmol) was added. The reaction was warmed up to room temperature and stirred overnight. Saturated aqueous ammonium chloride (2 mL) was added followed by water (20 mL). The reaction mixture was extracted with dichloromethane, washed with brine, and the organic layer was dried over MgSO₄. Solvent was removed and the residue was purified by preparative TLC (10% methanol/dichloromethane) to give the product as a white solid (270 mg, 88%). HPLC: retention time 4.58 minutes, purity >95%; [M+H]⁺ 389.3.

Diastereomer-2 (300 mg, 0.79 mmol) was carried out in a similar manner and the product was obtained as a white solid (273 mg, 89%). HPLC: retention time 4.55 minutes, purity >95%; [M+H]⁺ 389.3.
N-{3-[4-(3,4-Dihydroxy-cyclohexyl)-phenyl]-2-oxo-oxazolidin-5-ylmethyl}-acetamide

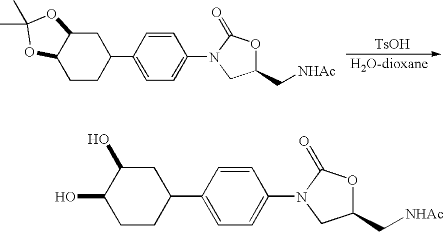

N-{3-[4-(2,2-Dimethyl-hexahydro-benzo[1,3]dioxol-5-yl)-phenyl]-2-oxo oxazolidin-5-ylmethyl}-acetamide (diastereomer-1, 90 mg, 0.23 mmol) was dissolved in 2 mL of H₂O-dioxane (1:4), and then catalytic amount of p-toluenesulfonic acid monohydrate (PTSA) was added to the solution. The reaction mixture was stirred overnight at room temperature, condensed, and purified by preparative TLC (10% methanol/dichloromethane) to give the desired product as a white solid (55 mg, 68%). ¹H NMR (300 MHz, DMSO) δ 8.23 (t, J=5.7 Hz, 1H), 7.40 (d, J=8.4 Hz, 2H), 7.21 (d, J=8.7 Hz, 2H), 4.68 (m, 1H), 4.39 (d, J=6.0 Hz, 1H), 4.28 (d, J=2.7 Hz, 1H), 4.07 (t, J=9.0 Hz, 1H), 3.81-3.67 (m, 2H), 2.84 (tt, J=12.0 Hz, 3.0 Hz, 1H), 1.82-1.34 (m, 6H); HPLC: retention time 3.52 minutes, purity >95%; [M+H]⁺ 349.2.

Diastereomer-2 (90 mg, 0.23 mmol) was carried out in a similar manner and the product was obtained as a white solid (50 mg, 62%). ¹H NMR (300 MHz, DMSO) 88.23 (t, J=5.7 Hz, 1H), 7.42 (d, J=8.7 Hz, 2H), 7.20 (d, J=8.7 Hz, 2H), 4.68 (m, 1H), 4.43 (d, J=5.7 Hz, 1H), 4.19 (d, J=2.7 Hz, 1H), 4.07 (t, J=9.0 Hz, 1H), 3.76-3.68 (m, 2H), 1.82-1.34 (m, 6H); HPLC: retention time 3.60 minutes, purity >95%; [M+H]⁺ 349.3.

### Example 13

### Preparation of 2,2-Dichloro-N-{3-[4-(3,4-dihydroxy-cyclohexyl)-phenyl]-2-oxo-oxazolidin-5-ylmethyl}-acetamide

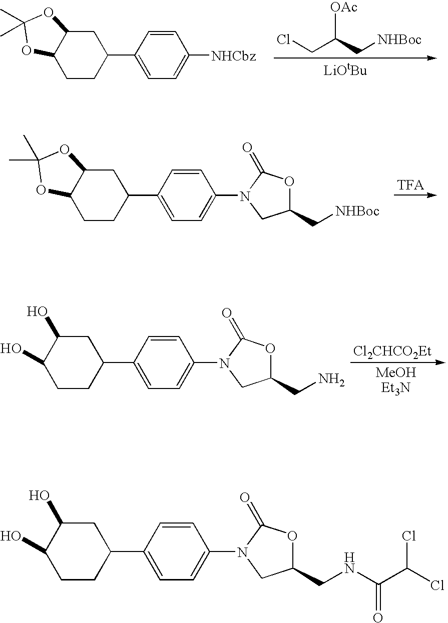 {3-[4-(2,2-Dimethyl-hexahydro-benzo[1,3]dioxol-5-yl)-phenyl]-2-oxo-oxazolidin-5-ylmethyl}-carbamic acid tert-butyl ester

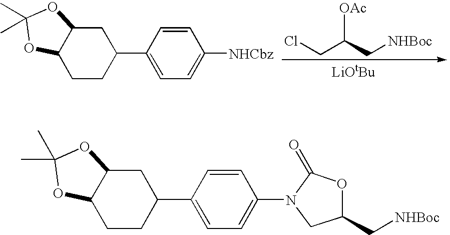

To a solution of [4-(2,2-dimethyl-hexahydro-benzo[1,3]dioxol-5-yl)-phenyl]-carbamic acid benzylester (326 mg, 0.86 mmol) and acetic acid 1(S)-(tert-butoxycarbonylamino-methyl)-2-chloro-ethyl ester (226 mg, 1.08 mmol) was added a solution of lithium t-butoxide (1.0 M solution in THF, 2.10 mL, 2.10 mmol) at 0° C. The reaction mixture was then warmed up to room temperature and stirred for 40 hours. Saturated aqueous ammonium chloride (10 mL) was added followed by water (40 mL). The reaction mixture was extracted with dichloromethane, washed with brine, and the organic layer was dried over MgSO₄. Solvent was removed and the residue was purified by preparative TLC (75% EtOAc/hexanes) to give the product as a viscous oil (300 mg, 79%). HPLC: retention time 5.45 minutes, purity >95%; [M+H]⁺ 447.3.
2,2-Dichloro-N-{3-[4-(3,4-dihydroxy-cyclohexyl)-phenyl]-2-oxo-oxazolidin-5-ylmethyl}-acetamide

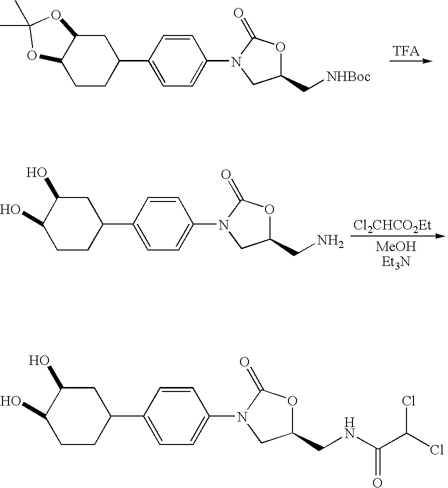

To a solution of {3-[4-(2,2-dimethyl-hexahydro-benzo[1,3]dioxol-5-yl)-phenyl]-2-oxo-oxazolidin-5-ylmethyl}-carbamic acid tert-butyl ester (60 mg, 0.13 mmol) and 1,2-dichloroethane (DCE, 2.0 mL) was added 0.5 mL of trifluoroacetic acid (TFA). The reaction mixture was then stirred for two hours at room temperature. Solvent was removed and the residue was dried under high vacuum.

The free amine was dissolved in 2 mL of methanol and 56 μL of triethylamine (0.40 mmol) followed by 66 mL of ethyl dichloroacetate (0.54 mmol) were added. The reaction was stirred overnight at room temperature, condensed, and purified by preparative TLC (80% EtOAc/hexanes) to give the product as a white solid (25 mg, 45%). ¹H NMR (300 MHz, DMSO) δ 7.40 (d, J=8.4 Hz, 2H), 7.21 (d, J=8.7 Hz, 2H), 4.76 (m, 1H), 4.11 (t, J=9.0 Hz, 1H), 3.81-3.69 (m, 2H), 2.83 (tt, J=12.3 Hz, 3.0 Hz, 1H), 1.80-1.37 (m, 4H); HPLC: retention time 3.97 minutes, purity >95%; [M+Na]⁺ 440.2.

### Example 14

### Preparation of N-{3-[4-(3,4-Dihydroxy-cyclohexyl)-phenyl]-2-oxo-oxazolidin-5-ylmethyl}-2,2-difluoro-acetamide

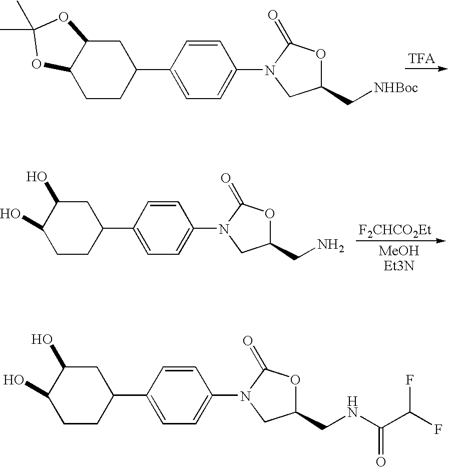

To a solution of {3-[4-(2,2-dimethyl-hexahydro-benzo[1,3]dioxol-5-yl)-phenyl]-2-oxo-oxazolidin-5-ylmethyl}-carbamic acid tert-butyl ester (60 mg, 0.13 mmol) and 1,2-dichloroethane (DCE, 4.0 mL) was added 1 mL of trifluoroacetic acid (TFA). The reaction mixture was then stirred for two hours at room temperature. Solvent was removed under vacuum and the residue was dried under high vacuum.

The free amine was dissolved in 3 mL of methanol and 37 μL of triethylamine (0.26 mmol) and 300 mL of ethyl difluoroacetate were added. The reaction was stirred for three hours at room temperature, condensed, and purified by preparative TLC (80% EtOAc/hexanes) to give the product as a white solid (30 mg, 59%). ¹H NMR (300 MHz, DMSO) δ 9.20 (t, J=5.7 Hz, 1H), 7.40 (d, J=9.0 Hz, 2H), 7.21 (d, J=9.0 Hz, 2H), 6.25 (t, J=53.7 Hz, 1H), 4.76 (m, 1H), 4.42 (d, J=6.3 Hz, 1H), 4.30 (d, J=2.7 Hz, 1H), 4.11 (t, J=9.0 Hz, 1H), 3.81-3.73 (m, 2H), 3.53-3.41 (m, 3H), 2.84 (tt, J=12.3 Hz, 3.3 Hz, 1H), 1.80-1.34 (m, 4H); HPLC: retention time 3.72 minutes, purity >95%; [M+Na]⁺ 407.3.

### Example 15

### Preparation of N-{3-[4-(3,4-Dihydroxy-cyclohexyl)-phenyl]-2-oxo-oxazolidin-5-ylmethyl}-2,2-difluoro-thioacetamide

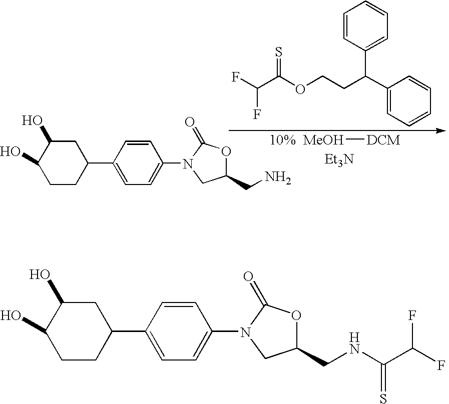

To a solution of N-{3-[4-(3,4-dihydroxy-cyclohexyl)-3-fluoro-phenyl]-2-oxo-oxazolidin-5-ylmethyl}-amine (69 mg, 0.22 mmol) in 2 mL of MeOH-dichloromethane (1:9) was added difluoro-thioacetic acid Q-(3,3-diphenyl-propyl) ester (82 mg, 0.27 mmol) followed by triethylamine (63 μL, 0.44 mmol). The reaction was stirred overnight at room temperature, condensed, and purified by preparative TLC (10% methanol/dichloromethane) to give the product as a white solid (50 mg, 81%). ¹H NMR (300 MHz, DMSO) δ 7.41 (d, J=9.0 Hz, 2H), 7.21 (d, J=8.7 Hz, 2H), 6.49 (t, J=55.2 Hz, 1H), 4.98 (m, 1H), 4.41 (d, J=6.0 Hz, 1H), 4.29 (d, J=2.7 Hz, 1H), 4.15 (t, J=9.0 Hz, 1H), 3.96-3.80 (m, 3H), 2.84 (tt, J=12.3 Hz, 3.3 Hz, 1H), 1.80-1.34 (m, 4H); HPLC: retention time 4.38 minutes, purity >95%; [M+H]⁺ 401.2.

### Example 16

### Preparation of N-{3-[3-Fluoro-4-(4-hydroxy-cyclohexyl)-phenyl]-2-oxo-oxazolidin-5-ylmethyl}-acetamide

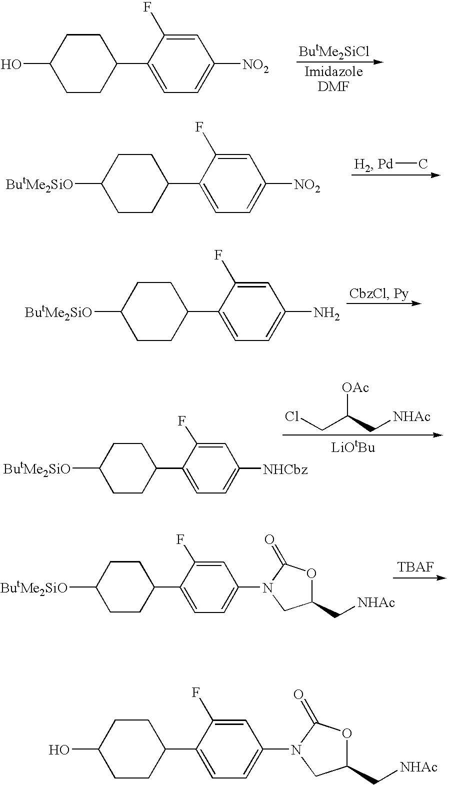 tert-Butyl-[4-(2-fluoro-4-nitro-phenyl)-cyclohexvloxy]-dimethyl-silane

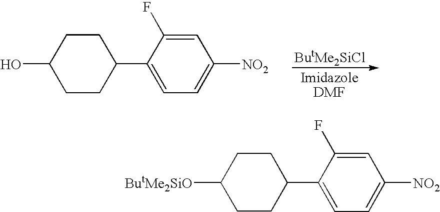

To a solution of 4-(2-fluoro-4-nitro-phenyl)-cyclohexanol (500 mg, 2.09 mmol) in 3 mL of DMF was added imidazole (427 mg, 6.27 mmol), followed by tert-butyldimethylsilyl chloride (TBS, 473 mg, 3.13 mmol) at 0° C. The reaction mixture was then warmed up to room temperature and stirred for another hour. The reaction was quenched with water, extracted with ethyl acetate, and the organic layer was washed with brine and dried over MgSO₄. Solvent was removed to give the desired product as a viscous liquid (700 mg, 95%). HPLC: retention time 7.47 minutes, purity >95%.
4-[4-(tert-Butyl-dimethyl-silanyloxy)-cyclohexyl]-3-fluoro-phenylamine

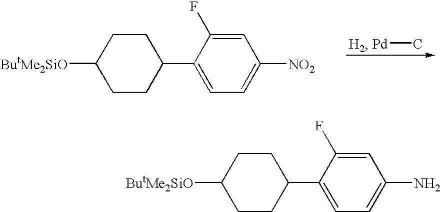

tert-Butyl-[4-(2-fluoro-4-nitro-phenyl)-cyclohexyloxy]-dimethyl-silane (700 mg, 1.98 mmol) was dissolved in 15 mL of mrthanol, and 50 mg of Pd-C (5 wt. % on activated carbon) catalyst was added to the solution. A balloon filled with hydrogen was placed on top of the flask and the reaction mixture was stirred overnight at room temperature. The solution was then passed through a short celite pad and washed with methanol. The collected solution was condensed under vacuum and the residue was dried under high vacuum. The desired product was obtained as a viscous liquid (600 mg, 94%). HPLC: retention time 5.82 minutes, purity >95%; [M+H]⁺ 324.3.
{4-[4-(tert-Butyl-dimethyl-silanyloxy)-cyclohexyl]-3-fluoro-phenyl}-carbamic acid benzyl ester

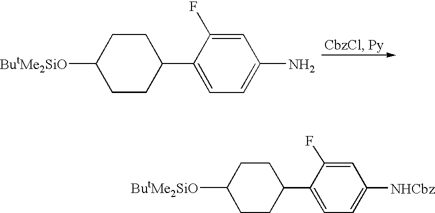

Benzyl chloroformate (345 μL, 2.41 mmol) was added dropwise to a mixture of 4-[4-(tert-butyl-dimethyl-silanyloxy)-cyclohexyl]-3-fluoro-phenylamine (650 mg, 2.01 mmol) and pyridine (391 μL, 4.82 mmol) in dichloromethane (15 mL) at 0° C. The reaction mixture was stirred for 30 minutes at 0° C., and then warmed up to room temperature. The reaction mixture was poured into water, extracted with EtOAc, and the organic layer was separated, washed with brine and dried over MgSO₄. The solvent was then removed and the residue was purified by preparative TLC (25% EtOAc/hexanes). The desired product was obtained as a foaming solid (900 mg, 98%). HPLC: retention time 7.75 minutes, purity >95%; [M+Na]⁺ 480.3.
N-(3-{4-[4-(tert-Butyl-dimethyl-silanyloxy)-cyclohexyl]-3-fluoro-phenyl}-2-oxo-oxazolidin-5-ylmethyl)-acetamide

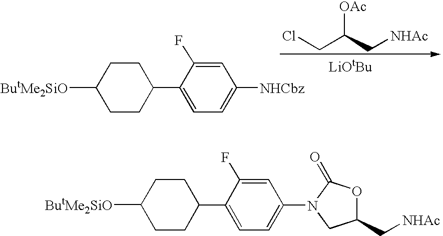

To a solution of {4-[4-(tert-butyl-dimethyl-silanyloxy)-cyclohexyl]-3-fluoro-phenyl}-carbamic acid benzyl ester (524 mg, 1.15 mmol) in DMF (1.5 mL) and methanol (94 μL, 2.32 mmol) was added a solution of lithium tert-butoxide (1.0 M solution in THF, 3.4 mL, 3.40 mmol) at room temperature. The solution was then cooled to 0° C. and (1S)-2-(acetylamino)-1-(chloromethyl)ethyl acetate (444 mg, 2.30 mmol) was added. The reaction was warmed up to room temperature and stirred overnight. Saturated aqueous ammonium chloride (2 mL) was added followed by water (20 mL). The reaction mixture was extracted with dichloromethane, washed with brine, and the organic layer was dried over MgSO₄. Solvent was removed and the residue was purified by preparative TLC (80% EtOAc/hexanes) to give the product as a white solid (346 mg, 65%). HPLC: retentnion time 6.67 minutes, purity >95%; [M+H]⁺ 465.3.
N-{3-[3-Fluoro-4-(4-hydroxy-cyclohexyl)-phenyl]-2-oxo-oxazolidin-5-ylmethyl}-acetamide

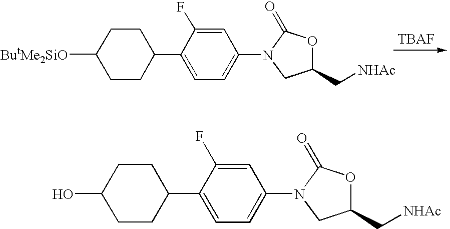

N-(3-{4-[4-(tert-Butyl-dimethyl-silanyloxy)-cyclohexyl]-3-fluoro-phenyl}-2-oxo-oxazolidin-5-ylmethyl)-acetamide (61 mg, 0.13 mmol) was dissolved in 1 mL of THF. Then tetrabutylammonium fluoride (TBAF, 1.0 M solution in THF, 0.26 mL, 0.26 mmol) was added and the reaction mixture was stirred overnight at room temperature. The reaction was quenched with water, extracted with ethyl acetate and the organic layers were combined and dried over MgSO₄. Solvent was removed and the residue was further purified by preparative TLC (10% methanol/dichloromethane) to give the desired product as a white solid (35 mg, 76%). ¹H NMR (300 MHz, DMSO) δ 8.23 (t, J=5.1 Hz, 1H), 7.40 (dd, J=13.2 Hz, 2.1 Hz, 2H), 7.33-7.18 (m, 2H), 4.69 (m, 1H), 4.59 (d, J=4.5 Hz, 1H), 4.08 (t, J=9.0 Hz, 1H), 3.69 (dd, J=9.0 Hz, 6.6 Hz, 1H), 2.67 (t, J=12.3 Hz, 1H), 1.92-1.21 (m, 5H); HPLC: retention time 4.90 minutes, purity >95%; [M+H]⁺ 351.2.

### Example 17

### Preparation of

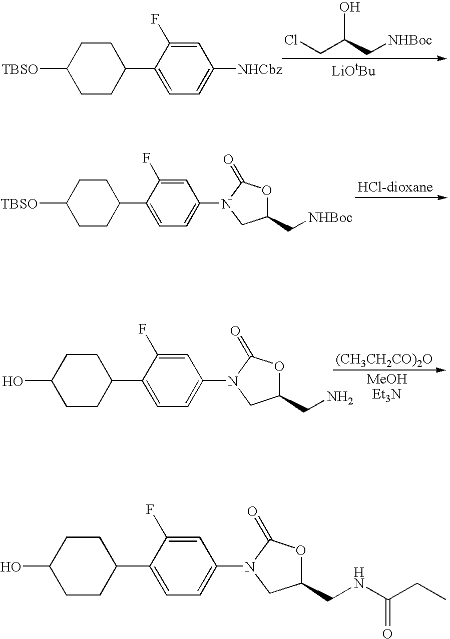 (3-{4-[4-(tert-Butyl-dimethyl-silanyloxy)-cyclohexyl]-3-fluoro-phenyl}-2-oxo-oxazolidin-5-ylmethyl)-carbamic acid tert-butyl ester

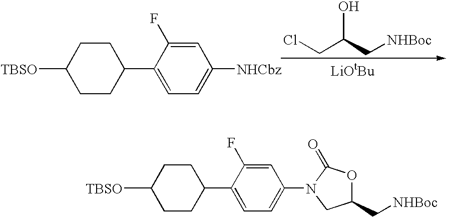

To a solution of {4-[4-(tert-butyl-dimethyl-silanyloxy)-cyclohexyl]-3-fluoro-phenyl}-carbamic acid benzyl ester (548 mg, 1.20 mmol) and acetic acid 1(S)-(tert-butoxycarbonylamino-methyl)-2-chloro-ethyl ester (317 mg, 1.51 mmol) was added a solution of lithium t-butoxide (1.0 M solution in THF, 2.9 mL, 2.90 mmol) at 0° C. The reaction mixture was then warmed up to room temperature and stirred for 40 hours. Saturated aqueous ammonium chloride (10 mL) was added followed by water (40 mL). The reaction mixture was extracted with dichloromethane, washed with brine, and the organic layer was dried over MgSO₄. Solvent was removed and the residue was purified by preparative TLC (75% EtOAc/hexanes) to give the product as a viscous oil (510 mg, 81%). HPLC: retention time 7.18 minutes, purity >95%.
N-{3-[3-Fluoro-4-(4-hydroxy-cyclohexyl)-phenyl]-2-oxo-oxazolidin-5-ylmethyl}-propionamide

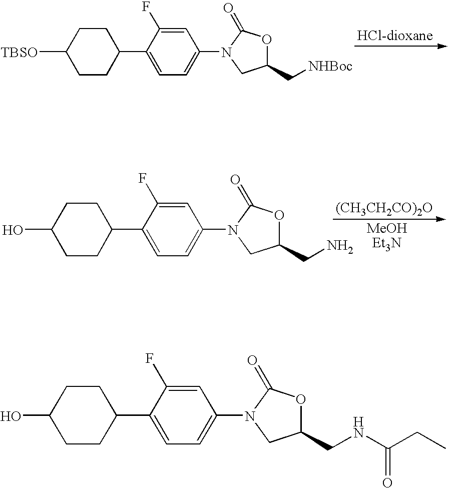

To a solution of (3-{4-[4-(tert-butyl-dimethyl-silanyloxy)-cyclohexyl]-3-fluoro-phenyl}-2-oxo-oxazolidin-5-ylmethyl)-carbamic acid tert-butyl ester (150 mg, 0.29 mmol) in 3 mL of methanol was added 1 mL of HCl solution (4.0 M in dioxane). The reaction mixture was then stirred for two hours at room temperature. Solvent was removed and the residue was dried under high vacuum.

The free amine was dissolved in 2 mL of methanol and 160 μL of triethylamine (1.16 mmol) followed by 74 μL of propionic anhydide (0.58 mmol) were added. The reaction was stirred for one hour at room temperature, condensed, and purified by preparative TLC (10% methanol/dichloromethane) to give the product as a white solid (80 mg, 76%). ¹H NMR (300 MHz, DMSO) δ 8.15 (t, J=5.7 Hz, 1H), 7.40 (dd, J=13.2 Hz, 2.1 Hz, 2H), 7.33-7.18 (m, 2H), 4.70 (m, 1H), 4.59 (d, J=4.5 Hz, 1H), 4.08 (t, J=9.6 Hz, 1H), 3.70 (dd, J=9.0 Hz, 6.3 Hz, 1H), 2.67 (t, J=12.0 Hz, 1H), 2.07 (q, J=7.8 Hz, 2H), 1.92-1.25 (m, 8H), 0.93 (t, J=7.5 Hz, 3H); HPLC: retention time 4.88 minutes, purity >95%; [M+Na]⁺ 387.2.

### Example 18

### Preparation of 2,2-Difluoro-N-{3-[3-fluoro-4-(4-hydroxy-cyclohexyl)-phenyl]-2-oxo-oxazolidin-5-ylmethyl}-acetamide

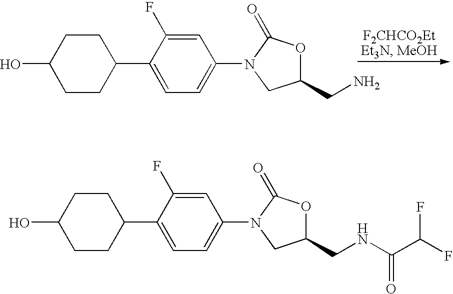 5-Aminomethyl-3-[3-fluoro-4-(4-hydroxy-cyclohexyl)-phenyl]-oxazolidin-2-one (32 mg, 0.10 mmol) was dissolved in 1 mL of methanol and 29 μL of triethylamine (0.20 mmol) and 200 μL of ethyl difluoroacetate were added. The reaction was stirred for three hours at room temperature, condensed, and purified by preparative TLC (80% EtOAc/hexanes) to give the product as a white solid (31 mg, 77%). ¹H NMR (300 MHz, DMSO) δ 7.42-7.18 (m, 3H), 6.24 (t, J=53.4 Hz, 1H), 4.77 (m, 1H), 4.58 (d, J=4.5 Hz, 1H), 4.12 (t, J=9.0 Hz, 1H), 3.74 (dd, J=9.3 Hz, 6.3 Hz, 1H), 2.68 (t, J=11.7 Hz, 1H), 1.92-1.21 (m, 6H); HPLC: retention time 4.18 minutes, purity >95%; [M+H]⁺ 387.2.

### Example 19

### 2,2-Difluoro-N-{3-[3-fluoro-4-(4-hydroxy-cyclohexyl)-phenyl]-2-oxo-oxazolidin-5-ylmethyl}-thioacetamide

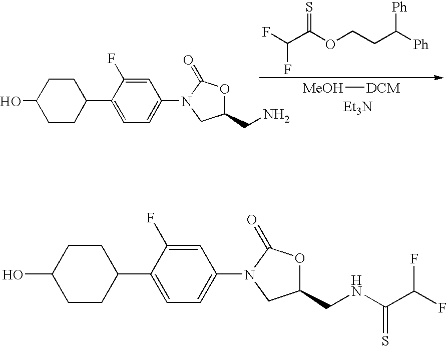

To a solution of 5-aminomethyl-3-[3-fluoro-4-(4-hydroxy-cyclohexyl)-phenyl]-oxazolidin-2-one (61 mg, 0.20 mmol) in 2 mL of MeOH-dichloromethane (1:9) was added difluoro-thioacetic acid O-(3,3-diphenyl-propyl) ester (122 mg, 0.40 mmol) followed by triethylamine (56 mL, 0.40 mmol). The reaction was stirred overnight at room temperature, condensed, and purified by preparative TLC (10% methanol/dichloromethane) to give the product as a white solid (68 mg, 85%). ¹H NMR (300 MHz, DMSO) δ 7.43-7.19 (m, 3H), 6.48 (t, J=55.2 Hz, 1H), 5.00 (m, 1H), 4.58 (d, J=4.2 Hz, 1H), 4.16 (t, J=9.0 Hz, 1H), 3.96-3.80 (m, 3H), 2.68 (t, J₁=11.7 Hz, 1H), 1.92-1.21 (m, 8H); HPLC: retention time 4.77 minutes, purity >95%; [M+H]⁺ 403.3.

### Example 20

### Preparation of N-{3-[3-Fluoro-4-(4-oxo-cyclohexyl)-phenyl]-2-oxo-oxazolidin-5-ylmethyl}-acetamide

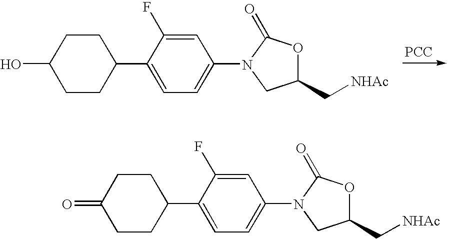

N-{3-[3-Fluoro-4-(4-hydroxy-cyclohexyl)-phenyl]-2-oxo-oxazolidin-5-ylmethyl}-acetamide (102 mg, 0.29 mmol) was dissolved in 5 mL of dichloromethane. Pyridinium chlorochromate (PCC, 126 mg, 0.58 mmol) was then added followed by celite (200 mg). The reaction mixture was stirred overnight at room temperature, passed through a short silica gel pad and washed with dichloromethane. Solvent was removed and the residue was purified by preparative TLC (10% methanol/dichloromethane) to give the desired product as a white solid (82 mg, 81%). ¹H NMR (300 MHz, DMSO) δ 8.23 (t, J=5.7 Hz, 1H), 7.48-7.21 (m, 3H), 4.70 (m, 1H), 4.09 (t, J=9.0 Hz, 1H), 3.70 (dd, J=9.3 Hz, 6.6 Hz, 1H), 2.60 (td, J=14.4 Hz, 6.3 Hz, 2H), 2.28-1.82 (m, 8H); HPLC: retention time 5.02 minutes, purity >95%; [M+H]⁺ 349.2.

### Example 21

### N-{3-[3-Fluoro-4-(4-hydroxyimino-cyclohexyl)-phenyl]-2-oxo-oxazolidin-5-ylmethyl}-acetamide

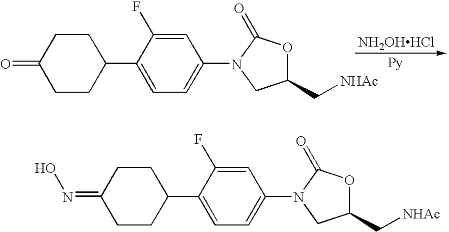

N-{3-[3-Fluoro-4-(4-oxo-cyclohexyl)-phenyl]-2-oxo-oxazolidin-5-ylmethyl}-acetamide (30 mg, 0.086 mmol) was dissolved in 1 mL of pyridine, and then hydroxylamine hydrochloride (12 mg, 0.17 mmol) was added. The reaction mixture was stirred for 1 hour at room temperature. Solvent was removed under high vacuum and the residue was purified by preparative TLC (10% methanol/dichloromethane) to give the desired oxime as a white solid (20 mg, 64%). ¹H NMR (300 MHz, DMSO) δ 8.23 (t, J=5.4 Hz, 1H), 7.46-7.19 (m, 3H), 4.70 (m, 1H), 4.09 (t, J=9.0 Hz, 1H), 3.70 (dd, J=9.3 Hz, 6.6 Hz, 1H), 3.03 (t, J=12.0 Hz, 1H), 2.37-2.17 (m, 2H), 1.91-1.44 (m, 7H); HPLC: retention time 4.07 minutes, purity >95%; [M+H]⁺ 364.2.

### Example 22

### N-{3-[3-Fluoro-4-(4-methoxyimino-cyclohexyl)-phenyl]-2-oxo-oxazolidin-5-ylmethyl}-acetamide

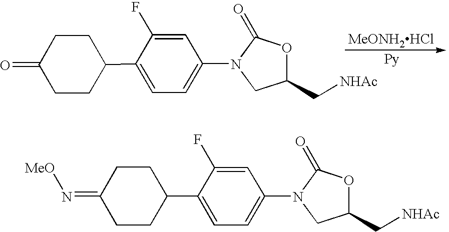

N-{3-[3-Fluoro-4-(4-oxo-cyclohexyl)-phenyl]-2-oxo-oxazolidin-5-ylmethyl}-acetamide (71 mg, 0.20 mmol) was dissolved in 1 mL of pyridine, and then methoxy]amine hydrochloride (34 mg, 0.40 mmol) was added. The reaction mixture was stirred for 1 hour at room temperature. Solvent was removed under high vacuum and the residue was purified by preparative TLC (10% methanol/dichloromethane) to give the desired oxime as a white solid (55 mg, 71%). ¹H NMR (300 MHz, DMSO) δ 8.23 (t, J=5.4 Hz, 1H), 7.46-7.19 (m, 3H), 4.70 (m, 1H), 4.08 (t, J=9.0 Hz, 1H), 3.72 (s, 3H), 3.04 (t, J=12.0 Hz, 1H), 2.37-2.20 (m, 2H), 1.93-1.46 (m, 7H); HPLC: R.T. 4.65 minutes, purity >95%; [M+H]⁺ 378.3.

### Example 23

### Preparation of 3-[3-Fluoro-4-(4-hydroxy-cyclohexyl)-phenyl]-2-oxo-oxazolidine-5-carboxylic acid amide

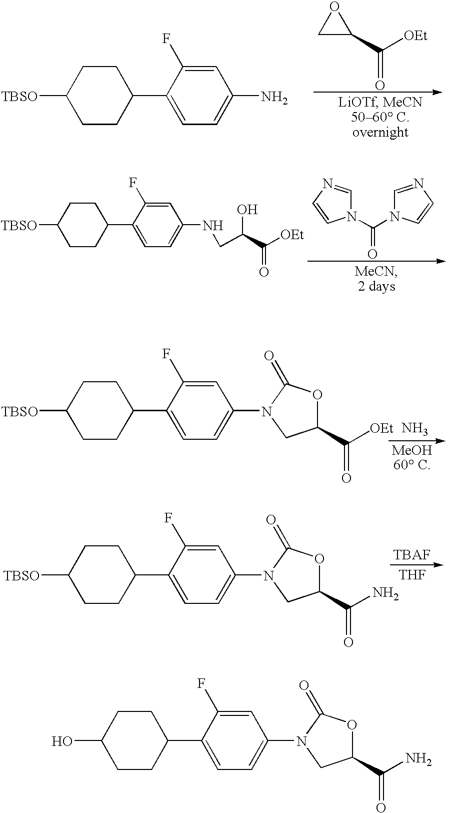 3-{4-[4-(tert-Butyl-dimethyl-silanyloxy)-cyclohexyl]-3-fluoro-phenylamino}-2-hydroxy-propionic acid ethyl ester

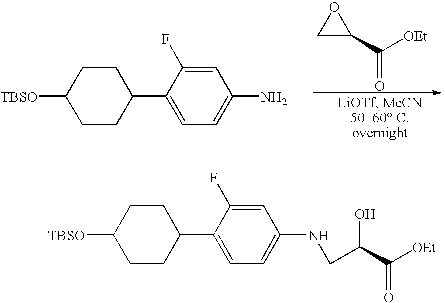

4-[4-(tert-Butyl-dimethyl-silanyloxy)-cyclohexyl]-3-fluoro-phenylamine (270 mg, 0.84 mmol) and (1S)-2-(acetylamino)-1-(chloromethyl)ethyl acetate (194 mg, 1.68 mmol) were dissolved in 5 mL of anhydrous acetonitrile, and then 196 mg (1.26 mmol) of lithium triflate was added. The reaction mixture was then heated at 50-60° C. overnight. Solvent was removed and the residue was purified by preparative TLC (30% EtOAc/hexanes) to give 200 mg (54%) of the desired product as a colorless oil. HPLC: retention time 7.88 minutes, purity >95%; [M+H]⁺ 440.3.
3-{4-[4-(tert-Butyl-dimethyl-silanyloxy)-cyclohexyl]-3-fluoro-phenyl}-2-oxo-oxazolidine-5-carboxylic acid amide

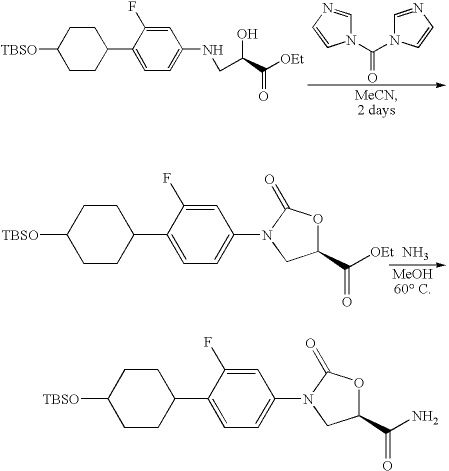

3-{4-[4-(tert-Butyl-dimethyl-silanyloxy)-cyclohexyl]-3-fluoro-phenylamino}-2-hydroxy-propionic acid ethyl ester (200 mg, 0.46 mmol) was dissolved in 4 mL of anhydrous acetonitrile and then 166 mg (1.01 mmol) of carbonyldiimidazole (CDI) was added. The reaction mixture was stirred for three days at room temperature. After the reaction was completed, solvent was removed and the residue was taken up in dichloromethane, washed with 3% citric acid solution, brine, and dried with MgSO₄. Solvent was removed and the residue was used directly for the next step without further purification.

3-{4-[4-(tert-Butyl-dimethyl-silanyloxy)-cyclohexyl]-3-fluoro-phenyl}-2-oxo-oxazolidine-5-carboxylic acid ethyl ester (200 mg, 0.43 mmol) was dissolved in 4 mL of ammonia (1.0 M in methanol) and the reaction mixture was heated at 60° C. for 1 hour. Solvent was removed and the residue was purified by preparative TLC (10% methanol/dichloromethane). The product (160 mg, 86%) was obtained as a colorless oil. HPLC R.T. 7.81 minutes, purity >99%; [M+H]⁺ 437.3.
3-[3-Fluoro-4-(4-hydroxy-cyclohexyl)-phenyl]-2-oxo-oxazolidine-5-carboxylic acid amide

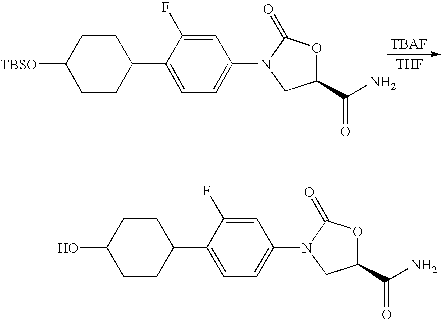

3-{4-[4-(tert-Butyl-dimethyl-silanyloxy)-cyclohexyl]-3-fluoro-phenyl}-2-oxo-oxazolidine-5-carboxylic acid amide (160 mg, 0.37 mmol) was dissolved in 1 mL of THF, and then 0.73 mL (1.0 M in THF, 0.74 mmol) of tetrabutylammonium fluoride (TBAF) was added. The reaction mixture was then stirred overnight at room temperature. After the reaction was completed, the mixture was taken up in dichloromethane, washed with brine and dried over MgSO₄. Solvent was removed and the residue was purified by preparative TLC (20% methanol/EtOAc). The product was obtained as a white solid (90 mg, 76%). ¹H NMR (300 MHz, DMSO)δ 7.85 (s, 1H), 7.61 (s, 1H), 7.45-7.25 (m, 3H), 5.00 (m, 1H), 4.58 (d, J=4.5 Hz, 1H), 4.24 (t, J=9.3 Hz, 1H), 3.97 (dd, J=9.3 Hz, 6.3 Hz, 1H), 3.43 (m, 1H), 2.68 (tt, J=12.0 Hz, 3.3 Hz, 1H), 1.92-1.21 (m, 8H); HPLC: retention time 4.45 minutes, purity >95%; [M+H]⁺ 323.4.

### Example 24

### 3-[3-Fluoro-4-(4-oxo-cyclohexyl)-phenyl]-2-oxo-oxazolidine-5-carboxylic acid amide

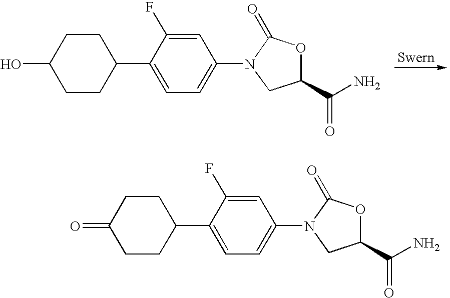

To a mixture of DMSO (66 μL, 0.96 mmol) in 1 mL of dichloromethane was added 41 μL (0.48 mmol) of oxalyl chloride at −60° C. After 15 minutes, a solution of 50 mg (0.16 mmol) of 3-[3-fluoro-4-(4-hydroxy-cyclohexyl)-phenyl]-2-oxo-oxazolidine-5-carboxylic acid amide dissolved in 1 mL of dichloromethane and 1 mL of DMSO was added dropwise to this mixture. The reaction mixture was stirred for 30 minutes at −60° C., then 162 μL of DIEA (0.96 mmol) was added and the reaction was warmed up to room temperature. Saturated ammonium chloride solution (1 mL) was added and the reaction mixture was extracted with dichloromethane, washed with brine, and dried over MgSO₄. Solvent was removed and the residue was purified by preparative TLC (20% methanol/EtOAc) to give the desired product as a white solid (25 mg, 50%). ¹H NMR (300 MHz, DMSO)δ 7.86 (s, 1H), 7.61 (s, 1H), 7.51-7.28 (m, 3H), 5.00 (dd, J=9.3 Hz, 5.7 Hz, 1H), 4.25 (t, J=9.3 Hz, 1H), 3.98 (dd, J=9.3 Hz, 6.0 Hz, 1H), 2.60 (m, 2H), 2.28-1.83 (m, 6H); HPLC: retention time 4.68 minutes, purity >95%; [M+H]⁺ 321.4.

### Example 25

### Preparation of N-{3-[3-Fluoro-4-(3-hydroxy-4-oxo-cyclohexyl)-phenyl]-2-oxo-oxazolidin-5-ylmethyl}-acetamide

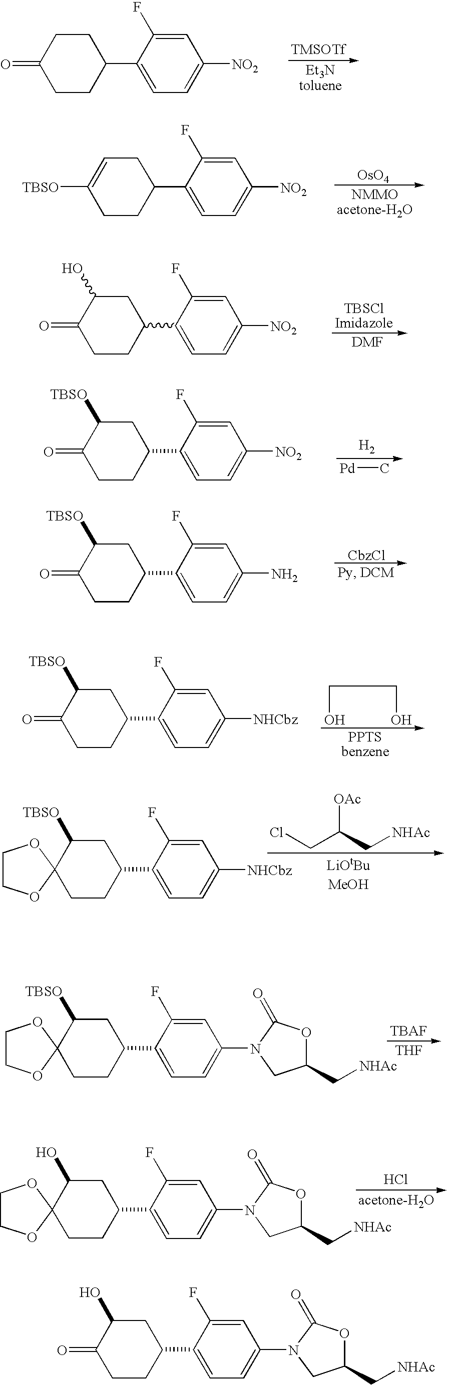 4-(2-Fluoro-4-nitro-phenyl)-2-hydroxy-cyclohexanone

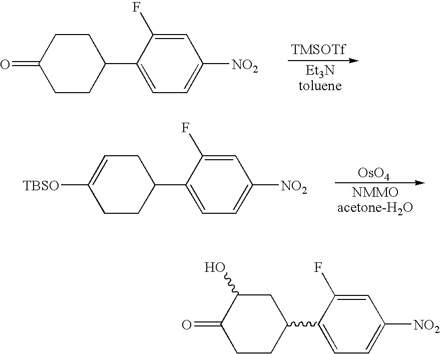

To a stirred solution of 4-(2-fluoro-4-nitro-phenyl)-cyclohexanone (200 mg, 0.84 mmol) in dry toluene (5 mL) was added 0.54 mL (3.88 mmol) of dry triethylamine followed by 0.46 mL (2.52 mmol) of trimethylsilyl trifluoromethanesulfonate. The reaction mixture was refluxed for 2 hours, during which time the solution turned from yellow to brown color, and some precipitate appeared. The reaction mixture was then washed with diluted NaHCO₃ solution, extracted with hexanes, and washed with brine and dried over MgSO₄. Solvent was then removed to give a yellow solid which was used directly for the next step.

The silyl enol ether (150 mg, 0.48 mmol) and 4-methylmorpholine N-oxide (NMMO, 125 mg, 1.05 mmol) were dissolved in 3.0 mL of acetone-H₂O (4:1). Then catalytic amount of osmium tetroxide (2.5 wt. % solution in 2-methyl-2-propanol) was added and the reaction mixture was stirred overnight at room temperature. The reaction mixture was then condensed and the residue was purified by preparative TLC (50% hexanes/EtOAc) to give the desired product as a white solid (86 mg, 70%). HPLC: retention time 5.00 min.
2-(tert-Butyl-dimethyl-silanyloxy)-4-(2-fluoro-4-nitro-phenyl)-cyclohexanone

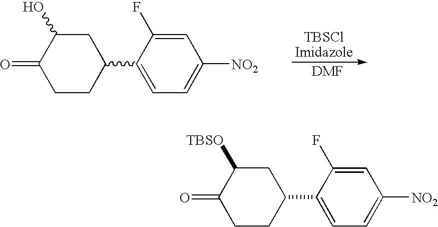

To a stirred mixture of 4-(2-fluoro-4-nitro-phenyl)-2-hydroxy-cyclohexanone (50 mg, 0.20 mmol) in 1 mL of DMF was added 40 mg (0.60 mmol) of imidazole followed by 45 mg (0.30 mmol) of tert-butyldimethylsilyl chloride (TBSCl) at 0° C. The reaction mixture was then warmed up gradually to room temperature, quenched with water, extracted with ethyl acetate and washed with brine. Solvent was removed and the residue was purified by preparative TLC (20% EtOAc/hexanes). Two isomers were isolated:
Upper spot: 15 mg, pale yellow solid
HPLC: retention time 7.66 minutes
Lower spot: 33 mg, pale yellow liquid (major isomer, used for the next few steps)
HPLC: retention time 7.62 minutes
   4-(4-Amino-2-fluoro-phenyl)-2-(tert-butyl-dimethyl-silanyloxy)-cyclohexanone

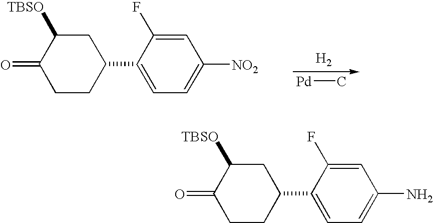

2-(tert-Butyl-dimethyl-silanyloxy)-4-(2-fluoro-4-nitro-phenyl)-cyclohexanone (125 mg, 0.34 mmol) was dissolved in 8 mL of methanol, and then 15 mg of Pd-C (10% on activated carbon) was added. The flask was then connected to vacuum and purged with hydrogen. The reaction mixture was stirred under hydrogen overnight, filtered through a short celite pad, and washed with methanol. Solvent was then removed to give the desired product as a pale yellow liquid (102 mg, 89%). HPLC: retention time 5.85 minutes, Purity >99%; [M+Na]⁺ 360.4.
{4-[3-(tert-Butyl-dimethyl-silanyloxy)-4-oxo-cyclohexyl]-3-fluoro-phenyl}-carbamic acid benzyl ester

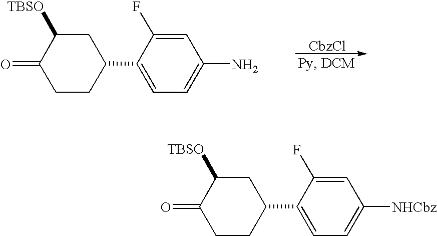

Benzyl chloroformate (52 μL, 0.36 mmol) was added dropwise to a mixture of 4-(4-Amino-2-fluoro-phenyl)-2-(tert-butyl-dimethyl-silanyloxy)-cyclohexanone (102 mg, 0.30 mmol) and pyridine (59 μL, 0.72 mmol) in dichloromethane (3 mL) at 0° C. The reaction mixture was warmed up to room temperature and stirred for 1 hour, then it was poured into water and extracted with ethyl acetate (20 mL×2). The organic layer was collected and washed with brine, and dried over MgSO₄. Solvent was removed and the residue was purified by preparative TLC (25% EtOAc/hexanes) to give the product as a white solid (110 mg, 77%). HPLC: retention time 7.93 minutes, Purity >99%; [M+H]⁺ 472.4.

{4-[6-(tert-Butyl-dimethyl-silanyloxy)-1,4-dioxa-spiro[4.5]dec-8-yl]-3-fluoro-phenyl}-carbamic acid benzyl ester

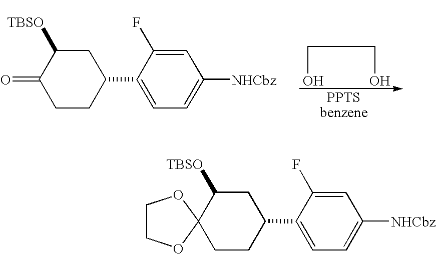

To a solution of {4-[3-(tert-butyl-dimethyl-silanyloxy)-4-oxo-cyclohexyl]-3-fluoro-phenyl}-carbamic acid benzyl ester (90 mg, 0.19 mmol) in benzene (15 mL) was added ethylene glycol (1 mL) followed by catalytic amount of pyridinium p-toluenesulfonate (PPTS). The flask was then attached to a dean-star trap and the reaction mixture was heated to 100° C. After refluxing overnight the reaction mixture was cooled to room temperature and poured into diluted NaHCO₃ solution, extracted with ethyl acetate, washed with brine and dried over MgSO₄. Solvent was removed and the residue (foaming solid, 90 mg, 92%) was used directly for the next step without further purification. HPLC: retention time 8.25 minutes, Purity >99%; [M+Na]⁺ 538.3.
N-(3-{4-[6-(tert-Butyl-dimethyl-silanyloxy)-1,4-dioxa-spiro[4.5]dec-8-yl]-3-fluoro-phenyl}-2-oxo-oxazolidin-5-ylmethyl)-acetamide

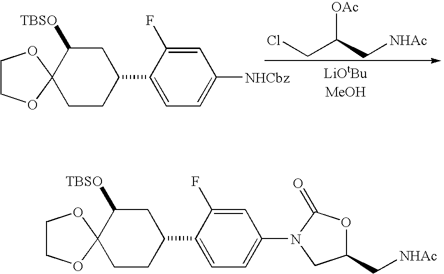

To a solution of {4-[6-(tert-butyl-dimethyl-silanyloxy)-1,4-dioxa-spiro[4.5]dec-8-yl]-3-fluoro-phenyl}-carbamic acid benzyl ester (202 mg, 0.39 mmol) in DMF (0.3 mL) and methanol (32 μL, 2.02 eq) was added a solution of lithium t-butoxide in THF (1.0 M solution, 1.2 mL, 1.2 mmol) at 0° C. Then (1S)-2-(acetylamino)-1-(chloromethyl)ethyl acetate (152 mg, 0.78 mmol) was added and the resulting reaction mixture was warmed up to room temperature and stirred for 17 hours. Saturated aqueous ammonium chloride (2 mL) was then added followed by water (10 mL). The solution was extracted with dichloromethane, washed with brine, and dried over MgSO₄. Solvent was removed and the residue was purified by prep TLC (10% methanol/dichloromethane) to give the desired product (110 mg, 54%) as a viscous liquid. HPLC: retention time 7.15 minutes, Purity >99%; [M+Na]⁺ 545.3.
N-{3-[3-Fluoro-4-(6-hydroxy-1,4-dioxa-spiro[4.5]dec-8-yl)-phenyl]-2-oxo-oxazolidin-5-ylmethyl}-acetamide

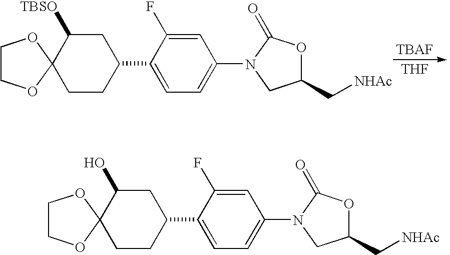

To a solution of N-(3-{4-[6-(tert-butyl-dimethyl-silanyloxy)-1,4-dioxa-spiro[4.5]dec-8-yl]-3-fluoro-phenyl}-2-oxo-oxazolidin-5-ylmethyl)-acetamide (112 mg, 0.21 mmol) in 2 mL of THF was added 0.64 mL of tetrabutylammonium fluoride (TBAF, 1.0 M in THF, 0.64 mmol). The reaction mixture was then stirred overnight at room temperature. After the reaction was completed, the mixture was taken up in dichloromethane, washed with brine and dried over MgSO₄. Solvent was removed and the residue was purified by prep TLC (20% methanol/EtOAc). The product was obtained as a white solid (44 mg, 50%). HPLC: retention time 4.55 minutes, purity 99%; [M+Na]⁺ 431.3.
N-{3-[3-Fluoro-4-(3-hydroxy-4-oxo-cyclohexyl)-phenyl]-2-oxo-oxazolidin-5-ylmethyl}-acetamide

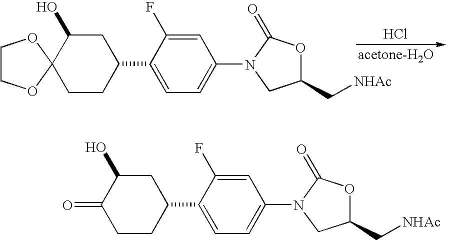

To a stirred solution of N-{3-[3-fluoro-4-(6-hydroxy-1,4-dioxa-spiro[4.5]dec-8-yl)-phenyl]-2-oxo-oxazolidin-5-ylmethyl}-acetamide (40 mg, 0.098 mmol) in 4 mL of acetone and 0.5 mL of water was added 3 drops of HCl (4.0 M solution in dioxane). The reaction mixture was then refluxed overnight, cooled to room temperature and quenched with diluted NaHCO₃ solution. The aqueous solution was extracted with DCM, washed with brine and dried over MgSO₄. Solvent was removed and the residue was purified by preparative TLC (20% methanol/EtOAc). The product was obtained as a white solid (44 mg, 56%). ¹H NMR (300 MHz, DMSO) δ 8.24 (t, J=5.4 Hz, 1H), 7.46 (dd, J=13.2 Hz, 2.1 Hz, 1H), 7.40-7.21 (m, 2H), 5.06 (d, J=4.5 Hz, 1H), 4.70 (m, 1H), 4.31 (m, 1H), 4.09 (t, J=9.0 Hz, 1H), 3.71 (dd, J=9.0 Hz, 6.3 Hz, 1H), 2.66 (td, J=13.8 Hz, 5.7 Hz, 1H), 2.31-1.72 (m, 7H); HPLC: retention time 4.27 minutes, purity >95%; [M+H]⁺ 365.4.

### Example 26

### Preparation of N-{3-[3-Fluoro-4-(3-fluoro-4-hydroxy-cyclohexyl)-phenyl]-2-oxo-oxazolidin-5-ylmethyl}-acetamide

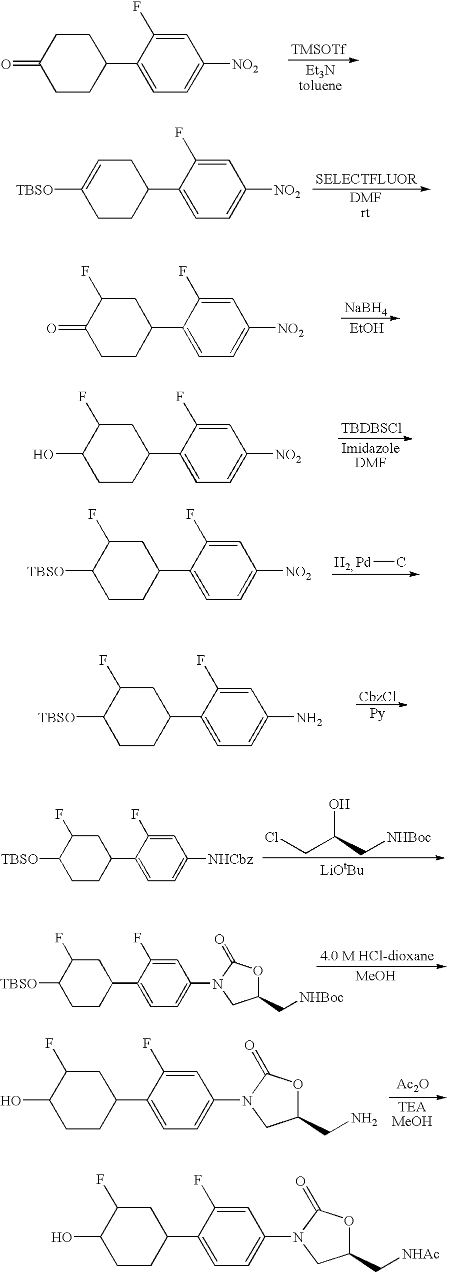 2-Fluoro-4-(2-fluoro-4-nitro-phenyl)-cyclohexanone

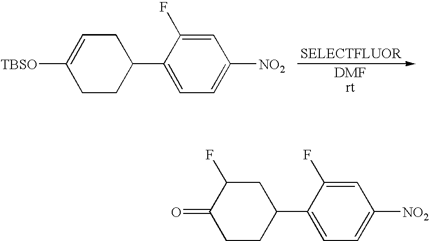

tert-Butyl-[4-(2-fluoro-4-nitro-phenyl)-cyclohex-1-enyloxy]-dimethyl-silane (130 mg, 0.42 mmol) was dissolved in 2 mL of DMF under nitrogen. Selectfluor (149 mg, 0.42 mmol) was added and the reaction mixture was stirred overnight at room temperature. The reaction was quenched with water and extracted with dichloromethane. The organic layer was washed with brine and dried over MgSO₄. Solvent was removed and the residue was purified by preparative TLC (50% EtOAc/hexanes). The product was obtained as a solid (80 mg, 75%). HPLC: retention time 5.05 minutes, purity >95%.
2-Fluoro-4-(2-fluoro-4-nitro-phenyl)-cyclohexanol

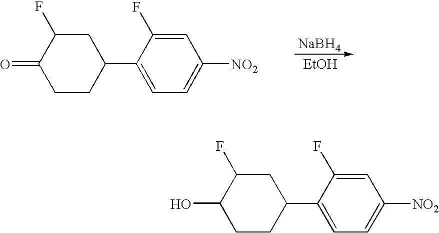

2-Fluoro-4-(2-fluoro-4-nitro-phenyl)-cyclohexanone (538 mg, 2.11 mmol) was dissolved in 8 mL of ethanol, and then 160 mg (4.22 mmol) of sodium borohydride was added. After one hour, the reaction was quenched slowly with 10% HCl and most of the solvent was removed under vacuum. The residue was poured into water and extracted with ethyl acetate. The organic layer was washed with brine and dried over MgSO₄. Solvent was removed and the residue was purified by preparative TLC (50% EtOAc/hexanes) to give two major products:
upper spot (diastereomer-1, 146 mg): HPLC: retention time 5.40 minutes, purity >95%.
lower spot (diastereomer-2, 153 mg): HPLC: retention time 5.39 minutes, purity >95%.
   tert-Butyl-[2-fluoro-4-(2-fluoro-4-nitro-phenyl)-cyclohexyloxy]-dimethyl-silane

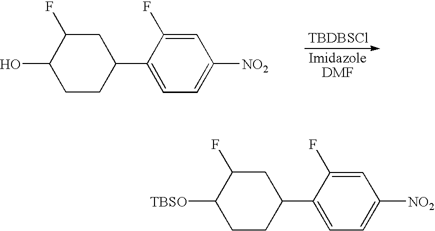

To a solution of 2-fluoro-4-(2-fluoro-4-nitro-phenyl)-cyclohexanol (diastereomer-1, 203 mg, 0.79 mmol) in 3 mL of DMF was added imidazole (161 mg, 2.37 mmol), followed by tert-butyldimethylsilyl chloride (TBS, 178 mg, 1.18 mmol) at 0° C. The reaction mixture was then warmed up to room temperature and stirred for another hour. The reaction was quenched with water, extracted with ethyl acetate, and the organic layer was washed with brine and dried over MgSO₄. Solvent was removed to give the desired product as a viscous liquid (270 mg, 92%). HPLC: retention time 8.25 minutes, purity >95%.

Diastereomer-2 (250 mg, 0.67 mmol) was carried out in a similar manner and the product was obtained as a liquid (219 mg, 96%). HPLC: retention time 8.20 minutes, purity >95%.
4-[4-(tert-Butyl-dimethyl-silanyloxy)-3-fluoro-cyclohexyl]-3-fluoro-phenylamine

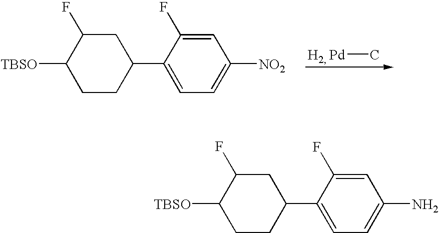

tert-Butyl-[2-fluoro-4-(2-fluoro-4-nitro-phenyl)-cyclohexyloxy]-dimethyl-silane (diastereomer-1, 260 mg, 0.70 mmol) was dissolved in 12 mL of methanol, and then 30 mg of Pd—C (10% on activated carbon) was added. The flask was then connected to vacuum and purged with hydrogen. The reaction mixture was stirred under hydrogen overnight, filtered through a short celite pad, and washed with methanol. Solvent was then removed to give the desired product as a liquid (220 mg, 92%). HPLC: retention time 6.65 minutes, purity >95%; [M+H]⁺ 342.5.

Diastereomer-2 (193 mg, 0.75 mmol) was carried out in a similar manner and the product was obtained as a liquid (250 mg, 90%). HPLC: retention time 6.38 minutes, purity >95%; [M+H]⁺ 342.5.
{4-[4-(tert-Butyl-dimethyl-silanyloxy)-3-fluoro-cyclohexyl]-3-fluoro-phenyl}-carbamic acid benzyl ester

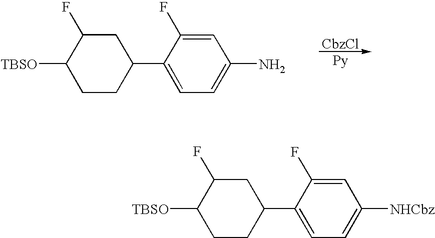

Benzyl chloroformate (106 μL, 0.74 mmol) was added dropwise to a mixture of the 4-[4-(tert-butyl-dimethyl-silanyloxy)-3-fluoro-cyclohexyl]-3-fluoro-phenylamine (diastereomer-1, 210 mg, 0.62 mmol) and pyridine (120 μL, 1.48 mmol) in DCM (5 mL) at 0° C. The reaction mixture was warmed up to room temperature and stirred for 1 hour, then it was poured into water and extracted with ethyl acetate (20 mL×2). The organic layer was collected and washed with brine, and dried over MgSO₄. Solvent was removed and the residue was purified by prep TLC (20% EtOAc/hexanes) to give the desired product as a white solid (220 mg, 75%). HPLC: retention time 8.50 minutes, purity >95%; [M+H]⁺ 476.5.

Diastereomer-2 (210 mg, 0.62 mmol) was carried out in a similar manner and the product was obtained as a solid (270 mg, 92%). HPLC: retention time 8.25 minutes, purity >95%; [M+H]⁺ 476.5.
N-{3-[3-Fluoro-4-(3-fluoro-4-hydroxy-cyclohexyl)-phenyl]-2-oxo-oxazolidin-5-ylmethyl}-acetamide

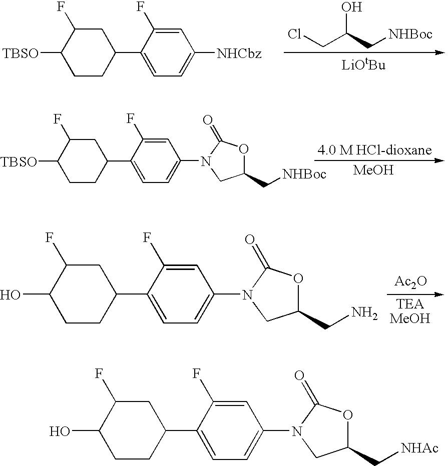

To a solution of {4-[4-(tert-butyl-dimethyl-silanyloxy)-3-fluoro-cyclohexyl]-3-fluoro-phenyl}-carbamic acid benzyl ester (diastereomer-1, 53 mg, 0.11 mmol) and acetic acid 1(S)-(tert-butoxycarbonylamino-methyl)-2-chloro-ethyl ester (29 mg, 0.14 mmol) in DMF was added a solution of lithium t-butoxide (1.0 M solution in THF, 0.27 mL, 0.27 mmol) at 0° C. The reaction mixture was then warmed up to room temperature and stirred overnight. Saturated aqueous ammonium chloride (2 mL) was added followed by water (20 mL). The reaction mixture was extracted with dichloromethane, washed with brine, and the organic layer was dried over MgSO₄. Solvent was removed and the residue was purified by preparative TLC (50% EtOAc/hexanes) to give the product as an oil.

To a solution of (3-{4-[4-(tert-butyl-dimethyl-silanyloxy)-3-fluoro-cyclohexyl]-3-fluoro-phenyl}-2-oxo-oxazolidin-5-ylmethyl)-carbamic acid tert-butyl ester in 1 mL of methanol was added 0.5 mL of HCl solution (4.0 M in dioxane). The reaction mixture was then stirred for two hours at room temperature. Solvent was removed under vacuum and the residue was dried under high vacuum.

The free amine was dissolved in 1 mL of methanol and 61 μL (0.44 mmol) of triethylamine followed by 21 μL (0.22 mmol) of acetic were added. The reaction was stirred for one hour at room temperature, condensed, and purified by preparative TLC (10% methanol/dichloromethane) to give the product as a white solid (30 mg, 73% for three steps). ¹H NMR (300 MHz, DMSO) δ 8.23 (t, J=5.7 Hz, 1H), 7.46-7.21 (m, 3H), 5.15 (d, J=4.5 Hz, 1H), 4.70 (m, 1H), 4.47-4.22 (m, 1H), 4.09 (t, J=9.3 Hz, 1H), 3.70 (dd, J=9.3 Hz, 6.6 Hz, 1H), 2.89 (t, J=12.0 Hz, 1H), 2.02-1.30 (m, 9H); HPLC: R.T. 4.50 minutes, purity >95%; [M+H]⁺ 369.5.

Diastereomer-2 (53 mg, 0.11 mmol) was carried out in a similar manner and the product was obtained as a solid (32 mg, 78% for three steps). ¹H NMR (300 MHz, DMSO) δ 8.23 (t, J=5.7 Hz, 1H), 7.45-7.21 (m, 3H), 4.93 (d, J=5.7 Hz, 1H), 4.70 (m, 1H), 4.09 (t, J=9.0 Hz, 1H), 3.70 (dd, J=9.3 Hz, 6.3 Hz, 1H), 3.03 (t, J=12.0 Hz, 1H), 1.98-1.55 (m, 9H); HPLC: R.T. 4.48 minutes, purity >95%; [M+H]⁺ 369.5.

### Example 27

### N-{3-[3-Fluoro-4-(3-fluoro-4-hydroxy-cyclohexyl)-phenyl]-2-oxo-oxazolidin-5-ylmethyl}-propionamide

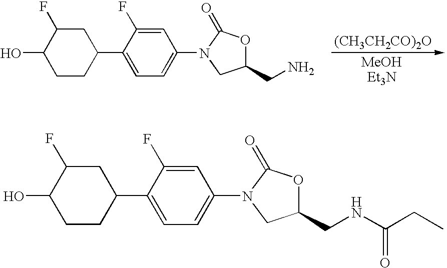

5-Aminomethyl-3-[3-fluoro-4-(3-fluoro-4-hydroxy-cyclohexyl)-phenyl]-oxazolidin-2-one (diastereomer-2, Example 26 119 mg, 0.36 mmol) was dissolved in 2 mL of methanol and 203 μL of triethylamine (1.44 mmol) followed by 93 μL of propionic anhydide (0.72 mmol) were added. The reaction was stirred for one hour at room temperature, condensed, and purified by preparative TLC (10% methanol/dichloromethane) to give the product as a white solid (60 mg, 43%). ¹H NMR (300 MHz, DMSO) δ 8.15 (t, J=6.0 Hz, 1H), 7.44-7.20 (m, 3H), 4.93 (d, J=5.4 Hz, 1H), 4.70 (m, 1H), 4.08 (t, J=9.0 Hz, 1H), 3.71 (dd, J=9.3 Hz, 6.3 Hz, 1H), 3.03 (t, J=12.0 Hz, 1H), 2.11-1.55 (m, 7H), 0.94 (t, J=7.5 Hz, 3H); HPLC: retention time 3.95 minutes, purity >95%; [M+H]⁺ 383.3.

### Example 28

### Preparation of 2,2-Difluoro-N-{3-[3-fluoro-4-(3-fluoro-4-hydroxy-cyclohexyl)-phenyl]-2-oxo-oxazolidin-5-ylmethyl}-thioacetamide

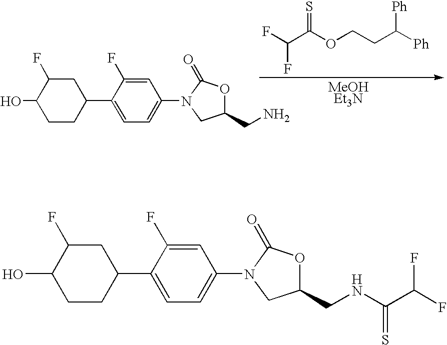

To a solution of 5-aminomethyl-3-[3-fluoro-4-(3-fluoro-4-hydroxy-cyclohexyl)-phenyl]-oxazolidin-2-one (95 mg, 0.29 mmol) in 2 mL of MeOH-dichloromethane (1:9) was added difluoro-thioacetic acid O-(3,3-diphenyl-propyl) ester (107 mg, 0.35 mmol) followed by triethylamine (81 μL, 0.58 mmol). The reaction was stirred overnight at room temperature, condensed, and purified by preparative TLC (85% EtOAc/hexanes) to give the product as a white solid (50 mg, 41%). ¹H NMR (300 MHz, DMSO) δ 7.46-7.23 (m, 3H), 6.48 (t, J=55.2 Hz, 1H), 5.15 (d, J=4.8 Hz, 1H), 5.00 (m, 1H), 4.16 (t, J=9.3 Hz, 1H), 3.83 (dd, J=9.0 Hz, 6.0 Hz, 1H), 2.90 (t, J=12.0 Hz, 1H), 1.75-1.34 (m, 2H); HPLC: retention time 5.49 minutes, purity >95%; [M+H]⁺ 421.4.

Diastereomer-2 (34 mg, 0.11 mmol) was carried out in a similar manner and the product was obtained as a solid (20 mg, 46%). ¹H NMR (300 MHz, DMSO) δ 7.45-7.22 (m, 3H), 6.48 (t, J=55.2 Hz, 1H), 5.05-4.69 (m, 2H), 4.16 (t, J=9.3 Hz, 1H), 3.83 (dd, J=9.6 Hz, 6.3 Hz, 1H), 3.04 (t, J=12.0 Hz, 1H), 1.98-1.55 (m, 3H); HPLC: retention time 5.48 minutes, purity >95%; [M+H]⁺ 421.4.

The following illustrates representative pharmaceutical dosage forms, containing a compound of Formula I (“Invention Compound”), for therapeutic or prophylactic use in humans.

| (i) | Tablet | mg/tablet |
|---|---|---|
| | ‘Invention Compound’ | 10-1000 |
| | Lactose | 50.0 |
| | Corn Starch (for mix) | 10.0 |
| | Corn Starch (paste) | 10.0 |
| | Magnesium Stearate (1%) | 3.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) | Tablet | mg/capsule |
|---|---|---|
| | ‘Invention Compound | 10-1000 |
| | Colloidal Silicon Dioxide | 1.5 |
| | Lactose | 465.5 |
| | Pregelatinized Starch | 120.0 |
| | Magnesium Stearate (1%) | 3.0 |

| | Preparation for | |
|---|---|---|
| (iii) | Oral Solution | Amount |
| | ‘Invention Compound’ | 10-1000 |
| | Sorbitol Solution (70% N.F.) | 40 mL |
| | Sodium Benzoate | 20 mg |
| | Saccharin | 5 mg |
| | Cherry Flavor | 20 mg |
| | Distilled Water q.s. | 100 mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| (v) | Injection 1 (1 mg/mL) | Amount |
|---|---|---|
| | ‘Invention Compound’ | 10-1000 |
| | Dibasic Sodium Phosphate | 12.0 |
| | Monobasic Sodium Phosphate | 0.7 |
| | Sodium Chloride | 4.5 |
| | 1.0 N Sodium hydroxide solution | q.s. |
| | (pH adjustment to 7.0-7.5) | |
| | Water for injection | q.s. ad 1 mL |

| (vi) | Injection 2 (10 mg/mL) | Amount |
|---|---|---|
| | ‘Invention Compound’ | 10-1000 |
| | Dibasic Sodium Phosphate | 1.1 |
| | Monobasic Sodium Phosphate | 0.3 |
| | Polyethylene glyco 400 | 200.0 |
| | 0.1 N hydrochloric acid solution | q.s. |
| | (pH adjustment to 7.0-7.5) | |
| | Water for injection | q.s. ad 1 mL |

| (vii) | Injection 2 (10 mg/mL) | Amount |
|---|---|---|
| | ‘Invention Compound’ | 10-1000 |
| | Oleic Acid | 10.0 |
| | Trichloromonofluoromethane | 5,000.0 |
| | Dichlorodifluoromethane | 10,000.0 |
| | Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I 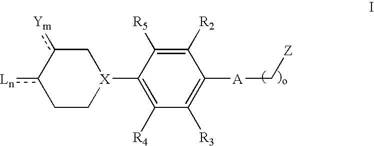
or a pharmaceutically acceptable salt thereof wherein:
“- - - ” is a bond or is absent;
A is a structure selected from the group consisting of i, ii, iii, and iv 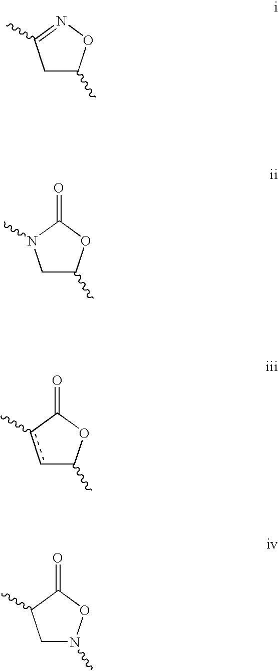
wherein “- - - ” is a bond or is absent and “ [symbol] ” indicate points of attachment;
X is N, or C;
Z is
(h) NHC(═O)R¹,
(i) NHC(═S)R¹,
(j) NH-het¹,
(k) O-het¹,
(l) S-het¹,
(m) het²; or
(n) CONHR¹, wherein
R¹ is
(m) H,
(n) NH₂,
(o) NHC₁₋₄alkyl,
(p) C₁₋₄alkyl,
(q) C₂₋₄alkenyl,
(r) C₁₋₄heteroalkyl,
(s) (CH₂)ₚC(═O)C₁₋₄alkyl,
(t) OC₁₋₄alkyl, except when o=0;
(u) SC₁₋₄alkyl, except when o=0;
(v) (CH₂)ₚC₃₋₆cycloalkyl,
(w) CH₂C(═O)-aryl, or
(x) CH₂C(═O)-het¹;
R², R³, R⁴ and R⁵ are each independently
(g) H,
(h) Cl,
(i) F,
(j) CH₃,
(k) NH₂, or
(l) OH;
L and Y are each independently
(g) H,
(h) OH,
(i) F,
(j) O,
(k) NOH,
(l) NOR;
m, n, o, p are each independently 0 or 1.

2. The compound of claim 1, wherein R¹ is C₁₋₄alkyl, optionally substituted with one, two or three fluoro (F), or chloro (Cl).

3. The compound of claim 2, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

4. The compound of claim 2, wherein R² and R³ are each independently H or F.

5. The compound of claim 2, wherein at least one of R² and R³ is F.

6. The compound of claim 2, wherein R² and R³ are F.

7. The compound of claim 2, wherein X is C or N.

8. The compound of claim 2, wherein L is O or F.

9. The compound of claim 2, wherein n is 1.

10. The compound of claim 2, wherein Y is O or F.

11. The compound of claim 2, wherein m is 1.

12. The compound of claim 2, wherein W is O or F.

13. The compound of claim 2, wherein het¹ is isoxazolyl, isothiazolyl, 1,2,5-thiadiazolyl, or pyridyl.

14. The compound of claim 2, wherein het² is 1,2,3-triazolyl.

15. The compound of claim 2, wherein the A structure i, ii, or iii has an optical configuration as depicted below:

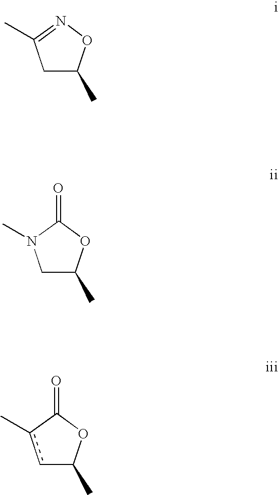

16. The compound of claim 2, wherein 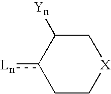
is selected from the group consisting of

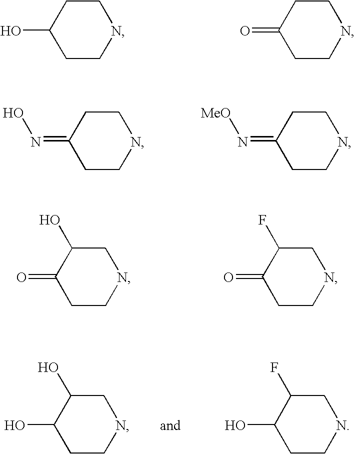

17. The compound of claim 2, wherein 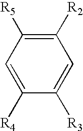
is selected from the group consisting of

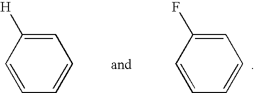

18. The compound of claim 2, wherein 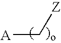
is selected from the group consisting of

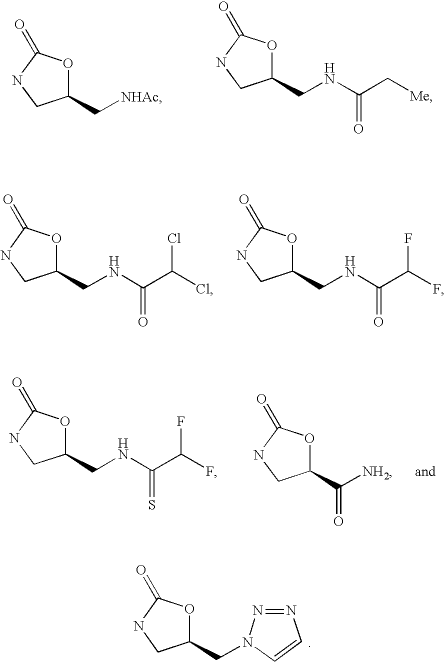

19. A compound or a pharmaceutically acceptable salt thereof which is
(a) N-{3-[4-(3,4-Dihydroxy-cyclohexyl)-phenyl]-2-oxo-oxazolidin-5-ylmethyl}-acetamide;
(b) 2,2-Dichloro-N-{3-[4-(3,4-dihydroxy-cyclohexyl)-phenyl]-2-oxo-oxazolidin-5-ylmethyl}-acetamide;
(c) N-{3-[4-(3,4-Dihydroxy-cyclohexyl)-phenyl]-2-oxo-oxazolidin-5-ylmethyl}-2,2-difluoro-acetamide;
(d) N-{3-[4-(3,4-Dihydroxy-cyclohexyl)-phenyl]-2-oxo-oxazolidin-5-ylmethyl}-2,2-difluoro-thioacetamide;
(e) N-{3-[4-(3,4-Dihydroxy-cyclohexyl)-3-fluoro-phenyl]-2-oxo-oxazolidin-5-ylmethyl}-acetamide;
(f) 2,2-Dichloro-N-{3-[4-(3,4-dihydroxy-cyclohexyl)-3-fluoro-phenyl]-2-oxo-oxazolidin-5-ylmethyl}-acetamide;
(g) N-{3-[4-(3,4-Dihydroxy-cyclohexyl)-3-fluoro-phenyl]-2-oxo-oxazolidin-5-yl methyl}-2,2-difluoro-acetamide;
(h) N-{3-[4-(3,4-Dihydroxy-cyclohexyl)-3-fluoro-phenyl]-2-oxo-oxazolidin-5-ylmethyl}-2,2-difluoro-thioacetamide;
(i) N-{3-[3-Fluoro-4-(4-hydroxy-cyclohexyl)-phenyl]-2-oxo-oxazolidin-5-ylmethyl}-acetamide;
(j) N-{3-[3-Fluoro-4-(4-oxo-cyclohexyl)-phenyl]-2-oxo-oxazolidin-5-ylmethyl}-acetamide;
(k) 2,2-Difluoro-N-{3-[3-fluoro-4-(4-hydroxy-cyclohexyl)-phenyl]-2-oxo-oxazolidin-5-ylmethyl}-thioacetamide;
(l) N-{3-[3-Fluoro-4-(4-hydroxyimino-cyclohexyl)-phenyl]-2-oxo-oxazolidin-5-ylmethyl}-acetamide;
(m) N-{3-[3-Fluoro-4-(4-hydroxy-cyclohexyl)-phenyl]-2-oxo-oxazolidin-5-ylmethyl}-propionamide;
(n) N-{3-[3-Fluoro-4-(3-hydroxy-4-oxo-cyclohexyl)-phenyl]-2-oxo-oxazolidin-5-ylmethyl}-acetamide;
(o) 1-[2-Fluoro-4-(2-oxo-5-[1,2,3]triazol-1-ylmethyl-oxazolidin-3-yl)-phenyl]-3-hydroxy-piperidin-4-one;
(u) 3-[4-(3,4-Dihydroxy-piperidin-1-yl)-3-fluoro-phenyl]-5-[1,2,3]triazol-1-ylmethyl-oxazolidin-2-one;
(v) 3-[3-Fluoro-4-(4-hydroxy-cyclohexyl)-phenyl]-2-oxo-oxazolidine-5-carboxylic acid amide;
(w) 3-[3-Fluoro-4-(4-oxo-cyclohexyl)-phenyl]-2-oxo-oxazolidine-5-carboxylic acid amide;
(x) N-{3-[3-Fluoro-4-(3-fluoro-4-hydroxy-cyclohexyl)-phenyl]-2-oxo-oxazolidin-5-ylmethyl}-acetamide; or
2,2-Difluoro-N-{3-[3-fluoro-4-(3-fluoro-4-hydroxy-cyclohexyl)-phenyl]-2-oxo-oxazolidin-5-ylmethyl}-thioacetamide.

20. A compound of formula II 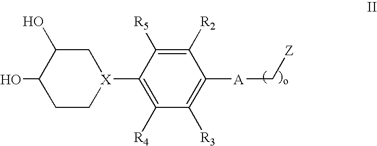
or a pharmaceutically acceptable salt thereof, wherein A, X, Z, R₂, R₃, R⁴, R₅, and o have the definitions as provided in claim 1.

21. The compound of claim 20, wherein R¹ is C₁₋₄alkyl, optionally substituted with one, two or three fluoro (F), or chloro (Cl).

22. The compound of claim 21, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

23. The compound of claim 21, wherein R² and R³ are each independently H or F.

24. The compound of claim 21, wherein at least one of R² and R³ is F.

25. The compound of claim 21, wherein R² and R³ are F.

26. The compound of claim 21, wherein X is C or N.

27. The compound of claim 21, wherein Y is O or F.

28. The compound of claim 21, wherein het¹ is isoxazolyl, isothiazolyl, 1,2,5-thiadiazolyl, or pyridyl.

29. The compound of claim 21, wherein het² is 1,2,3-triazolyl.

30. The compound of claim 21, wherein the A structure i, ii, or iii has an optical configuration as depicted below:

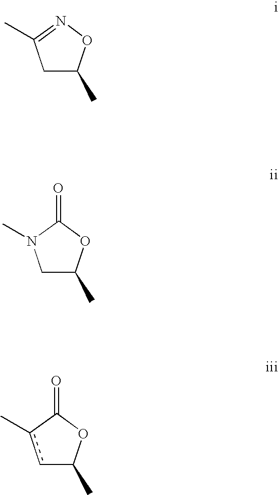

31. The compound of claim 20, wherein

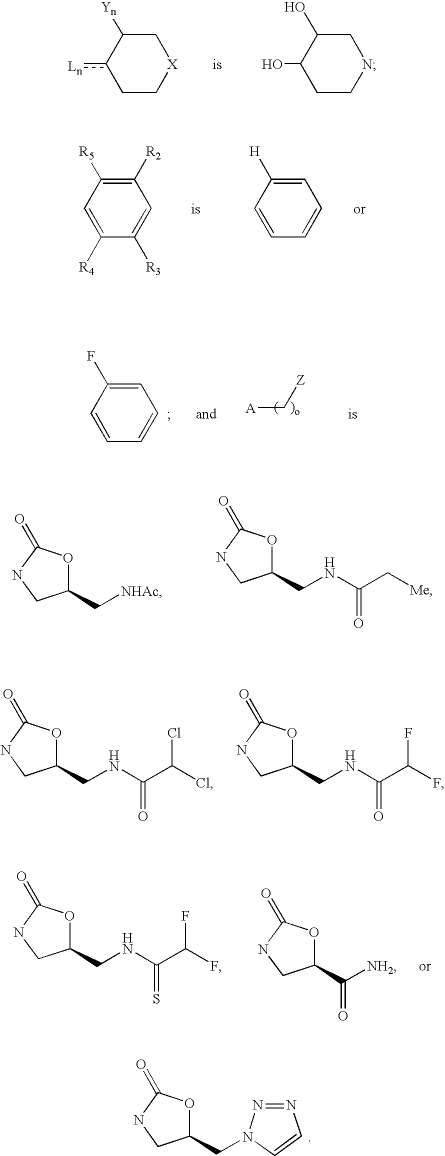

32. The compound of formula III 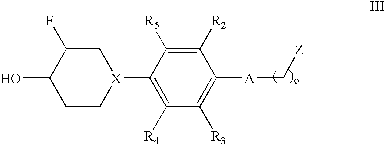
or a pharmaceutically acceptable salt thereof, wherein A, X, Z, R₂, R₃, R⁴, R₅, and 0 have the definitions as provided in claim 1.

33. The compound of claim 32, wherein R¹ is C₁₋₄alkyl, optionally substituted with one, two or three fluoro (F), or chloro (CI).

34. The compound of claim 32, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, CH₂C(═O)aryl, CF₃, cyclopropyl.

35. The compound of claim 32, wherein R² and R³ are each independently H or F.

36. The compound of claim 32, wherein at least one of R² and R³ is F.

37. The compound of claim 32, wherein R² and R³ are F.

38. The compound of claim 32, wherein X is C or N.

39. The compound of claim 32, wherein Y is O or F.

40. The compound of claim 32, wherein het¹ is isoxazolyl, isothiazolyl, 1,2,5-thiadiazolyl, or pyridyl.

41. The compound of claim 32, wherein het² is 1,2,3-triazolyl.

42. The compound of claim 32, wherein the A structure i, ii, or iii has an optical configuration as depicted below:

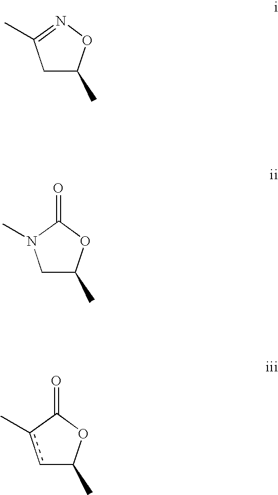

43. The compound of claim 32, wherein

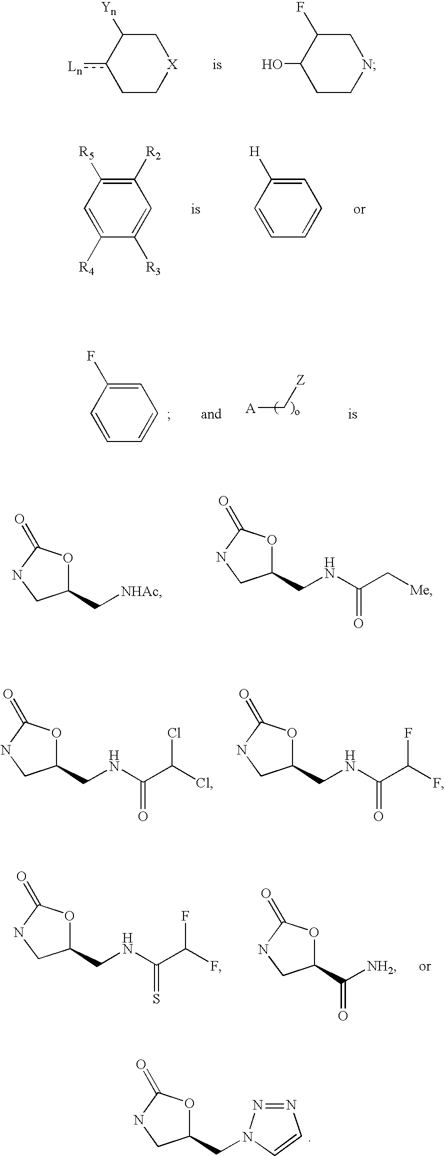

44. A compound of formula IV 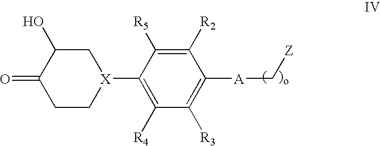
or a pharmaceutically acceptable salt thereof, wherein A, X, Z, R₂, R₃, R⁴, R₅, and o have the definitions as provided in claim 1.

45. The compound of claim 44, wherein R¹ is C₁₋₄alkyl, optionally substituted with one, two or three fluoro (F), or chloro (Cl).

46. The compound of claim 44, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

47. The compound of claim 44, wherein R² and R³ are each independently H or F.

48. The compound of claim 44, wherein at least one of R² and R³ is F.

49. The compound of claim 44, wherein R² and R³ are F.

50. The compound of claim 44, wherein X is C or N.

51. The compound of claim 44, wherein Y is O or F.

52. The compound of claim 44, wherein het¹ is isoxazolyl, isothiazolyl, 1,2,5-thiadiazolyl, or pyridyl.

53. The compound of claim 44, wherein het² is 1,2,3-triazolyl.

54. The compound of claim 44, wherein the A structure i, ii, or iii has an optical configuration as depicted below:

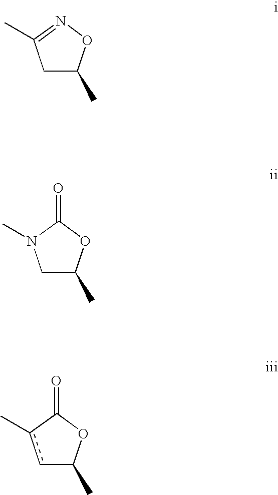

55. The compound of claim 44, wherein

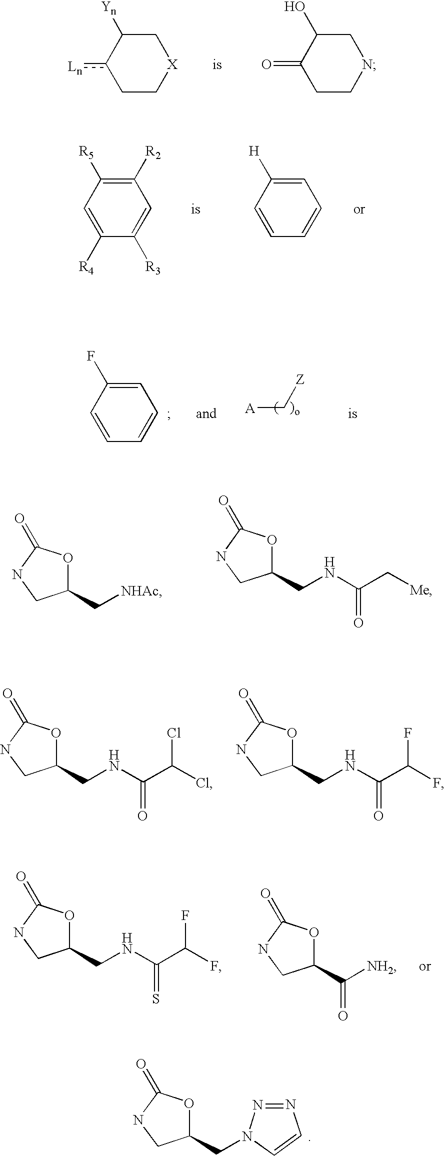

56. A compound of formula V 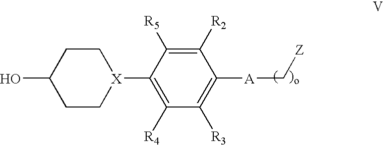
or a pharmaceutically acceptable salt thereof, wherein A, X, Z, R₂, R₃, R⁴, R₅, and o have the definitions as provided in claim 1.

57. The compound of claim 56, wherein R¹ is C₁₋₄alkyl, optionally substituted with one, two or three fluoro (F), or chloro (Cl).

58. The compound of claim 56, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

59. The compound of claim 56, wherein R² and R³ are each independently H or F.

60. The compound of claim 56, wherein at least one of R² and R³ is F.

61. The compound of claim 56, wherein R² and R³ are F.

62. The compound of claim 56, wherein X is C or N.

63. The compound of claim 56, wherein Y is O or F.

64. The compound of claim 56, wherein het¹ is isoxazolyl, isothiazolyl, 1,2,5-thiadiazolyl, or pyridyl.

65. The compound of claim 56, wherein het² is 1,2,3-triazolyl.

66. The compound of claim 56, wherein the A structure i, ii, or iii has an optical configuration as depicted below:

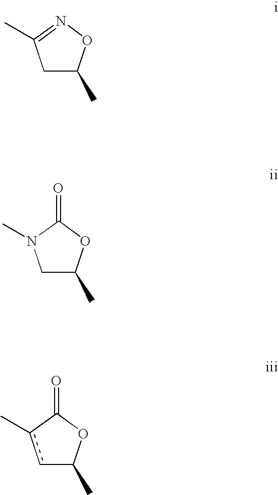

67. The compound of claim 56, wherein

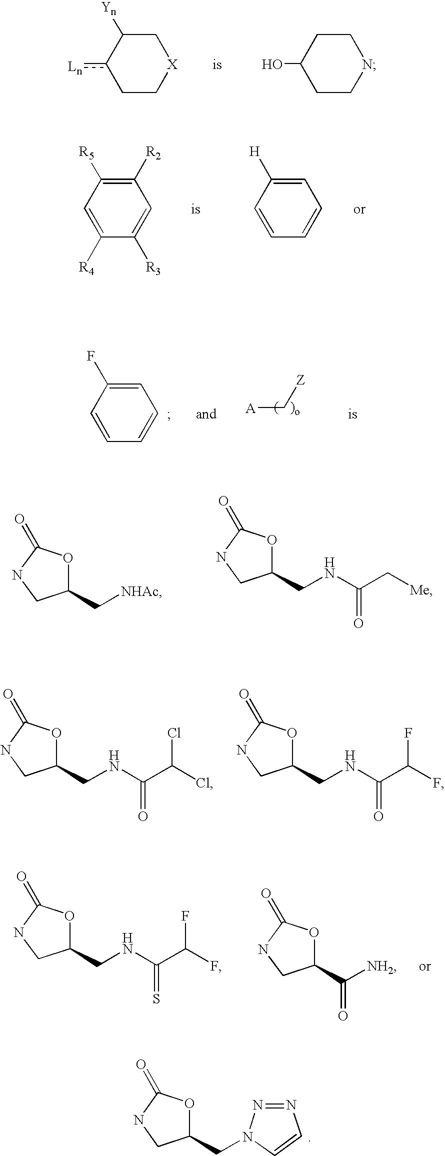

68. A compound of formula VI 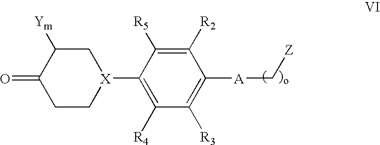
or a pharmaceutically acceptable salt thereof, wherein A, X, Z, R₂, R₃, R⁴, R₅, and o have the definitions as provided in claim 1.

69. The compound of claim 68, wherein R¹ is C₁₋₄alkyl, optionally substituted with one, two or three fluoro (F), or chloro (Cl).

70. The compound of claim 68, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

71. The compound of claim 68, wherein R and R³ are each independently H or F.

72. The compound of claim 68, wherein at least one of R² and R³ is F.

73. The compound of claim 68, wherein R and R³ are F.

74. The compound of claim 68, wherein X is C or N.

75. The compound of claim 68, wherein Y is O or F.

76. The compound of claim 68, wherein het¹ is isoxazolyl, isothiazolyl, 1,2,5-thiadiazolyl, or pyridyl.

77. The compound of claim 68, wherein het² is 1,2,3-triazolyl.

78. The compound of claim 68, wherein the A structure i, ii, or iii has an optical configuration as depicted below:

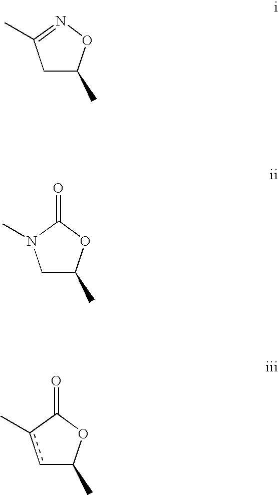

79. The compound of claim 68, whererin

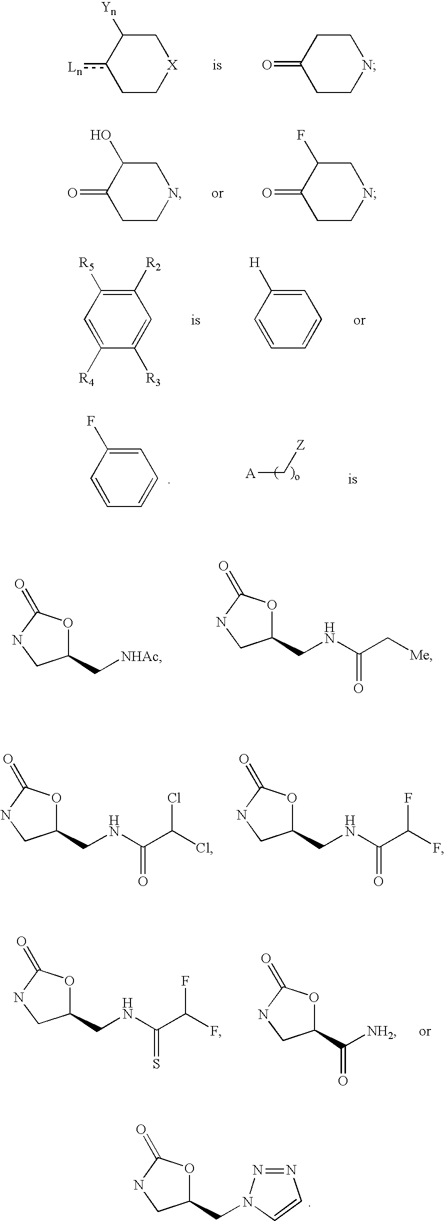

80. A compound of formula VII 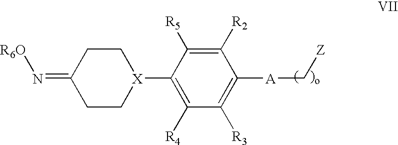
or a pharmaceutically acceptable salt thereof, wherein A, X, Z, R₂, R₃, R⁴, R₅, and o have the definitions as provided in claim 1 and R₆ is H or (C₁-C₆)alkyl.

81. The compound of claim 80, wherein R¹ is C₁₋₄alkyl, optionally substituted with one, two or three fluoro (F), or chloro (Cl).

82. The compound of claim 80, wherein R¹ is CH₃, CH₂CH₃, CHF₂, or CHCl₂, CH₂CF₃, CF₂CH₃, H, —CH═CH-aryl, —CH₂C(═O)C₁₋₄alkyl, —CH₂C(═O)aryl, CF₃, cyclopropyl.

83. The compound of claim 80, wherein R² and R³ are each independently H or F.

84. The compound of claim 80, wherein at least one of R² and R³ is F.

85. The compound of claim 80, wherein R² and R³ are F.

86. The compound of claim 80, wherein X is C or N.

87. The compound of claim 80, wherein Y is O or F.

88. The compound of claim 80, wherein het¹ is isoxazolyl, isothiazolyl, 1,2,5-thiadiazolyl, or pyridyl.

89. The compound of claim 80, wherein het² is 1,2,3-triazolyl.

90. The compound of claim 80, wherein the A structure i, ii, or iii has an optical configuration as depicted below:

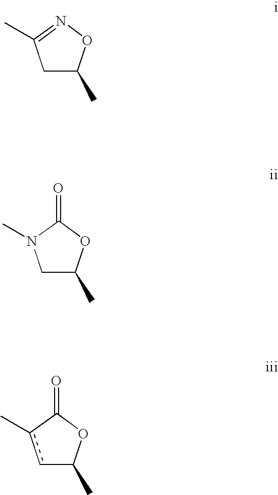

91. The compound of claim 80, wherein

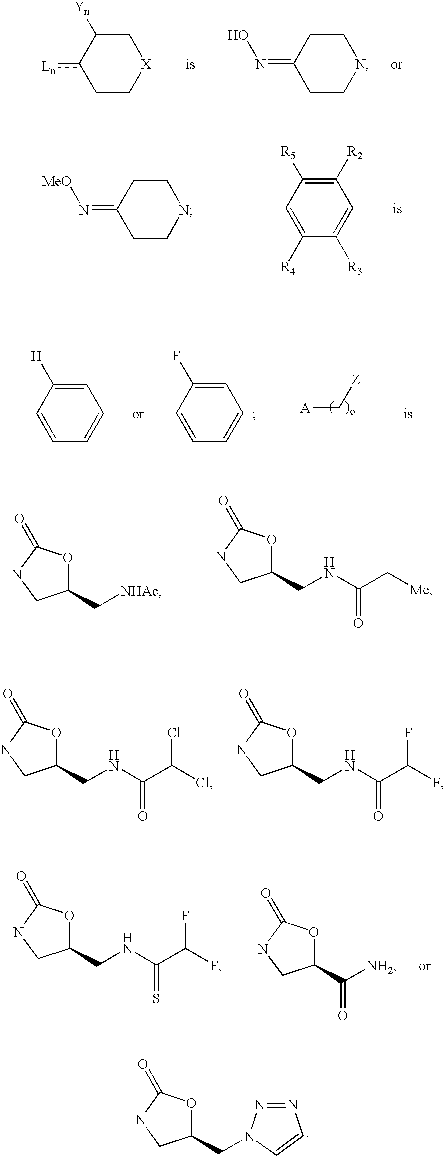

92. The invention is further directed to a pharmaceutical composition comprising a compound of formula I, II, III, IV, V, VI, or VII or a pharmaceutically acceptable salt thereof, admixed with a pharmaceutically acceptable excipent, carrier, or diluent.

93. The invention is further directed to a method for treating a microbial infection in a mammal in need of such treatment, comprising administering a therapeutically effective amount of a compound of formula I, II, III, IV, V, I, or VII or a pharmaceutically acceptable salt thereof.

94. invention is further directed to a method for treating gram-positive microbial infections in a mammal in need of such treatment, comprising administering a therapeutically effective amount of a compound of formula I, II, III, IV, V, VI, or VII or a pharmaceutically acceptable salt thereof.

95. The invention is further directed to a method for treating a gram-negative microbial infection in a mammal in need of such treatment, comprising administering a therapeutically effective amount of a compound of formula I, II, III, IV, V, VI, or VII or a pharmaceutically acceptable salt thereof.
